(19) United States
(12) Patent Application Publication
(10) Pub. No.: US 2026/0308507 A1
(43) Pub. Date: Oct. 8, 2026

(54) APPARATUS FOR QUANTITATIVE STUDY OF CELLULAR INTERACTIONS WITH MECHANICAL WAVES

(71) Applicant: ARIZONA BOARD OF REGENTS ON BEHALF OF THE UNIVERSITY OF ARIZONA, Tucson, AZ (US)

(72) Inventors: Jian GU, Phoenix, AZ (US); Frederic ZENHAUSERN, Phoenix, AZ (US); Adam AKKAD, Phoenix, AZ (US); Nabhan FAKRUDIN, Phoenix, AZ (US)

(21) Appl. No.: 19/631,845

(22) Filed: Mar. 27, 2026

Related U.S. Application Data
(63) Continuation-in-part of application No. PCT/US2024/049098, filed on Sep. 27, 2024.
(60) Provisional application No. 63/541,663, filed on Sep. 29, 2023.

(51) Int. Cl. C12M 1/42; C12M 1/00; C12M 1/12; C12M 1/34

(52) U.S. Cl. CPC C12M 35/04; C12M 23/22; C12M 25/02; C12M 41/30; C12M 41/46

(57) ABSTRACT

Provided herein are specially-configured apparatus for maximizing mechanical or acoustic transmission so that a biological material supported by the apparatus reliably experiences a mechanical or acoustic wave that has minimally degraded. In this manner, a biomarker may be reliably monitored and the effect of the mechanical or acoustic wave on the biological material determined. The platform is used to manipulate a cellular interaction with the mechanical or acoustic wave. Related methods of using and making are provided.

### CROSS-REFERENCE TO RELATED APPLICATIONS

This application is a Continuation-in-Part of PCT/US2024/049098, filed Sep. 27, 2024, which claims the benefit of U.S. Provisional Application No. 63/541,663, filed on Sep. 29, 2023, each of which are incorporated by reference herein in their entirety to the extent not inconsistent herewith.

### BACKGROUND OF INVENTION

There is a need in the art for reliably applying acoustic waves to biological material in an in vitro configuration that models, as realistically as possible, the in vivo environment. Provided herein are devices and apparatuses that satisfy this need, including in a manner that maintains good condition of the biological material, before, during and after the acoustic wave introduction. In this manner, the system is compatible with a wide range of applications, including but not limited to, the blood-brain-barrier (BBB).

The BBB prevents over 98% of drugs from entering the brain. Brain tumors such as glioblastomas exhibit altered BBB physiology called the blood-brain tumor barrier (BBTB). Drug permeability of BBTB in the bulk tumor regions is higher than a healthy BBB, which can help drug treatment of the tumor. However, the permeability of BBTB at the peripheral regions of the tumor is similar to a normal BBB, which turns out to still be a major barrier for brain tumor drug delivery. To address the drug delivery issue, multiple approaches have been taken to treat brain disorders such as brain tumors, Alzheimer's and other dementia, Parkinson's disease and bipolar disorder, including viral vector, targeted nanoparticle delivery, gene delivery via exosomes, drug permeability enhancers etc. However, these approaches are still limited by lack of regional specificity, safety concerns, and the amount of drug that can be delivered.

Brain cancer affects 1% of people in the United States and is the 10th leading cause of death. Glioblastomas are the most severe form of brain cancer with an estimated survival rate of less than 6% for individuals aged 55 or older. The need for new treatment methods for glioblastomas is pressing as standard chemotherapeutic medications do not have nearly as much success in the brain as they do in other parts of the body because of the BBB/BBTB. Usually, the best treatment option for patients with glioblastoma is to surgically remove the tumor and if that is not an option, the patient is left with radiation. Brain surgery and radiation are not trivial and can be accompanied by many complications. Sometimes removal of brain tumor and surrounding tissue can result in motor sensory deficits. Moreover, recovery from brain surgery can be lengthy and difficult. There is a demand for an additional technique to the current methods used to treat brain cancer. Clinical trials for a new treatment against glioblastoma using ultrasound (US) and microbubbles are currently underway based on promising pre-clinical data. The technique is described as bubble assisted focused ultrasound (BAFUS) BBB disruption. In other words, when micro/nanobubbles injected into the blood are stimulated by a FUS beam at low frequency (~1 MHz or lower for low scattering by the skull) near a glioblastoma, they cavitate to generate acoustic pressure and physically disrupt the BBB to allow chemotherapeutic drugs to move into the tumor site. Studies showed the disruption can be temporary with BBB recovery observable 4-6 hours after BAFUS. It provides a promising way to deliver large dose of drugs across BBB at targeted locations.

Even though promising, some adverse effects (e.g. failed opening, microhemorrhage) were reported in early clinical trials. There is still a need to better understand the BAFUS BBB opening process to facilitate a safe and effective treatment, which is not fully understood at the cellular and molecular level yet. For example, in this precision medicine era, it is prudent to assume not everyone will respond to BAFUS in the same way. It is therefore useful to understand how the BAFUS parameters affect the BBB disruption and recovery at an individualized level.

To study the process in animals can be expensive. The results can also be misleading due to species differences, and it is challenging to study the process in humans. The current lack of an in vitro model for BAFUS BBB disruption greatly hinders our understanding of the process. Accordingly, there is a need in the art for a platforms that can be used to test how BBB disruption responds to the different BAFUS parameters, with the platform that is acoustically transparent, including to ultrasound, enabling accurate BAFUS power administration, while also being readily imaged and monitored, including throughout a cell culture time course. The platform can be reliably configured for a static or dynamic configuration, depending on whether perfusion and fluid flow to and from the biological material is desired.

### SUMMARY OF THE INVENTION

Provided herein is an apparatus, and related methods, useful for modeling various biological systems in an organ-on-chip platform configuration, such as for testing how BBB disruption responds to the different BAFUS parameters. The apparatus can be acoustically transparent, including to ultrasound, enabling accurate BAFUS power administration. An advantage of the apparatus provided herein is that the optical transparency makes the apparatus easy to image and monitor throughout cell-culture, including BBB-on-Chip, and addresses the need in the art for a relatively low-cost in-vitro model that accurately models the organ and thereby can avoid and/or at least minimize costs associated with animal models and testing.

Although the apparatuses provided herein are useful in a wide range of applications, a preferred use is for the BBB. The BBB is a protective, low permeability tissue structure that separates peripheral blood from the brain. It is composed of a myriad of supporting cells such as pericytes, astrocytes, and microglia. These supporting cells cause tight junctions to form between neighboring brain endothelial cells. Tight junction proteins, occludin and claudin, anchor themselves to the cytoskeletons of two adjacent endothelial cells. This produces a “tight” or leak-free seal between the cells in brain blood vessels. Blood vessels at other tissues/organs have less tight junctions between their endothelial cells with possible fenestration and transcytosis, which allows most molecules in the bloodstreams to move into surrounding tissues. Physiologically, the BBB protects the brain from exposure to toxins and pathogens that may be present in the blood. However, for delivery drugs targeted towards diseases of the brain, the BBB presents a major challenge.

Other useful embodiments for the described apparatus includes, for example, bubble-assisted ultrasound barrier disruption. The apparatus is also compatible with liquid immersion, wherein the apparatus is submerged into a water tank (water is the background material), and the tank surface is covered with sound absorbing materials to minimize any reflection. In this manner, a sound transducer is also submerged into water to deliver sound energy to the acoustic-transparent window area of the device. The acoustic wave can be provided as a beam focused onto the acoustic-transparent window area. The beam can be tilted relative to the acoustic-transparent window normal direction so that any reflection doesn't direct back to the transducer surface. Any top air/water interface can be covered by acoustic absorbing material to minimize any reflection from that interface, as needed. Preferably, the acoustic energy corresponds to ultrasound.

The apparatus channels can be filled with culture media with tubing connected to the inlets and outlets; the culture media can contain material that can interact with US, such as gas bubbles, to generate bubble cavitation and fluidic flows that can disrupt the cellular barrier.

Any of the apparatus described herein are compatible with TransEndothelial Electrical Resistant (TEER) and temperature measurement in the organ-chip (dynamic fluidic control)/transwell (static). Both dynamic and static configurations are compatible with an array configuration for high-throughput assays) that can measure the disruption of a biological barrier, including the BBB cellular barrier, in real-time.

Provided herein is an apparatus for manipulating a cellular interaction with an acoustic wave. Provided herein are methods of making any of the apparatus described herein. Provided herein are methods of using any of the apparatus described herein, including to manipulate a cellular interaction.

Provided herein is an apparatus for manipulating a cellular interaction with a mechanical wave. The apparatus may have a bottom layer forming a first fluidic channel and a top layer forming a second fluidic channel. An intermediate membrane is positioned between the first fluidic channel and the second fluidic channel, wherein the intermediate membrane comprises a receiving surface, wherein the intermediate layer is in fluidic contact with the first fluidic channel and the second fluidic channel. In this manner, the intermediate layer provides one surface to help define the top fluidic channel and another surface that helps define the bottom fluidic channel, with an intermediate layer thickness separating the channels. The first and/or second fluidic channels and/or the receiving surface of the intermediate membrane is configured to support a biological material. For example, the biological material may be supported by the intermediate layer receiving surface, the opposed intermediate layer surface, or both intermediate layer surfaces (e.g., co-culture), or a surface of the fluidic channel. The biological material may be suspended in a liquid in the fluidic channel. The bottom layer, top layer and intermediate membrane are independently configured for mechanical wave transparency. A mechanical wave generator is configured to provide the mechanical wave to the biological material. The mechanical wave generator may be an acoustic wave generator, including an acoustic wave that is in the ultrasound frequency range. A biomarker parameter monitor is configured to monitor a change in one or more biomarkers after provision of the mechanical wave. For an acoustic wave, such as an ultrasound wave, an ultrasonic absorbing material may be positioned so as to reduce unwanted reflection of the acoustic wave.

The apparatus is configured to provide a capability of a dynamic fluid flow and perfusion or a static fluidic configuration. “Dynamic” refers to the ability to control flow rate(s) through one or both fluidic channels, such as via fluidic inlets and outlets. “Static” refers to a lack of fluid flow control, such that the fluidic channels correspond to “reservoirs” that do not fluidically turn over to support biological material.

In an embodiment, an apparatus for manipulating a cellular interaction with an acoustic wave with dynamic fluid control, comprises a bottom layer, a top layer, an intermediate membrane positioned between the top layer and the bottom layer, a biological material, a first fluidic inlet, a first fluidic outlet, an acoustic wave generator, and a biomarker parameter monitor. In an embodiment, the bottom layer comprises a first fluidic channel. In an embodiment, the top layer comprises a second fluidic channel, a second fluidic inlet, and a second fluidic outlet. In an embodiment, the second fluidic inlet is fluidically connected to the second fluidic channel and configured to introduce a second fluid to the second fluidic channel. In an embodiment, the second fluidic outlet is fluidically connected to the second fluidic channel and configured for removal of the second fluid from the second fluidic channel. In an embodiment, the intermediate membrane comprises a receiving surface. In an embodiment, the intermediate membrane is in fluidic contact with the first fluidic channel and the second fluidic channel. In an embodiment, the biological material is positioned inside the first and/or second fluidic channels and/or on the receiving surface of the intermediate membrane. In an embodiment, the first fluidic inlet passes through the top layer and the intermediate membrane, is fluidically connected to the first fluidic channel, and is configured to introduce a first fluid to the first fluidic channel. In an embodiment, the first fluidic outlet passes through the top layer and the intermediate membrane, is fluidically connected to the first fluidic channel and is configured for removal of the first fluid from the first fluidic channel. In an embodiment, the bottom layer, top layer and intermediate membrane are independently configured for acoustic transparency. In an embodiment, the acoustic wave generator is configured to provide the acoustic wave to the biological material. In an embodiment, the biomarker parameter monitor is configured to monitor a change in one or more biomarkers after provision of the acoustic wave.

In an embodiment, the biological material comprises an isolated biological tissue or cultured cells.

In an embodiment, the biological material comprises one or more monolayers of cells.

In an embodiment, the biological material comprises a blood-brain interface or a model of a blood-brain interface.

In an embodiment, the biological material comprises in vitro cultured mammalian cells, including in vitro cultured mammalian cells related to animal and human tissue barriers, such as selected from the group consisting of iPS-derived cells, immortalized human primary cells, Caco-2 cells, MDCK cells, Endo-1 cells, and HBEC-5i cells.

In an embodiment, the biological material is part of a blood-brain-barrier model.

In an embodiment, a portion of the second fluidic channel is positioned above a portion of the first fluidic channel to form a combined overlapping channel volume from the portion of the second fluidic channel with the portion of the first fluidic channel.

In an embodiment, the first fluidic channel is orthogonal to the second fluidic channel. For example, the first fluidic channel is at a 90° to the second fluidic channel, though not necessary intersecting the second fluidic channel. In some embodiments, the first fluidic channel may be vertical, horizontal, or in some other orientation.

In an embodiment, the first fluidic channel has a non-uniform width with a maximum width positioned at a midpoint between the first fluidic inlet and the first fluidic outlet. In an embodiment, the second fluidic channel has a non-uniform width with a maximum width positioned at a midpoint between the second fluidic inlet and the second fluidic outlet. For example, the first and/or second fluidic channel may have the general shape of a rhombus in one cross-section, such that the fluidic inlet is located at or near a vertex of the rhombus and the fluidic outlet is located at or near the opposite vertex.

In an embodiment, the bottom layer and/or top layer comprises polydimethylsiloxane (PDMS), thermoplastic materials such as polystyrene, polycarbonate, PMMA and other cell culture compatible plastic materials.

In an embodiment, the bottom layer and/or top layer comprises a thickness of between 0.001 mm and 10 mm. For example, a thickness between 0.001 mm and 1 mm, between 0.01 mm and 10 mm, between 0.05 mm and 10 mm, between 0.1 mm and 10 mm, between 1 mm and 10 mm, between 0.001 mm and 0.1 mm, or between 0.001 mm and 0.01 mm. For example, a thickness of 0.001 mm, 0.01 mm, 0.05 mm, 0.1 mm, 0.5 mm, 1 mm, 2 mm, 3 mm, 4 mm, 5 mm, 6 mm, 7 mm, 8 mm, 9 mm, or 10 mm.

In an embodiment, the bottom layer further comprises: a first piece of tubing fluidically connected to the first fluidic inlet, and a second piece of tubing fluidically connected to the first fluidic outlet. In an embodiment, the top layer further comprises: a third piece of tubing fluidically connected to the second fluidic inlet, and a fourth piece of tubing fluidically connected to the second fluidic outlet. In an embodiment, each of the first, second, third, and fourth pieces of tubing are configured to be filled with a culture media that supports viability of the biological material.

In an embodiment, the first fluidic channel further comprises a first electrode and a second electrode, and the second fluidic channel further comprises a third electrode and a fourth electrode.

In an embodiment, wherein each of the first and second electrode are independently microfabricated on an inner surface of the first fluidic channel, and each of the third and fourth electrodes are independently microfabricated on an inner surface of the second fluidic channel.

In an embodiment, the intermediate membrane is porous. For example, the intermediate membrane may have a porosity of 1-90%.

In an embodiment, the intermediate membrane comprises a material selected from the group consisting of polyester polytetrafluoroethylene (PETE), polydimethylsiloxane (PDMS), polycarbonate, and parylene.

In an embodiment, the intermediate membrane comprises a thickness of between 0.01 μm and 20 μm. For example, the intermediate membrane comprises a thickness of between 0.01 μm and 10 μm, between 0.05 μm and 20 μm, between 0.1 μm and 20 μm, between 0.1 μm and 10 μm, between 1 μm and 20 μm, or between 1 μm and 10 μm. For example, the intermediate membrane comprises a thickness of 0.01 μm, 0.05 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 12 μm, 14 μm, 16 μm, 18 μm, or 20 μm.

In an embodiment, the intermediate membrane comprises an average pore size of between 0.01 μm and 20 μm. For example, the membrane comprises an average pore size of between 0.01 μm and 10 μm, between 0.05 μm and 20 μm, between 0.1 μm and 20 μm, between 0.1 μm and 10 μm, between 1 μm and 20 μm, or between 1 μm and 10 μm. For example, the membrane comprises an average pore size of 0.01 μm, 0.05 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 12 μm, 14 μm, 16 μm, 18 μm, or 20 μm.

In an embodiment, the bottom layer comprises a bottom viewing frame positioned below the first fluidic channel. In an embodiment, the bottom viewing frame comprises the first fluidic inlet and the first fluidic outlet. In an embodiment, the top layer comprises a top viewing frame positioned above the second fluidic channel. In an embodiment, the top viewing frame comprises the second fluidic inlet and the second fluidic outlet.

In an embodiment, the bottom viewing frame and/or top viewing frame comprises a material selected from the group consisting of: polydimethylsiloxane (PDMS), polystyrene, polycarbonate, poly(methyl methacrylate) (PMMA), and glass.

In an embodiment, the bottom viewing frame and/or top viewing frame comprises a thickness of between 0.1 mm and 10 mm. For example, the bottom viewing frame and/or top viewing frame comprises a thickness of between 0.1 mm and 10 mm or between 1 mm and 10 mm. For example, the bottom viewing frame and/or top viewing frame comprises a thickness of 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, or 10 mm.

In an embodiment, the bottom layer further comprises a first thin layer positioned between the first fluidic channel and the bottom viewing frame, thereby preventing fluid from exiting the first fluidic channel other than at the first fluidic outlet and preventing fluid from entering the first fluidic channel other than at the first fluidic inlet. In an embodiment, the top layer further comprises a second thin layer positioned between the second fluidic channel and the top viewing frame, thereby preventing fluid from exiting the second fluidic channel other than at the second fluidic outlet and preventing fluid from entering the second fluidic channel other than at the second fluidic inlet.

In an embodiment, each of the thin layers comprises a material selected from the group consisting of polydimethylsiloxane (PDMS), parylene, poly-oxydiphenylene-pyromellitimide (Kapton), biaxially oriented polyethylene terephthalate (boPET; Mylar), polystyrene, PMMA, polycarbonate, other plastic materials, ceramic materials, such as glass, silicon nitride. For applications where visual observation is desired, the material may be optically transparent.

In an embodiment, each of the thin layers have a thickness of between 0.01 μm and 100 μm. For example, each of the thin layers have a thickness of between 0.01 μm and 50 μm, between 0.01 μm and 25 μm, between 0.01 μm and 10 μm, between 0.01 μm and 1 μm, between 0.1 μm and 100 μm, between 0.5 μm and 100 μm, between 1 μm and 100 μm, or between 10 μm and 100 μm. For example, each of the thin layers have a thickness of 0.01 μm, 0.05μ, 0.1 μm, 0.2 μm, 0.3 μm, 0.4 μm, 0.5 μm, 1 μm, 2μ, 3μ, 4μ, 5μ, 10 μm, 20 μm, 30 μm, 40 μm, 50 μm, 60 μm, 70 μm, 80 μm, 90 μm, or 100 μm.

In an embodiment, the acoustic wave generator is configured to introduce an acoustic waveform to the biological material.

In an embodiment, the acoustic wave generator is further configured to: focus the acoustic waveform on the combined overlapping channel volume; and tilt from a position normal to the layers to reduce acoustic waves reflected from the layers back to the acoustic wave generator.

In an embodiment, the apparatus further comprises gas bubbles within a fluid, wherein the gas bubbles are configured to cavitate upon interaction with the acoustic waveform and thereby interact with the biological material.

In an embodiment, the acoustic waveform comprises acoustic pulses with an acoustic frequency selected from the range of 100 kHz to 20 MHz. For example, an acoustic frequency selected from the range of 200 kHz to 20 MHz, 500 kHz to 20 MHz, 1 MHz to 20 MHz, 10 MHz to 20 MHz, 100 kHz to 10 MHz, 100 kHz, to 1 MHz, 100 kHz to 500 kHz, or 100 kHz to 250 kHz. For example, an acoustic frequency of 100 kHz, 200 kHz, 250 kHz, 300 kHz, 400 kHz, 500 kHz, 750 kHz, 1 MHz, 5 MHz, 10 MHz, 15 MHz, or 20 MHz. In an embodiment, each pulse comprises a pulse duration selected from the range of 100 ns to 10 s. For example, a pulse duration selected from the range of 100 ns to 5 s, 100 ns to 10 us, 100 ns to 1 ms, 100 ns to 100 ms, 100 ns to 1 s, 10 us to 10 s, 500 us to 10 s, 1 ms to 10 s, 10 ms to 10 s, 1 ms to 1 s, 1 ms to 10 s, 10 ms to 10 s, 100 ms to 10 s, or 1 s to 10 s. For example, a pulse duration of 100 ns, 200 ns, 250 ns, 500 ns, 750 ns, 1 us, 10 us, 20 us, 50 us, 1 ms, 2 ms, 5 ms, 10 ms, 20 ms, 50 ms, 100 ms, 200 ms, 500 ms, 1 s, 2 s, 5 s or 10 s. In an embodiment, the pulses are repeated with a frequency between 0.1 to 10 Hz. For example, a frequency of 0.1, 0.2, 0.3, 0.4, 0.5, 0.6, 0.7, 0.8, 0.9, 1, 1.5, 2, 3, 5, 7, 10 Hz. In an embodiment, the number of pulses range from 1 to 1000. For example, 1, 2, 5, 10, 20, 40, 60, 80, 100, 120, 150, 170, 200, 250, 300, 400, 600, 800, 1000 pulses.

In an embodiment, the acoustic waveform comprises a continuous acoustic wave with an acoustic frequency selected from the range of 100 kHz to 20 MHz. For example, an acoustic frequency selected from the range of 200 kHz to 20 MHz, 500 kHz to 20 MHz, 1 MHz to 20 MHz, 10 MHz to 20 MHz, 100 kHz to 10 MHz, 100 kHz, to 1 MHz, 100 kHz to 500 kHz, or 100 kHz to 250 kHz. For example, an acoustic frequency of 100 kHz, 200 kHz, 250 kHz, 300 kHz, 400 kHz, 500 kHz, 750 kHz, 1 MHz, 5 MHz, 10 MHz, 15 MHz, or 20 MHz. In an embodiment, the acoustic waveform comprises an ultrasound waveform. In an embodiment, the acoustic wave generator is an ultrasound generator.

In an embodiment, the biomarker parameter comprises a parameter that can be affected by acoustic waves. For example, the parameter may be increased, decreased, or otherwise altered due to the presence or absence of acoustic waves.

In an embodiment, the biomarker parameter comprises a measure of cellular barrier disruption, a protein, mRNA, a polynucleotide, cell morphology, and/or cell viability.

In an embodiment, the biomarker parameter comprises a measure of cellular barrier disruption selected from the group consisting of a permeability parameter of the biological material, a temperature parameter of the biological material, and a transendothelial electrical resistance (TEER) parameter of the biological material. In an embodiment, the TEER can be measured by a 4-electrode system with a pair of electrodes for conducting current and a pair of electrodes for measuring voltage drop, or by a 2-electrode system using impedance spectroscopy.

In an embodiment, the apparatus further comprises a container configured to hold a fluid. In an embodiment, at least a portion of a surface(s) of said container is covered with an ultrasound absorbing material. For example, the ultrasound absorbing material may only allow less than 20%, less than 10%, less than 5%, less than 1%, less than 0.1%, or less than 0.01% of an ultrasound to be reflected by the material.

In an embodiment, the container is filled with water. In an embodiment, the bottom layer, the top layer, the intermediate membrane, and the biological material are submerged in said water.

In an embodiment, the acoustic wave generator is submerged in said water.

In an embodiment, an air/water interface is covered with an ultrasound absorbing material.

In an embodiment, the ultrasound absorbing material is configured to reduce reflections of the acoustic waveform that is an ultrasonic waveform.

Provided are methods of making any apparatus described herein. For example, provided is a method of making an apparatus for the study of cellular interaction with ultrasound with a dynamic fluid flow control, comprising, providing a bottom layer, providing a top layer, providing an intermediate membrane, providing a first fluidic inlet, providing a first fluidic outlet, fluidically contacting the intermediate membrane with the first fluidic channel, fluidically contacting the intermediate membrane with the second fluidic channel, providing a biological material to the first fluidic channel and/or the second fluidic channel and/or the receiving surface of the intermediate membrane, acoustically stimulating a biological material with an acoustic wave using an acoustic wave generator, and monitoring a change in one or more biomarkers after acoustic stimulation using a biomarker parameter monitor. In an embodiment, the bottom layer comprises a first fluidic channel configured to be in fluidic contact with an intermediate membrane. In an embodiment, the bottom layer is configured for acoustic transparency. In an embodiment, the top layer comprises: a second fluidic channel configured to be in fluidic contact with said intermediate membrane, a second fluidic inlet, allowing the introduction of a fluid into the second fluidic channel, and a second fluidic outlet, allowing the flow of a fluid out of the second fluidic channel. In an embodiment, the top layer is configured for acoustic transparency. For example, the top layer and/or the bottom layer may allow greater than 50%, greater than 60%, greater than 70%, greater than 80%, greater than 90%, greater than 95%, greater than 99%, or greater than 99.9% of acoustic waveforms to pass through, less than 20%, less than 10%, less than 5%, less than 1%, less than 0.1%, less than 0.01% to be reflected.

In an embodiment, the biological material comprises an isolated biological tissue or cultured cells.

In an embodiment, the biological material comprises one or more monolayers of cells.

In an embodiment, the biological material comprises a blood-brain interface or a model of a blood-brain interface.

In an embodiment, the biological material comprises in vitro cultured mammalian cells, including in vitro cultured mammalian cells related to animal and human tissue barriers, such as selected from the group consisting of iPS-derived cells, immortalized human primary cells, Caco-2 cells, MDCK cells, Endo-1 cells, and HBEC-5i cells.

In an embodiment, providing the bottom layer and/or top layer comprises injection molding.

In an embodiment, said injection molding comprises soft lithography.

In an embodiment, providing the intermediate membrane comprises: providing a thin layer of material; and laser cutting the intermediate membrane from the thin layer of material.

In an embodiment, the bottom and top layers each comprise polydimethylsiloxane (PDMS). In an embodiment, the intermediate membrane comprises a material selected from the group consisting of polyester polytetrafluoroethylene (PETE), polydimethylsiloxane (PDMS), polycarbonate, and parylene.

In an embodiment, the method further comprises: attaching a first thin layer to the first fluidic channel; attaching a bottom viewing frame to the first thin layer; attaching a second thin layer to the second fluidic channel; attaching a top viewing frame to the second thin layer.

In an embodiment, the bottom layer comprises a bottom viewing frame; the top layer comprises a top viewing frame; and the bottom viewing frame and/or top viewing frame are formed by injection molding.

In an embodiment, the method relates to injection molding. In an embodiment, the method comprises soft lithography.

In an embodiment, each of the first and second thin layers are formed by: providing a silicon wafer; cleaning the silicon wafer; applying a photoresist to the silicon wafer; curing the photoresist to the silicon wafer; applying a material to the cured photoresist; and curing the material.

In an embodiment, applying the photoresist to the silicon wafer comprises spin coating. In an embodiment, applying the material to the cured photoresist comprises spin coating.

In an embodiment, the bottom viewing frame and the top viewing frame each independently comprise a material selected from the group consisting of polydimethylsiloxane (PDMS), polystyrene, polycarbonate, poly(methyl methacrylate) (PMMA), and glass. In an embodiment, the first thin layer and the second thin layer each independently comprise a material selected from the group consisting of polydimethylsiloxane (PDMS), parylene, poly-oxydiphenylene-pyromellitimide (Kapton), biaxially oriented polyethylene terephthalate (boPET; Mylar), polystyrene, PMMA, polycarbonate, other plastic materials, or any ceramic materials, such as glass and silicon nitride.

In an embodiment, a method of evaluating a cellular interaction with an acoustic wave, comprises providing an apparatus described above, introducing the first fluid into the first fluidic channel, introducing the second fluid into the second fluidic channel, introducing the second fluid into the second fluidic channel, and monitoring a biomarker parameter of the biological material, thereby evaluating the cellular interaction with ultrasound. In an embodiment, the introduced first fluid and second fluid are provided to the biological material.

In an embodiment, applying an acoustic wave to the biological material comprises applying acoustic pulses to the biological material with an acoustic frequency selected from the range of 100 kHz to 10 MHz. For example, an acoustic frequency is selected from the range of 100 kHz to 20 MHz. For example, an acoustic frequency selected from the range of 200 kHz to 20 MHz, 500 kHz to 20 MHz, 1 MHz to 20 MHz, 10 MHz to 20 MHz, 100 kHz to 10 MHz, 100 kHz, to 1 MHz, 100 kHz to 500 kHz, or 100 kHz to 250 kHz. For example, an acoustic frequency of 100 kHz, 200 kHz, 250 kHz, 300 kHz, 400 kHz, 500 kHz, 750 kHz, 1 MHz, 5 MHz, 10 MHz, 15 MHz, or 20 MHz. In an embodiment, each pulse comprises a pulse duration selected from the range of 100 ns to 10 s. For example, a pulse duration selected from the range of For example, a pulse duration selected from the range of 100 ns to 5 s, 100 ns to 10 us, 100 ns to 1 ms, 100 ns to 100 ms, 100 ns to 1 s, 10 us to 10 s, 500 us to 10 s, 1 ms to 10 s, 10 ms to 10 s, 1 ms to 1 s, 1 ms to 10 s, 10 ms to 10 s, 100 ms to 10 s, or 1 s to 10 s. For example, a pulse duration of 100 ns, 200 ns, 250 ns, 500 ns, 750 ns, 1 us, 10 us, 20 us, 50 us, 1 ms, 2 ms, 5 ms, 10 ms, 20 ms, 50 ms, 100 ms, 200 ms, 500 ms, 1 s, 2 s, 5 s or 10 s. In an embodiment, the pulses are repeated with a frequency between 0.1 to 10 Hz. For example, a frequency of 0.1, 0.2, 0.3, 0.4, 0.5, 0.6, 0.7, 0.8, 0.9, 1, 1.5, 2, 3, 5, 7, 10 Hz. In an embodiment, the number of pulses range from 1 to 1000. For example, 1, 2, 5, 10, 20, 40, 60, 80, 100, 120, 150, 170, 200, 250, 300, 400, 600, 800, 1000 pulses.

In an embodiment, wherein applying ultrasound to the biological material comprises applying a continuous acoustic wave to the biological material with an acoustic frequency selected from the range of 100 kHz to 20 MHz. For example, an acoustic frequency selected from the range of 200 kHz to 20 MHz, 500 kHz to 20 MHz, 1 MHz to 20 MHz, 10 MHz to 20 MHz, 100 kHz to 10 MHz, 100 kHz, to 1 MHz, 100 kHz to 500 kHz, or 100 kHz to 250 kHz. For example, an acoustic frequency of 100 kHz, 200 kHz, 250 kHz, 300 kHz, 400 kHz, 500 kHz, 750 kHz, 1 MHz, 5 MHz, 10 MHz, 15 MHz, or 20 MHz.

In an embodiment, monitoring the biomarker parameter of the biological material comprises: monitoring the biomarker parameter of the biological material with respect to a molecular tracer.

In an embodiment, the molecular tracer comprises 70 k-Dextran-TMR or Lucifer Yellow.

In an embodiment, the method further comprises the step of co-culturing at least two different biological cells. For example, the method may comprise the step of co-culturing 2 or more types of cells, such as between 2 and 10, 2 and 5, 2 and 4, 2 and 3 or 2.

In an embodiment, the monitoring step comprises measuring an electrical parameter of the biological material with a plurality of electrodes. For example, the monitoring step may comprises measuring an electrical parameter of the biological material with a pair of electrodes in each of the fluidic channels, such as with four electrodes. The monitoring may be via TEER measurement, and may include use of hydrogels in a basolateral compartment, including hydrogels to better facilitate permeability characterization. The hydrogel is also useful for inclusion of vessels within the hydrogel, including vessels, all within the ultrasound transparent compartment. This can provide better modeling of BBB with attendant improvement in drug screening under ultrasound and gas bubble stimulus to model disruption of the BBB and assess ideal conditions for particular drugs to ensure brain access.

### BRIEF DESCRIPTION OF THE DRAWINGS

FIG. 1A is a schematic illustration of one embodiment of an apparatus for manipulating a cellular interaction with an acoustic wave. The schematic is a cross-section illustrating two pairs of conducting electrodes useful for measuring a biomarker that is an electrical resistance of the biological tissue. FIG. 1B illustrates a biological material on the receiving surface of the intermediate membrane in the second fluidic channel. For simplicity and ease of illustration, only a small portion of the receiving surface supports the biological material. The apparatus is, of course, compatible with a layer of cells that can cover up to the entire available surface of the intermediate membrane. FIG. 1C illustrates a biological material in the first fluidic channel.

FIG. 2 is a schematic illustration of one embodiment of an apparatus for BAFUS for use in the BBB-on-chip.

FIGS. 3A-3B are images of injection molding of US-transparent chip PDMS frames. FIG. 3A is an image of the two pieces of the mold, the frame reservoir (top) and the injection inlet cover (bottom). FIG. 3B is an image of the assembled mold being injected with uncured PDMS at a single frame location.

FIG. 4 is an illustration of thin PDMS membrane fabrication. AZ1512 and uncured PDMS were coated on the Si wafer consecutively using different spin coating and baking conditions.

FIG. 5 is an image of US-transparent chip assembly. On the left is the assembly guide used on its own without an added device. The blunt ended needles go through the two alignment holes on each frame as demonstrated on the right side of the image with the complete device placed on the guide frame by frame.

FIGS. 6A-6C are images and illustrations of needle hydrophone characterization setup. FIG. 6A shows the US transparency characterization system. The US-transparent window of the device could be perpendicular (FIG. 6A) or 31° to the transducer-hydrophone axis (FIG. 6B). FIG. 6C shows a schematic drawing of the characterization setup.

FIGS. 7A-7C are images and illustrations of the device geometry. FIG. 7A is a diagram of exemplary dimensions of the apparatus, FIG. 7B is a COMSOL generated physics-controlled mesh of the 2D plane of a single channel, and FIG. 7C is a physics-controlled mesh of the whole inner region of the device's channels.

FIGS. 8A-8C are a diagram of exploded Organ-on-Chip and real images for the components and assembled device. FIG. 8A is a schematic showcasing then layers of the PDMS and PETE constructed Organ-on-Chip. FIG. 8B is a real image of the surrounding clamp (1/2), the 3D printed barb to needle conversion piece (3) and the PDMS device (4). FIG. 8C is a real image of the complete device with a size of about 2.54 cm (1 inch).

FIG. 9 is an image of a leak-free device with colored dyes flowing through both channels of the US-transparent chip. Orange dye was flowed through the bottom channel and pink dye was flowed through the top channel.

FIG. 10 is a thin layer model pulled from Waves of Layered Media (20).

FIGS. 11A-11D are solutions to model reflection through a thin PDMS membrane (FIGS. 11C-11D) and a thin PETE membrane (FIGS. 11A-11B). The wave-like graphs at the top of the figure demonstrate the reflection solutions at a large range of membrane thicknesses (0-2 mm) while the lower linear graphs are of a smaller 50 μm thickness range.

FIGS. 12A-12B are plots of 2D velocity (FIG. 12A) and pressure gradient (FIG. 12B) profile solutions to a laminar flow model of the y-plane of an individual channel based on the mesh generated and shown in FIGS. 7B-7C. Fluid is flowing from the left to right where the inlet and outlet are the leftmost and rightmost arks of the geometry.

FIGS. 13A-13B are plots of 3D velocity (FIG. 13A) and pressure gradient (FIG. 13B) profile solutions to a laminar flow model of the y-plane of an individual channel based on the mesh generated and shown in FIG. 7B-7C. The inlets and outlets of this geometry are the bottom-most and top-most upwards projecting cylinder faces respectively.

FIGS. 14A-14B are solutions to the transport of diluted species model for the Organ-On-Chip device at two different membrane diffusivities. The bottom channel is a set 100 μM concentration and flow moves from left to right at an initial 1 mm/s velocity with an inlet concentration of 0 μM.

FIGS. 15A-15B is data showing that bubbles were considered detected when sub- and super-harmonic signals emerged. FIG. 15A demonstrates the background signal when the device is filled with PBS. FIG. 15B demonstrates the signal output to the oscilloscope when the top channel is filled with activated bubble solution and the bottom is filled with PBS.

FIG. 16 is a peak enhancement curve for a 0.5 MHz signal produced by stimulated nanobubbles in a 100× dilution DBPC primary lipid recipe. The definitions of signal enhancement as the curve peak height and half-life as the time for the enhancement to decrease by 6 dB are also depicted.

FIGS. 17A-17B are half-life (FIG. 17A) and signal enhancement characterization (FIG. 17B) of DPPC and DBPC primary lipid recipes at 100× and 500× dilutions.

FIG. 18 is data showing DPBC primary lipid recipe nanobubbles exhibited longevity when diluted 100× under constant 15 ml/hr flow.

FIG. 19 is a transmission electron microscopy image of a nanobubble produced by a 100× diluted recipe using DBPC as the primary lipid.

FIG. 20 is average TEER measurements for monolayers of a panel of cell types including HBEC-5i, Caco-2, and an in-house differentiated brain endothelial cell type. All measurements were made after 24 hours with the exception of the differentiated cell type for which a 48 hour measurement was also made. The TEER values displayed in this figure were not standardized to the membrane and coated controls, which is why the values for the controls are also provided.

FIGS. 21A-21C are permeability measurements for a panel of cell types grown in transwells with 70 kDa Dextran-TMR (left) and Lucifer Yellow (right) permeability measurements of in-house differentiated brain endothelial cells, HBEC-5i cells, and Caco-2 cells, respectively. A paired, equal variance T-test was performed for all figures where **** indicates a p-value<0.0005, * indicates a p-value<0.5, and n.s. indicates the p-value was >0.05.

FIG. 22 is TEER measurements over 72 hours for 150 k and 200 k initial transwell seedings of Caco-2 cells. These data were standardized against an average coated and uncoated control data set.

FIGS. 23A-23B are Permeability Lucifer yellow (FIG. 23A) and 70 k-Dextran-TMR (FIG. 23B) in a transwell system with control membranes or membranes layered with Caco-2 cells. A paired, equal variance T-test was performed for all figures where **** indicates a p-value<0.0005 and n.s. indicates the p-value was >0.05.

FIGS. 24A-24C are contrast images of Caco-2 culture in the US-transparent Chip at baseline (FIG. 24A), post-BAFUS (FIG. 24B), and post-recovery (FIG. 24C). Images were taken at 10× magnification, and the white scale bar is 100 μm in all three images.

FIG. 25 is baseline, post-BAFUS, recovery, and control permeability measurements for Caco-2 cells grown in the US-transparent chip. Baseline measurements were taken 24 hours after initial cell seeding. BAFUS was administered immediately following the 24-hour baseline permeability measurement, which was followed by the post-BAFUS permeability measurement. The post-recovery measurement was taken 24 hours after BAFUS treatment. The control measurements were made in uncoated devices with no cell culture. The baseline, post-BAFUS, and post-recovery measurements were averages from two devices. The control data were averages from 6 runs.

FIG. 26A is a schematic illustration of one embodiment of an apparatus for FUS for use in the BBB-on-chip. FIG. 26B-26C are an illustration and an image of a needle hydrophone characterization setup. FIG. 26D is a graph showing the change in peak negative pressure in response to changes in waveform generator voltage.

FIG. 27A are images of nanobubbles, including a TEM image of a nanobubble with FIGS. 15A-15B related graphs showing the harmonic peaks obtained with a control PBS solution, and the super/subharmonic peaks obtained with a nanobubble solution, respectively. FIG. 27B is a plow showing DPBC primary lipid recipe nanobubbles exhibited longevity when diluted 100× under constant 15 ml/hr flow.

FIG. 28 illustrates co-cultured BBB cells yield durable and physiological TEER barriers over time.

FIGS. 29A-29D show a hypoxic culture of two iPSC-derived first passage BMVECs fosters robust EC tight junctions (ZO-1, green) and differentiation (CD31, red). FIGS. 29A-29B are obtained using Model 1. FIGS. 29C-29D are obtained using Model 2.

FIG. 30 shows BMVEC marker CD31 and tight junction proteins: occludin and Claudin-5 expression by long term endothelial culture (HBEC-5i) and iPSC Model 1-derived first passage BMVECs propagated under hypoxic conditions.

FIG. 31 shows Claudin-5 SNVs in the initial inventory of iPSC lines for BBB investigation. Exome Seq data from six iPSC lines (3 Female, 3 Male) were queried for SNVs of CLDN5. There are shared and unique SVNs across the models. On-chip BBB performance, CLDN5 expression, and vulnerability to BAFUS in these models will initiate hypothesis development on the role of genotype on BAFUS vulnerability phenotype.

FIG. 32 compares the NPpeak ratio when the FUS is at 0 degrees and at 31 degrees.

FIG. 33 is a schematic and flow chart of the components used in the ultrasound delivery system. Text indicates the component name, with a representative manufacturer and model number.

FIG. 34 is a labeled image of the components in the water bath submerging the transducers. The water level is filled to just cover the bottom transducer surfaces.

FIG. 35 is a diagram depicting the focus region of each of the transducers, their angles, and where they overlap in the 6 mm view hole of the device.

FIG. 36 shows confluent HBMEC-5i cells grow in adherent culture in DMEM+10% FBS+10%+1% ECGS (endothelial cell growth supplement).

FIGS. 37A-37B show HBMEC-5i cells that are stained with DAPI (blue) and key tight junction markers: ZO-1 (FIG. 37A, green) and occludin (FIG. 37B, green). Composite z-stack images are taken on confocal microscopy.

FIG. 38 is a graph showing an ultrasound thin membrane impedance physics model of the reflection coefficient change with PDMS membrane thickness at different angle of incidence.

FIG. 39 is graphs showing that the device is ultrasound transparent (left) and the difference in permeability between the transwell and the BBB-On-Chip.

FIG. 40 is a graph showing the peak stability of 0.5 MHz signal enhancement vs. time after 0.5 mL injection.

FIG. 41 is a series of images showing the steps of the nanobubble fabrication process.

FIG. 42 is an image of a nanobubble taken using cryo-TEM.

FIG. 43 is a flow chart showing an embodiment of a method of making an apparatus for the study of cellular interaction with ultrasound with a dynamically controllable fluid flow in a fluidic channel via inlet and outlet.

FIG. 44 is flow chart showing an embodiment of a method of evaluating a cellular interaction with an acoustic wave.

FIG. 45A schematically illustrates a fluidic configuration in a trans-well format to facilitate high throughput screening with the left panel having a well with a thin membrane and the right panel having an acoustic wave absorption material. FIG. 45B illustrates a configuration without fluidic inlets or outlets for static flow configuration, with electrodes in contact with the top fluidic channel and another pair of electrodes with the bottom fluidic channel. The acoustic absorption material is positioned further away from the intermediate membrane than the embodiment of FIG. 45A. FIG. 45C illustrates an embodiment where the acoustic wave comes from the top cap portion, i.e. the acoustic generator is fluidically coupled to the top thin layer (both are in contact with a fluid, e.g. water), with the acoustic absorbing material (fluidically coupled to the thin layer of the bottom channel) positioned at the bottom of the acoustic wave exit. The acoustic absorbing material can be in direct contact with the top fluidic channel, close or far away from the intermediate membrane.

FIG. 46A is a schematic exploded illustration of an apparatus that is ultrasonically transparent, and having a basolateral compartment for containing a hydrogel (top panel). The bottom panel is a photograph of an assembled apparatus, including a f-PCB TEER cable. Acoustic pressure loss is determined to be less than 2% (data not shown). FIG. 46B is a plot of TEER measurement (rt-TEER) (left vertical axis) and temperature measurement (right vertical axis) of Caco-2-chip up to 72 hrs from cell seeding. FIG. 46C are subharmonic and broadband cavitation dose curves from Definity-like microbubbles. FIG. 46D TEER of iBMEC barrier reached >5,500 ohm-cm². FIG. 46E preliminary FUS opening of a Caco-2-chip barrier as reflected by permeability.

### DETAILED DESCRIPTION

Provided herein is an apparatus for manipulating cellular interaction with a mechanical wave, including an acoustic wave. A biological material provides the cells. “Biological material” is used broadly herein and can include isolated biological tissue, cultured cells, and the like. The biological material may include one or more monolayers of cells. The biological material may be a blood-brain interface, including a biological model of a blood-brain interface. The biological material may be a co-culture comprising two or more cell types or tissues. For example, one portion of the co-culture may be positioned in a first fluidic channel and a second portion of the co-culture in a second fluidic channel. The porous intermediate membrane may define surfaces of each of the two channels, thereby facilitating cross-talk. The separate inlets and outlets to each of the fluidic channels allows different culture media and gas to be applied to the different co-culture portions.

Mechanical wave is used broadly herein to refer to a stimulus that is able to travel or transmit through regions of the device to interact with a biological material. It may arise from an impulse force, sudden mechanical force, or acoustic disturbance, with a resultant wave that travels through the apparatus and interacts with a biological material. The mechanical force may model a concussive type force to thereby study the influence of that sort of force on biological material. In an embodiment, a mechanical force that is an acoustic wave, including an ultrasound wave, it utilized.

“Acoustic transparency” refers to the ability of at least a portion of the apparatus to pass an acoustic wave to the biological material without substantial reflection and attenuation. Acoustic transparency may refer to less than 20% reflection, less than 10% reflection, less than 5% reflection, less than 1% reflection, less than 0.1% reflection, or less than 0.01% reflection, and less than 50% attenuation, less than 75% attenuation, less than 90% attenuation, and less than 95% attenuation, at a user-defined wavelength or over a wavelength range. Depending on the application of interest, the wavelength may correspond to an ultrasonic wavelength (e.g., above human hearing) including, for example, above 20 kHz, or between about 100 kHz and 20 MHz. In an embodiment, desirable acoustic transparency is achieved by special positioning of windows (e.g., minimal absorbent material) in combination with thin layers through which the acoustic wave must travel. Specific thickness values will vary with acoustic frequency. A preferable thickness of a layer characterized as “thin” is less than 100 μm, less than 20 μm, less than 1 μm (e.g., “nanometer” sized) and less than 0.1 μm.

“Acoustic generator” is used broadly herein and reflects that there are many different instruments capable of generating radiofrequency soundwaves of a desired range. A preferred embodiment is an ultrasonic generator that generates ultrasound, including corresponding to the above ranges for “ultrasonic wavelength”. The term “generator” is intended to include ancillary components so that the generated acoustic wave is reliably provided to the biological material, such as a coupling medium, absorbing materials and other components to minimize or avoid unwanted interfaces, such as air/liquid that would otherwise adversely impact ultrasound clarity.

“Biomarker parameter” is used broadly herein to refer to any number of parameters that can be affected by ultrasonic stimulation. Examples include, but are not limited to, cellular morphology, viability, functional property of a cell, including that impacts mass transfer to a cell (e.g., nutrient and drug delivery or uptake by a cell), a measure of cellular barrier disruption (e.g., permeability), electrical resistance including a transendothelial electrical resistance, temperature, a protein, mRNA, a polynucleotide, and the like, depending on the application of interest. A “biomarker parameter monitor” is a device that provides a measure of the value of the biomarker parameter. Depending on the biomarker of interest, it may be electrical in nature (e.g., electrodes measuring resistance across a surface), optical (measure a fluorescent marker or label), a western blot (to detect presence/absence of a protein), a PCR device (detect presence/absence of a polynucleotide sequence), etc.

Components being “fluidically connected” as used herein means that a fluid is able to flow from one component to another. Components that are fluidically connected are not necessarily in physical connect, though they may be.

“Microfabrication” or “microfabricated” as used herein means the fabrication of a device, component, structure, or material at a scale of 1 mm or smaller. Microfabrication may comprise the use of thin films. For example, microfabrication may comprise thin film deposition, including thermal oxidation, chemical vapor deposition, physical vapor deposition, sputtering, evaporative deposition, electron beam physical vapor deposition, or epitaxy. Microfabrication may comprise patterning, including photolithography or shadow masking. Microfabrication may comprise etching, including wet etching, plasma etching, reactive-ion etching, deep reactive-ion etching, wet etching, or chemical etching. Microfabrication may comprise nanofabrication.

“Ultrasound” as used herein means an acoustic wave with a frequency greater the 20 kHz. For example, an ultrasound frequency may be selected from the range of 20 kHz to 200 MHz, 20 kHz to 2 MHz, or any subrange thereof. For example, the frequency may be selected from the range of 200 kHz to 20 MHz, 500 kHz to 20 MHz, 1 MHz to 20 MHz, 10 MHz to 20 MHz, 100 kHz to 10 MHz, 100 kHz, to 1 MHz, 100 kHz to 500 kHz, or 100 kHz to 250 kHz. For example, an ultrasound frequency may be 100 kHz, 200 kHz, 250 kHz, 300 kHz, 400 kHz, 500 kHz, 750 kHz, 1 MHz, 5 MHz, 10 MHz, 15 MHz, or 20 MHz.

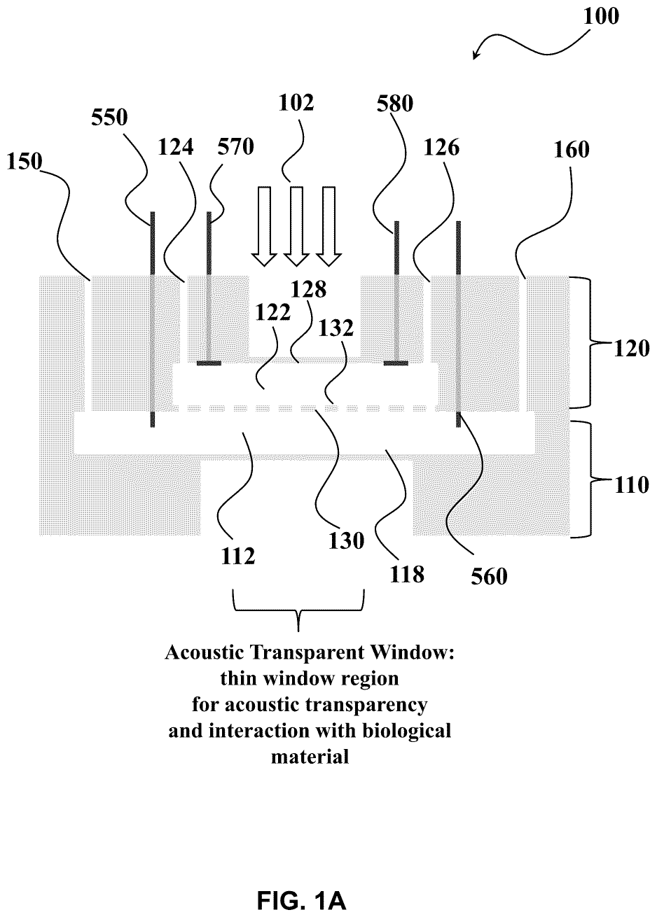

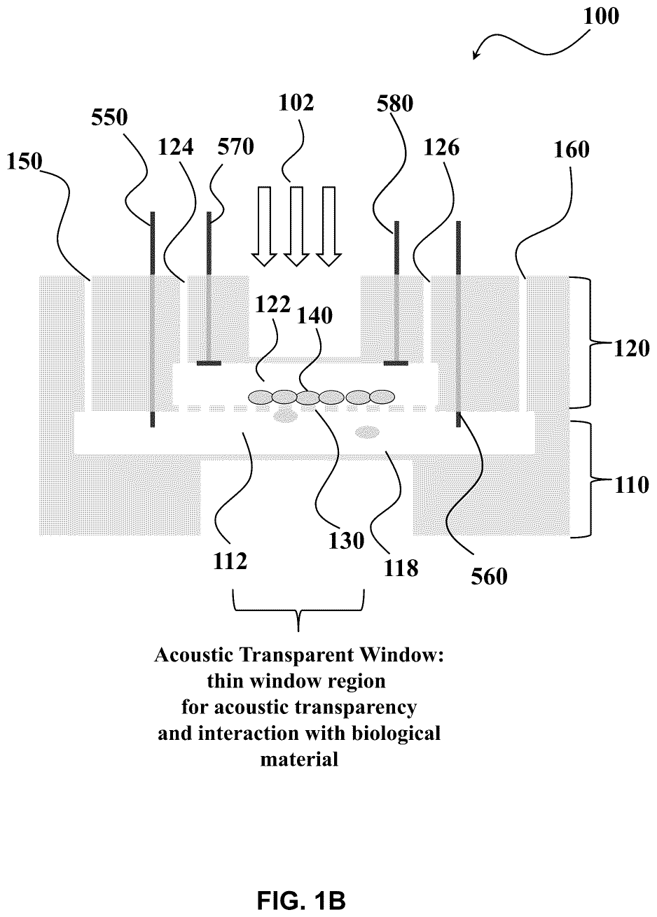

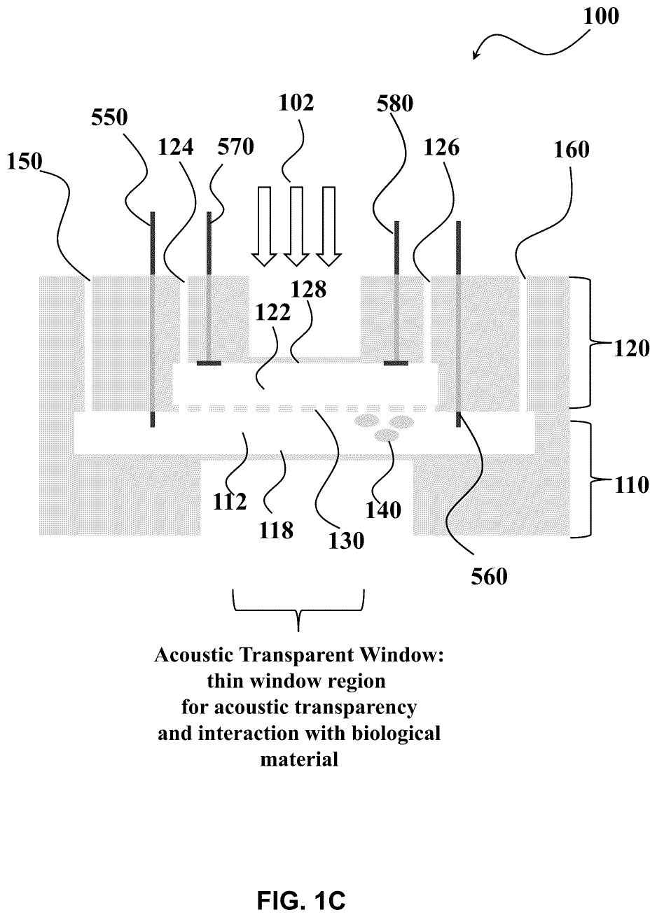

One example of an apparatus is provided in FIG. 1A-1C, which is a 2D cross-sectional view of the apparatus. Illustrated components include: One top fluidic compartment/channel 120, with an inlet 124 and an outlet 126; One bottom fluidic compartment 110, with an inlet 150 and an outlet 160; A porous thin middle (e.g., “intermediate”) layer 130 separating the overlapping areas of the top and bottom compartments, serving as part of the bottom surface of the top compartment and part of the top surface of the bottom apartment. The top covering material for the top compartment is thin; the bottom covering material for the bottom compartment is thin. In this context, “thin” refers to a thickness that results in suitable transmission (=Aout/A n) and reflection (=Areflection/Ain), where A is acoustic wave amplitude, reflection is the reflected amplitude, and out is the output and in is the incident acoustic wave amplitude. Depending on the application of interest, the reflection may be at most 20%, at most 10%, at most 5%, at most 1%, at most 0.1%, at most 0.01%; transmission may be at least 50%, at least 75%, at least 90%, at least 95% or at least 99%, at a user-selected frequency suitable for cellular interaction. FIG. 1B illustrates a co-culture of biological material 140 (e.g., material in first channel and material in second channel, including supported by intermediate membrane 130 and/or freely suspended in the channel(s)).

The overlapping of the top thin region, middle compartment overlapping area, and the bottom thin region comprises a window for acoustic wave interaction with biological materials inside the two compartments with minimal acoustic absorbance/reflection/scattering/interference or maximal acoustic transmission (e.g., “acoustically transparent”).

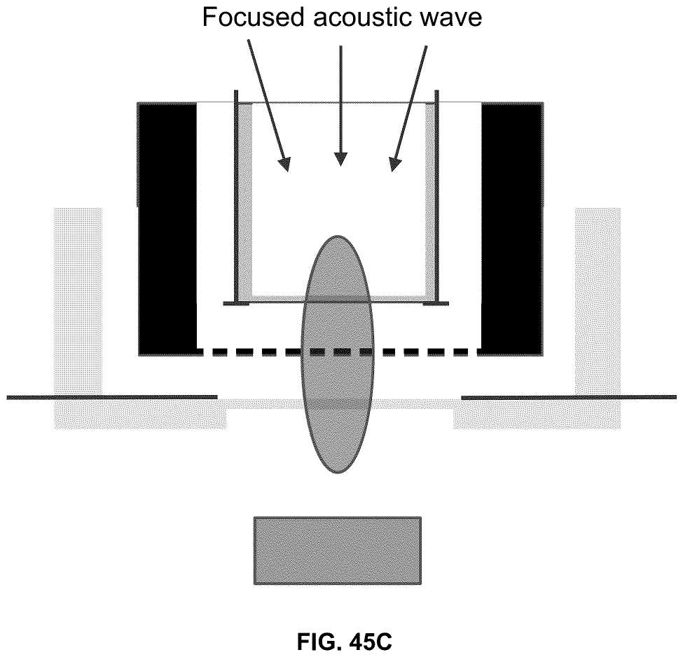

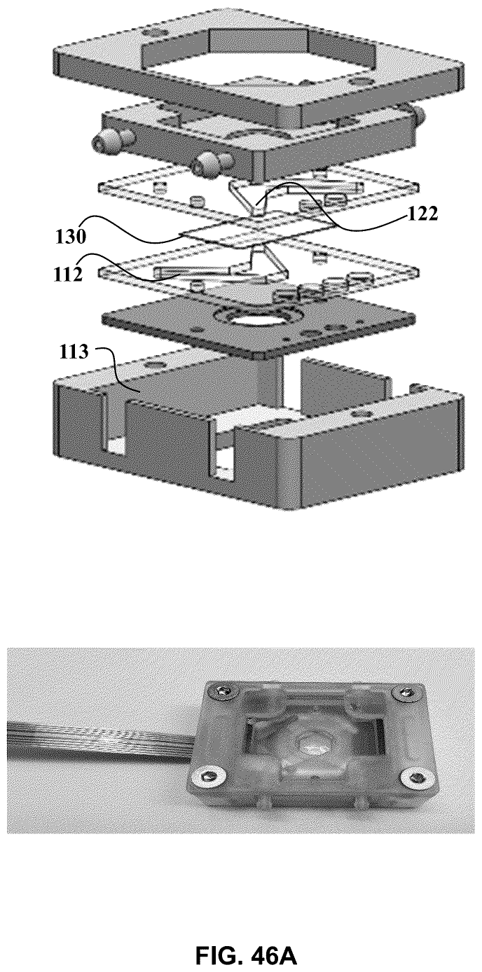

In this example, the biomarker parameter monitor 180 is illustrated with conducting electrodes 550, 560, 570, 580 (see, e.g., FIGS. 1A-1C FIG. 45A-45C) useful for assessing a biomarker that is an electrical resistance. For example, the biomaterial 140 may be an endothelial cell layer and the biomarker transendothelial resistance with the monitor a transendothelial resistance (TEER) electrode. The TEER electrodes may include:

Two conducting electrodes 550, 560 positioned into the top compartment (could be on compartment surface to into the compartment space) to make contact with the fluid inside the compartment. They are on outside and separated by the acoustic window area.

Two conducting electrodes 570, 580 positioned into the bottom compartment (could be on a compartment surface and/or into the compartment space) to make contact with the fluid inside the bottom compartment. They are on outside and can be separated a separation distance that is greater than or equal the acoustic window area distance, for example.

Conducting wires connect the electrodes to the outside of the device.

Another embodiment of an apparatus is provided in FIG. 45A-45C. FIG. 45A illustrates an apparatus 100′ that is a transwell-like device arrayed into a well-plate format for high throughput screening. This aspect may be referred to as a “static configuration” in that it is not necessary to provide fluid flow in the channels via introduction and removal of fluid via inlet to and outlet from the channel(s). Rather, the volume of fluid contained within the channel itself may provide sufficient support of biological material in the respective channels. In contrast, the embodiment of FIGS. 1A-1C may be referred to as a “dynamic configuration” because flow rates in the channels may be controllably varied, including via pumps and the like. The acoustic exit window may be a thin membrane (FIG. 45, left panel), or may have an added acoustic absorption material (FIG. 45, right panel). The electrode connections of the basolateral chamber/channel may have an alternative connection, so long as the electrodes are in electrical contact with the medium in the chamber/channel. A focused acoustic wave 102′ may be provided at the porous membrane location, where cells 140′ are located.

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect 13) that references an aspect (e.g., Aspect 1) for which there are sub-aspects having the same top level number (e.g., Aspect 1A, 1B, 1C, and so forth) necessarily includes reference to those sub-aspects 1A, 1B, 1C, and so forth. Furthermore, it is explicitly contemplated that aspects can be combined in any manner. Moreover, the term “any preceding aspect” means any aspect that appears prior to the aspect that contains such phrase (in other words, the sentence “Aspect 100: The method of any one of aspects 50-99, or any preceding aspect, . . . ” means that any aspect prior to aspect 100 is referenced, including aspects 1-49). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even though “less than Y” in isolation does not include Y.

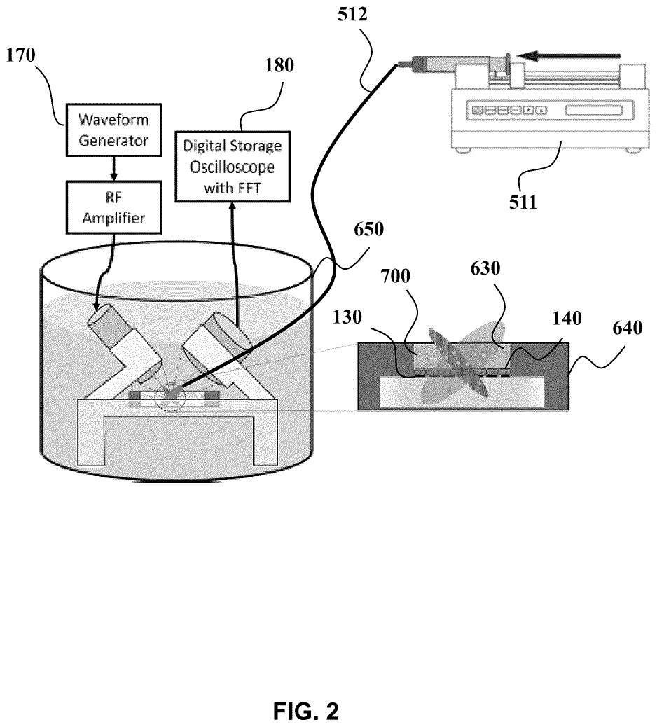

Aspect 1. An apparatus 100 (see FIG. 1A) for manipulating a cellular interaction with an acoustic wave 102, the apparatus comprising:
a bottom layer 110 comprising:
   a first fluidic channel 112;
a top layer 120 comprising:
   a second fluidic channel 122;
   (optionally) a second fluidic inlet 124 fluidically connected to the second fluidic channel 122 and configured to introduce a second fluid to the second fluidic channel 122; and
   (optionally) a second fluidic outlet 126 fluidically connected to the second fluidic channel 122 and configured for removal of the second fluid from the second fluidic channel 122;
an intermediate membrane 130 positioned between the top layer 120 and the bottom layer 110, the intermediate layer 130 comprising a receiving surface 132, wherein the intermediate layer 130 is in fluidic contact with the first fluidic channel 112 and the second fluidic channel 122;
a biological material 140 positioned inside the first 112 and/or second fluidic channels 122 and/or on the receiving surface 132 of the intermediate membrane 130;
(optionally) a first fluidic inlet 150 passing through the top layer 120 and the intermediate membrane 130, fluidically connected to the first fluidic channel 112, and configured to introduce a first fluid to the first fluidic channel 112; and
(optionally) a first fluidic outlet 160 passing through the top layer 120 and the intermediate membrane 130, fluidically connected to the first fluidic channel 112 and configured for removal of the first fluid from the first fluidic channel 112;
wherein the bottom layer 110, top layer 120 and intermediate membrane 130 are independently configured for acoustic transparency;
a mechanical (e.g., an acoustic) wave generator 170 (see FIG. 2) configured to provide the mechanical (e.g., acoustic) wave 102 to the biological material 140;
a biomarker parameter monitor 180 configured to monitor a change in one or more biomarkers after provision of the mechanical (e.g., acoustic) wave 102.
The optional aspects are to provide dynamic control of fluid flow in the first and second fluidic channels. As desired, a bottom compartment 113 (basolateral compartment) may contain a hydrogel. For example, basolateral compartment of the apparatus filled with hydrogel facilitates permeability characterization. Additionally, vessel structures can be included within the hydrogel. The hydrogel compartment may be provided in the bottom layer, and may have a means for introducing hydrogel to the hydrogel compartment, such as via a hydrogel passage(s), syringe-type interface, and the like, for introducing and/or removal of hydrogel, including to first or the second fluidic channel. Optionally, a transendothelial electrical resistance (TEER) monitor 511 comprising a TEER cable 512 is electrically connected to the biological material, including via on of the fluidic channels.

Aspect 2. The apparatus 100 of Aspect 1, wherein the biological material comprises an isolated biological tissue or cultured cells.

Aspect 3. The apparatus 100 of Aspect 1 or 2, wherein the biological material comprises one or more monolayers of cells.

Aspect 4. The apparatus 100 of any one of the preceding Aspects, wherein the biological material comprises a blood-brain interface or a model of a blood-brain interface.

Aspect 5. The apparatus 100 of any one of the preceding Aspects, wherein the biological material comprises in vitro cultured mammalian cells related to animal and human tissue barriers, such as selected from the group consisting of iPS-derived cells, immortalized human primary cells, Caco-2 cells, MDCK cells, Endo-1 cells, and HBEC-5i cells, including in vitro cultured mammalian cells selected from the group consisting of iPS-derived cells, Caco-2 cells, Endo-1 cells, and HBEC-5i cells.

Aspect 6. The apparatus 100 of any one of the preceding Aspects, wherein the biological material is part of a blood-brain-barrier model.

Aspect 7. The apparatus 100 of any one of the preceding Aspects, wherein a portion of the second fluidic channel is positioned above a portion of the first fluidic channel to form a combined channel volume from an overlapping portion of the portion of the second fluidic channel with the portion of the first fluidic channel.

Aspect 8. The apparatus 100 of Aspect 7, wherein the first fluidic channel 112 is orthogonal to the second fluidic channel 122.

Aspect 9. The apparatus 100 of any one of the preceding Aspects, wherein:
the first fluidic channel 112 has a non-uniform width with a maximum width positioned at a midpoint between the first fluidic inlet and the first fluidic outlet; and
the second fluidic channel 122 has a non-uniform width with a maximum width positioned at a midpoint between the second fluidic inlet and the second fluidic outlet.

Aspect 10. The apparatus 100 of any one of the preceding Aspects, wherein the bottom layer and/or top layer comprises polydimethylsiloxane (PDMS).

Aspect 11. The apparatus 100 of any one of the preceding Aspects, wherein the bottom layer 110 and/or top layer 120 comprises a thickness of between 0.001 mm and 10 mm.

Aspect 12. The apparatus 100 of any one of the preceding Aspect,
wherein the bottom layer 110 further comprises:
   a first piece of tubing 510 fluidically connected to the first fluidic inlet 150, and
   a second piece of tubing 520 fluidically connected to the first fluidic outlet 160;
wherein the top layer 120 further comprises:
   a third piece of tubing 530 fluidically connected to the second fluidic inlet 124, and
   a fourth piece of tubing 540 fluidically connected to the second fluidic outlet 126;
wherein each of the first 510, second 520, third 530, and fourth 540 pieces of tubing are configured to be filled with a culture media 545 that supports viability of the biological material 140.

Aspect 13. The apparatus 100 of any one of the preceding Aspects, the first fluidic channel 112 further comprising a first electrode 550 and a second electrode 560, and the second fluidic channel 122 further comprising a third electrode 570 and a fourth electrode 580. (see FIG. 1A)

Aspect 14. The apparatus 100 of Aspect 13 or any preceding Aspect, wherein each of the first and second electrode 550, 560 are independently microfabricated on an inner surface 118 of the first fluidic channel 112, and each of the third and fourth electrodes 570, 580 are independently microfabricated on an inner surface 128 of the second fluidic channel 122.

Aspect 15. The apparatus 100 of any one of the preceding Aspects, wherein the intermediate membrane 130 is porous.

Aspect 16. The apparatus 100 of any one of the preceding Aspects, wherein the intermediate membrane 130 comprises a material selected from the group consisting of polyester polytetrafluoroethylene (PETE), polydimethylsiloxane (PDMS), polycarbonate, and parylene.

Aspect 17. The apparatus 100 of any one of the preceding Aspects, wherein the intermediate membrane 130 comprises a thickness of between 0.01 μm and 20 μm.

Aspect 18. The apparatus 100 of any one of Aspects 15-17 or any preceding Aspect, wherein the membrane 130 comprises an average pore size of between 0.01 μm and 20 μm.

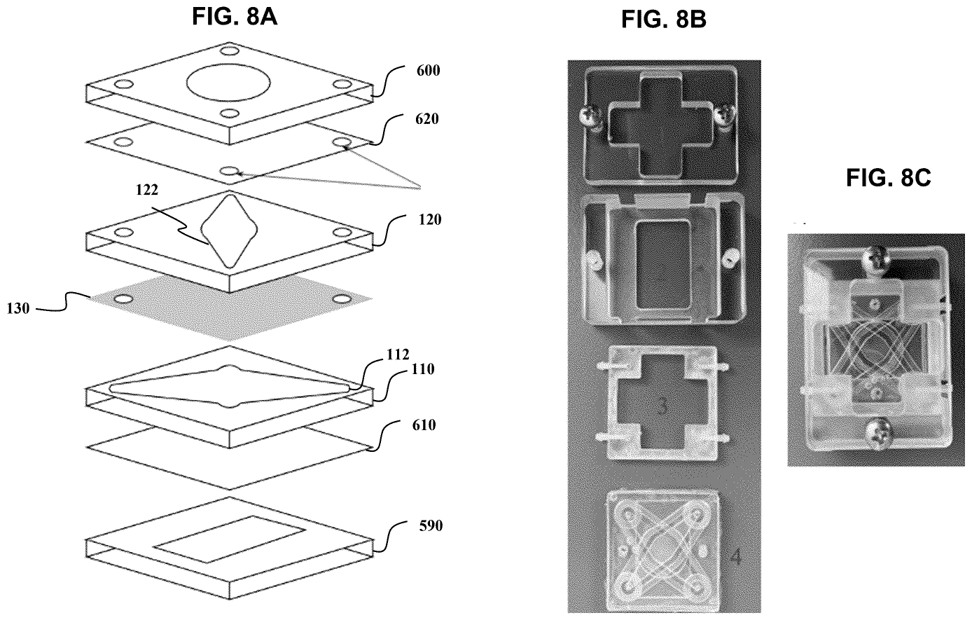

Aspect 19. The apparatus 100 of any one of the preceding Aspects, wherein:
the bottom layer 110 comprises a bottom viewing frame 590 (see FIG. 8A) positioned below the first fluidic channel 112, the bottom viewing frame 590 comprising the first fluidic inlet 150 and the first fluidic outlet 160; and
the top layer 120 comprises a top viewing frame 600 positioned above the second fluidic channel 122, the top viewing frame 600 comprising the second fluidic inlet 124 and the second fluidic outlet 126.

Aspect 20. The apparatus 100 of Aspect 19, wherein the bottom viewing frame 590 and/or top viewing frame 600 comprises a material selected from the group consisting of: polydimethylsiloxane (PDMS), polycarbonate, poly(methyl methacrylate) (PMMA), and glass.

Aspect 21. The apparatus 100 of Aspect 19 or 20, or any preceding Aspect, wherein the bottom viewing frame 590 and/or top viewing frame 600 comprises a thickness of between 0.1 mm and 10 mm.

Aspect 22. The apparatus 100 of any one of Aspects 19-21 or any preceding Aspect, wherein:
the bottom layer 110 further comprises a first thin layer 610 positioned between the first fluidic channel 112 and the bottom viewing frame 600, thereby preventing fluid from exiting the first fluidic channel 112 other than at the first fluidic outlet 160 and preventing fluid from entering the first fluidic channel 112 other than at the first fluidic inlet 150; and
the top layer 120 further comprises a second thin layer 620 positioned between the second fluidic channel 122 and the top viewing frame 600, thereby preventing fluid from exiting the second fluidic channel 122 other than at the second fluidic outlet 126 and preventing fluid from entering the second fluidic channel 122 other than at the second fluidic inlet 124.

Aspect 23. The apparatus 100 of Aspect 22 or any preceding Aspect, wherein each of the thin layers 610, 620 comprises a material selected from the group consisting of polydimethylsiloxane (PDMS), parylene, poly-oxydiphenylene-pyromellitimide (Kapton), biaxially oriented polyethylene terephthalate (boPET; Mylar), and glass.

Aspect 24. The apparatus 100 of Aspect 22 or 23 or any preceding Aspect, wherein each of the thin layers 610, 620 have a thickness of between 0.01 μm and 100 μm.

Aspect 25. The apparatus 100 of any one of the preceding Aspects, wherein the acoustic wave generator 170 is configured to introduce an acoustic waveform 102 to the biological material 140.

Aspect 26. The apparatus 100 of Aspect 25, wherein the acoustic wave generator 170 is further configured to:
focus the acoustic waveform 102 on the top layer 120; and
tilt from a position normal to the top layer 120 to reduce acoustic waves reflected from the top layer 120 back to the acoustic wave generator 102.

Aspect 27. The apparatus 100 of Aspect 25 or 26 or any preceding Aspect, the apparatus 100 further comprising gas bubbles 630 within a fluid 640, wherein the gas bubbles 630 are configured to cavitate upon interaction with the acoustic waveform 102 and thereby interact with the biological material 140.

Aspect 28. The apparatus 100 of any one of Aspects 25-27 or any preceding Aspect, wherein the acoustic waveform 102 comprises acoustic pulses with an acoustic frequency selected from the range of 100 kHz to 20 MHz, wherein each pulse comprises a pulse duration selected from the range of 100 ns to 2 s.

Aspect 29. The apparatus 100 of any one of Aspects 25-27 or any preceding Aspect, wherein the acoustic waveform 102 comprises a continuous acoustic wave with an acoustic frequency selected from the range of 100 kHz to 20 MHz, or an ultrasound waveform with the acoustic wave generator 170 that is an ultrasound generator.

Aspect 30. The apparatus 100 of any one of the preceding Aspects, wherein the biomarker parameter comprises a parameter that can be affected by acoustic waves.

Aspect 31. The apparatus 100 of any one of the preceding Aspects, wherein the biomarker parameter comprises a measure of cellular barrier disruption, a protein, mRNA, and/or a polynucleotide.

Aspect 32. The apparatus 100 of Aspect 31 or any preceding Aspect, wherein the biomarker parameter comprises a measure of cellular barrier disruption selected from the group consisting of: a permeability parameter of the biological material, a temperature parameter of the biological material, and a transendothelial electrical resistance (TEER) parameter of the biological material.

Aspect 33. The apparatus 100 of any one of the preceding Aspects, the apparatus further comprising a container 650 (see FIG. 2) configured to hold a fluid 640, wherein at least a portion of a surface(s) of said container 650 is covered with an ultrasound absorbing material 660.

Aspect 34. The apparatus 100 of Aspect 33 or any preceding Aspect, wherein the container 650 is filled with water, and wherein the bottom layer 110, the top layer 120, the intermediate membrane 130, and the biological material 140 are submerged in said water.

Aspect 35. The apparatus 100 of Aspect 34 or any preceding Aspect, wherein the acoustic wave generator 170 is submerged in said water.

Aspect 36. The apparatus 100 of any one of Aspects 33-35 or any preceding Aspect, wherein an air/water interface is covered with an ultrasound absorbing material 660.

Aspect 37. The apparatus 100 of any one of Aspects 33-36 or any preceding Aspect, wherein the ultrasound absorbing material 660 is configured to reduce reflections of the acoustic waveform 102 that is an ultrasonic waveform.

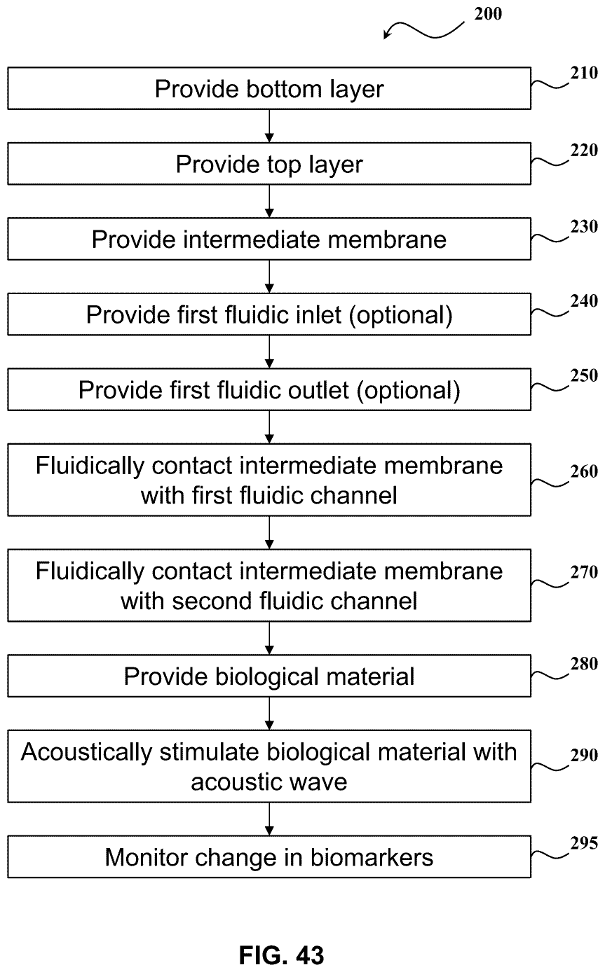

Aspect 38. A method 200 (see FIG. 43) of making an apparatus 100 for the study of cellular interaction with ultrasound, the method comprising:
(210) providing a bottom layer 110 comprising:
   a first fluidic channel 112 configured to be in fluidic contact with an intermediate membrane 130;
   wherein the bottom layer 110 is configured for acoustic transparency;
(220) providing a top layer 120 comprising:
   a second fluidic channel 122 configured to be in fluidic contact with said intermediate membrane 130;
   a second fluidic inlet 124, allowing the introduction of a fluid into the second fluidic channel 122;
   a second fluidic outlet 126, allowing the flow of a fluid out of the second fluidic channel 122; and
   wherein the top layer 120 is configured for acoustic transparency
(230) providing the intermediate membrane 130 comprising a receiving surface 132, wherein the intermediate membrane 130 is configured for acoustic transparency;
(240) (optional for dynamic fluid control and/or gas exchange, such as CO₂ removal from fluidic channel(s)) providing a first fluidic inlet 150 passing through the top layer 120 to reach the bottom layer 110, allowing the introduction of a fluid into the first fluidic channel 112;
(250) (optional—see above) providing a first fluidic outlet 160 passing through the top layer 120 to reach the bottom layer 110, allowing the flow of a fluid out of the first fluidic channel 112;
(260) fluidically contacting the intermediate membrane 130 with the first fluidic channel 112;
(270) fluidically contacting the intermediate membrane 130 with the second fluidic channel 122;
(280) providing a biological material 140 to the first fluidic channel 112 and/or the second fluidic channel 122 and/or the receiving surface 132 of the intermediate membrane 130;
(290) acoustically stimulating the biological material 140 with an acoustic wave 102 using an acoustic wave generator 170; and
(295) monitoring a change in one or more biomarkers after acoustic stimulation using a biomarker parameter monitor 180.

Aspect 39. The method 200 of Aspect 38 or any preceding Aspect, wherein the biological material 140 comprises an isolated biological tissue or cultured cells.

Aspect 40. The method 200 of Aspect 38 or 39 or any preceding Aspect, wherein the biological material 140 comprises one or more monolayers of cells.

Aspect 41. The method 200 of any one of Aspects 38-40 or any preceding Aspect, wherein the biological material 140 comprises a blood-brain interface or a model of a blood-brain interface.

Aspect 42. The method 200 of any one of Aspects 38-41 or any preceding Aspect, wherein the biological material 140 comprises in vitro cultured mammalian cells, including in vitro cultured mammalian cells selected from the group consisting of iPS-derived cells, Caco-2 cells, Endo-1 cells, and HBEC-5i cells.

Aspect 43. The method 200 of any one of Aspects 38-42 or any preceding Aspect, wherein providing the bottom layer 110 and/or top layer 120 comprises injection molding.

Aspect 44. The method 200 of Aspect 43 or any preceding Aspect, wherein said injection molding comprises soft lithography.

Aspect 45. The method 200 of any one of Aspects 38-44 or any preceding Aspect, wherein providing the intermediate membrane 130 comprises:
providing a thin layer of material; and
laser cutting the intermediate membrane 130 from the thin layer of material.

Aspect 46. The method 200 of any one of Aspects 38-45 or any preceding Aspect, wherein the bottom and top layers 110, 120 each comprise polydimethylsiloxane (PDMS), and the intermediate membrane 130 comprises a material selected from the group consisting of polyester polytetrafluoroethylene (PETE), polydimethylsiloxane (PDMS), polycarbonate, and parylene.

Aspect 47. The method of any one of Aspects 38-46, the method 300, further comprising:
(310) attaching a first thin layer 610 to the first fluidic channel 112;
(320) attaching a bottom viewing frame 590 to the first thin layer 610;
(330) attaching a second thin layer 620 to the second fluidic channel 122;
(340) attaching a top viewing frame 600 to the second thin layer 620.

Aspect 48. The method 300 of Aspect 47 or any preceding Aspect, wherein:
the bottom layer 110 comprises a bottom viewing frame 590;
the top layer 120 comprises a top viewing frame 600; and
the bottom viewing frame 590 and/or top viewing frame 600 are formed by injection molding.

Aspect 49. The method 300 of Aspect 48 or any preceding Aspect, wherein said injection molding comprises soft lithography.

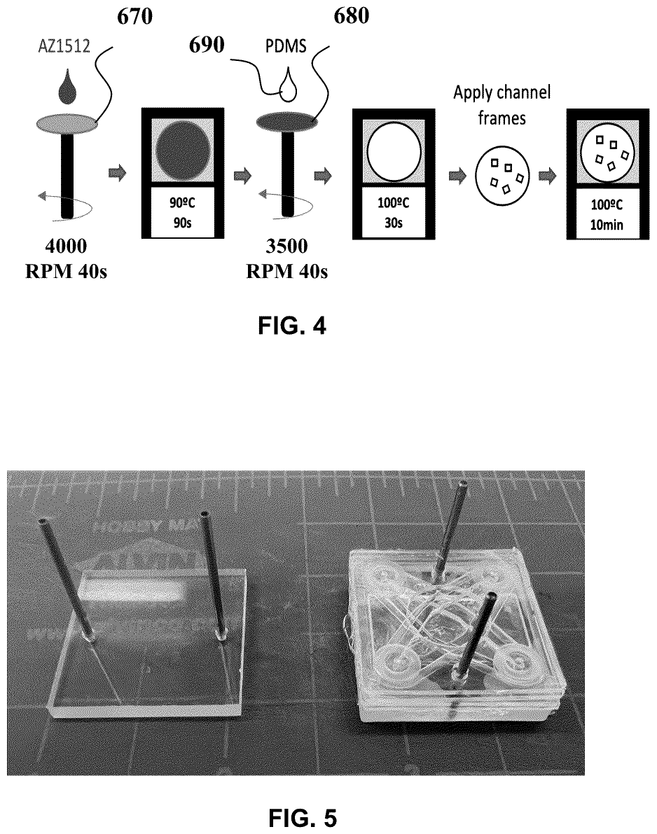

Aspect 50. The method 300 of any one of Aspects 47-49 or any preceding Aspect, wherein each of the first and second thin layers 590, 600 are formed by:
providing a silicon wafer 670 (see FIG. 4);
cleaning the silicon wafer 670;
applying a photoresist 680 to the silicon wafer 670;
curing the photoresist 680 to the silicon wafer 670;
applying a material 690 to the cured photoresist 680; and
curing the material 690.

Aspect 51. The method 300 of Aspect 50 or any preceding Aspect, wherein:
applying the photoresist 680 to the silicon wafer 670 comprises spin coating; and
applying the material 690 to the cured photoresist 680 comprises spin coating.

Aspect 52. The method 300 of any one of Aspects 48-51 or any preceding Aspect, wherein:
the bottom viewing frame 590 and the top viewing frame 600 each independently comprise a material selected from the group consisting of polydimethylsiloxane (PDMS), polycarbonate, poly(methyl methacrylate) (PMMA), and glass; and
the first thin layer 610 and the second thin layer 620 each independently comprise a material selected from the group consisting of polydimethylsiloxane (PDMS), parylene, poly-oxydiphenylene-pyromellitimide (Kapton), biaxially oriented polyethylene terephthalate (boPET; Mylar), and glass.

Aspect 53. A method 400 (see FIG. 45) of evaluating a cellular interaction with an acoustic wave, the method comprising:
(410) providing the apparatus 100 of any one of Aspects 1-37;
(420) introducing the first fluid into the first fluidic channel 112;
(430) introducing the second fluid into the second fluidic channel 122;
(440) wherein the introduced first fluid and second fluid are provided to the biological material 140;
(450) applying an acoustic waveform 102 to the biological material 140; and
(460) monitoring a biomarker parameter of the biological material 140, thereby evaluating the cellular interaction with ultrasound.

Aspect 54. The method 400 of Aspect 53 or any preceding Aspect, wherein applying an acoustic wave 102 to the biological material 140 comprises:
applying acoustic pulses to the biological material 140 with an acoustic frequency selected from the range of 100 kHz to 10 MHz, wherein each pulse comprises a pulse duration selected from the range of 100 ns to 2 s.

Aspect 55. The method 400 of Aspect 53 or any preceding Aspect, wherein applying ultrasound to the biological material 140 comprises:
applying a continuous acoustic wave 102′ to the biological material 140 with an acoustic frequency selected from the range of 100 kHz to 10 MHz.

Aspect 56. The method 400 of any one of Aspects 53-55 or any preceding Aspect, wherein monitoring the biomarker parameter of the biological material 140 comprises:
monitoring the biomarker parameter of the biological material 140 with respect to a molecular tracer 700 (see FIG. 2).

Aspect 57. The method 400 of Aspect 56 or any preceding Aspect, wherein the molecular tracer 700 comprises 70 k-Dextran-TMR or Lucifer Yellow.

Aspect 58. The method 400 of any one of Aspects 53-57 or any preceding Aspect, further comprising the step of co-culturing at least two different biological cells.

Aspect 59. The method 400 of any one of Aspects 53-58 or any preceding Aspect, wherein the monitoring step comprises measuring an electrical parameter of the biological material with a plurality of electrodes (e.g., 550, 560, 570, and/or 580).

### EXAMPLES

### Example 1: Development of an Ultrasound-Transparent Organ-On-Chip Platform Towards Modeling

The blood-brain-barrier (BBB) is an obstacle for treating glioblastomas and other neurological disorders. Bubble-assisted focused ultrasound (BAFUS) medicated BBB disruption is a promising technology that enables the delivery of large drug doses at targeted locations across the BBB. However, the current lack of an in vitro model of this process hinders the full understanding of BAFUS BBB disruption for better translation into clinics. In an embodiment, a US-transparent organ-on-chip device is provided that can be useful for the in vitro modeling of the BAFUS BBB disruption. The transparency of the device window to focused ultrasound (FUS) is calculated theoretically and demonstrated by experiments. The fluidic flow and drug diffusion within the device are modeled using finite element methods. Nanobubbles are fabricated, characterized by cryogenic transmission electron microscopy (cryo-TEM), and show bubble cavitation under FUS. Human colorectal adenocarcinoma (Caco-2) cells are used to form a good cellular barrier for BAFUS barrier disruption, as suggested by the permeability and transepithelial electrical resistance (TEER) measurements. Finally, barrier disruption and recovery are observed in BAFUS disrupted US-transparent organ-on-chips with Caco-2 barriers.

When micro/nanobubbles injected into the blood are stimulated by a FUS beam at low frequency (~1 MHz or lower for low scattering by the skull) near a glioblastoma, they cavitate to generate acoustic pressure and physically disrupt the BBB to allow chemotherapeutic drugs to move into the tumor site (4). The disruption can be temporary with BBB recovery observable 4-6 hours after BAFUS (5). It provides a way to deliver large dose of drugs across BBB at targeted locations.

In an embodiment, a design for an organ-on-chip platform can be used to test how BBB disruption responds to the different BAFUS parameters. In an embodiment, a BBB-on-Chip is transparent to ultrasound, enabling accurate BAFUS power administration that has not been reported in the literature. Another advantage of this technology is its optical transparency that makes the BBB-on-Chip easy to image and monitor throughout culture.

Referring to FIG. 2, an exemplary setup for is shown. In an embodiment, a 1 MHz FUS signal is applied to a US-transparent chip with a cellular barrier. The process is monitored by the subharmonic peak (at 0.5 MHz) of the bubble cavitation using Fast Fourier Transform (FTT) in the frequency domain.

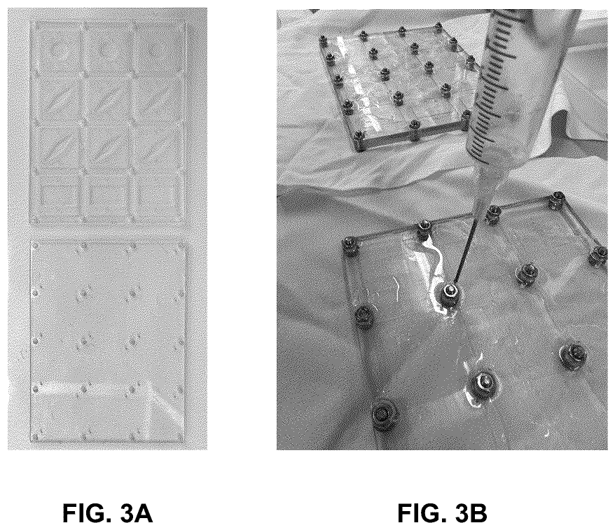

US-transparent Organ-on-Chip Device Fabrication: Injection Molding: In an embodiment, four PDMS frames that comprise the US-transparent chip may be created using injection molding. The mold, as seen in FIG. 3A, comprises two pieces: a bottom piece with frame reservoirs and a top cover containing injection inlet and vent outlet holes for each frame. The two pieces are held together securely by nuts and bolts. The top of FIG. 3B shows a mold with nuts and bolts in place, ready for injection. Each mold contains enough frames for 3 complete devices.

In an embodiment, before injection, a ratio of 10:1 PDMS (base to crosslinker by mass) is thoroughly mixed and then placed in a vacuum degasser for at least 30 minutes or until no bubbles are visible. The uncured PDMS is then carefully transferred to a 10 ml syringe with a 25-gauge blunt needle. As shown in the bottom of FIG. 3B, the needle is then placed in the inlet hole of each frame and the syringe is pressed forcefully until the entire frame is filled. If bubbles emerge, more PDMS from the syringe is used to push them out through the pressure outlet hole. Each frame reservoir is filled individually while taking care not to exert too much pressure on the whole mold as that could cause more bubble formation. After injection is complete, the molds are allowed to cure for 48 hours at room temperature (RT), or 24 hours followed by a 1-hour oven treatment at 60° C. Once cured, the PDMS frames are carefully removed from the mold and excess PDMS is carefully removed with a sharp blade.

PDMS Membrane Fabrication: In an embodiment, a second stage of the US-transparent chip fabrication is to create and bond thin PDMS membranes to the top and bottom channel frames previously molded. As outlined in FIG. 4, a silicon wafer is used as a substrate for the process. The wafer is thoroughly cleaned with acetone, then isopropanol, and then methanol before processing. AZ1512 photoresist is used to coat the wafer as a non-adherent coating to allow for the easy removal of fully cured PDMS. The AZ1512 is spun at 4000 RPM and then cured at 90° C. for 90 s to achieve a thickness of 1.2 μm (15). PDMS is coated on top of the cured photoresist by spinning at 3500 RPM to produce the thin membrane thickness of 20 μm (16). The membrane thickness is also confirmed using a Dektak V200-SI stylus profilometer. The PDMS coating is then partially cured at 100° C. for 30 sec.

In an embodiment, to bond the thin PDMS membrane to the middle frames, the middle frames are cleaned with tape first to remove particles on their surfaces and then placed on top of the PDMS thin membrane immediately after the 30 s partial cure. The wafer is then returned to the hot plate and left to continue curing for 10 min at 100° C. To ensure the membranes are completely cured, they are allowed to sit at RT overnight, followed by an hour of oven treatment at 60° C.

Device Assembly: In some embodiments, the final stage of device fabrication is the assembly of individual components. Uncured PDMS is used as a glue to bond the frames and membranes. The uncured PDMS glue is prepared on a Si wafer by spin coating at a slow 500 RPM for 1 min to achieve a 0.5 mm thickness. The surfaces of frames to be bonded are laid onto the wafer to apply the PDMS glue. The frames are then stacked on top of each other in the right order through two guiding holes using two blunt 15-gauge needles, as shown in FIG. 5. The PETE membrane is cut to the same size as the frame (28×28 mm) with guiding holes and inlet/outlet holes using a VersaLaser VLS3.50 laser cutter. After stacking the frame and membrane layers, the device is cured at RT for 24 hours followed by a 1-hour oven treatment at 60° C.

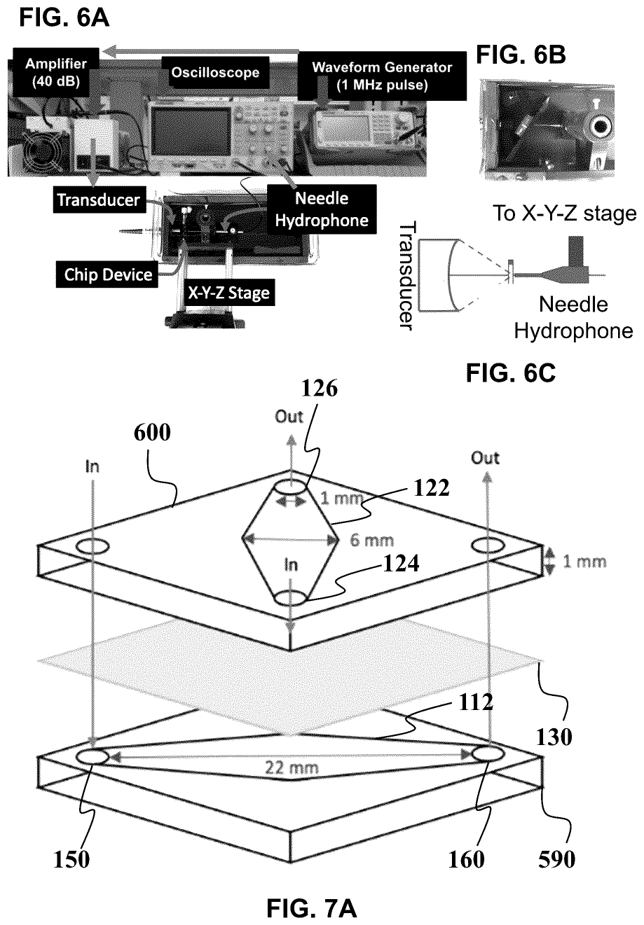

Device Characterization: US Transparency Characterization: FIG. 6A shows an exemplary setup for device US transparency characterization. In an embodiment, a waveform generator (Siglent SDG 1032X) is used to generate 1 MHz burst signal (10 ms, 10 k cycles) with repetition rate of 1 Hz. The signal is amplified by a 43 dB 20 W RF amplifier (NP961 from NP Technologies), then sent to a 1 MHz focused Olympus A303S US transducer (15 mm focal length, transverse and axial beam sizes of 1.9 mm and 14 mm). The transducer is mounted on the sidewall of a water tank with the US emission along the long axis of the tank horizontally. During US measurement, the tank is filled with degassed, deionized (DI) water with the transducer submerged. The water degassing is done by placing the container in a desiccator with −90 kPa vacuum for 30 minutes while stirring with a 2-inch magnetic stirrer at 400 RPM. The inner surfaces of the tank are covered by an US absorbing pad (blue material in FIG. 5).

A 1 mm needle hydrophone (Precision Acoustics, UK) is used to measure the intensity of the FUS beam. The hydrophone is mounted to a 3D printed holder and a custom XYZ stage is used to change the position of the hydrophone. The measurement is done under the degassed DI water. The holder also has a slot to insert the organ-on-chip device with the US-transparent window of the device between the transducer and the hydrophone to measure any US energy loss due to the membrane window.

To mimic the tilted incident angle of FUS beam in a barrier disruption experiment, the US transparency characterization is repeated with the device tilted 31° to the transduce-hydrophone axis, as shown in FIG. 6B.

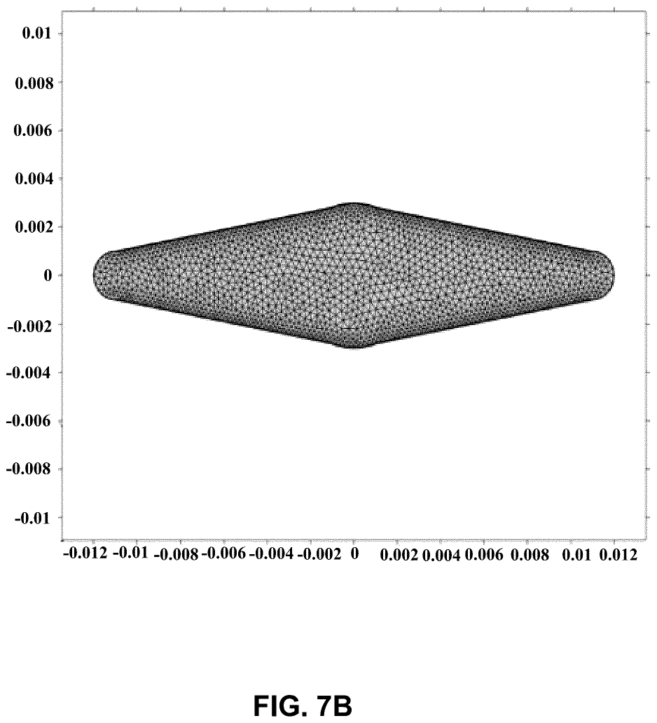

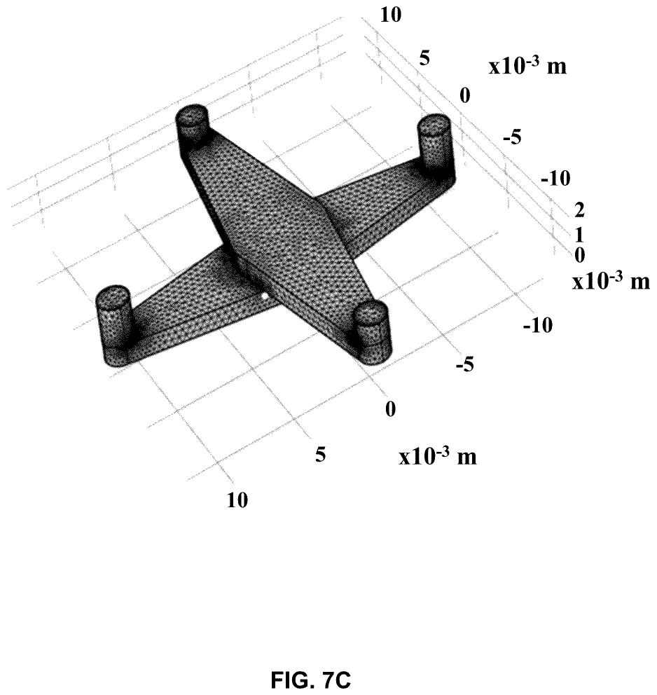

Finite Element Modeling: In an embodiment, finite element modeling is used to predict fluid velocity and pressure profiles as well as diffusion gradients in the organ-on-Chip device. The dimensions of the top and bottom channels are provided in FIG. 7A. The resulting geometry and mesh generated in COMSOL is provided in FIGS. 7B and 7C. A 2D mesh and a 3D mesh are created for the laminar flow model, while only a 3D mesh is created for the transport of diluted species model. Both meshes are physics-controlled meshes of quadratic triangular elements auto-generated by the software.

Nanobubble Fabrication and Characterization: Nanobubble Formulation: In an embodiment, five different lipids are purchased from Avanti Polar Lipids (Birmingham, AL) to fabricate the nanobubbles: 1,2-Dipalmitoyl-sn-glycero-3-phosphate (DPPA), 1,2-dipalmitoyl-sn-glycero-3-phosphocholine (DPPC), 1,2-Dipalmitoyl-sn-glycero-3-phosphoethanolamine (DPPE), 1,2-dibehenoyl-sn-glycero-3-phosphocholine (DBPC), and 1,2-Distearoyl-sn-Glycero-3-Phosphoethanolamine with conjugated methoxyl poly(ethylene glycol) (DSPE-mPEG). The nanobubble fabrication protocol is adapted from previous studies (22, 23) as follows. A 6:1:2:1 ratio of DBPC (or DPPC):DPPA:DPPE:DSPE-mPEG is combined and dissolved in chloroform solution. The chloroform is warmed to 80° C. and allowed to evaporate for 3 hours. The lipid powder residue is hydrated in a PBS solution containing 5% glycerol and 0.6% Pluronic L10 at a concentration of 10 mg/ml by stirring at 300 RPM on a 125° C. hot plate for 3 hours. The solution is then aliquoted into smaller rubber septum sealed vials and the air in the vials is replaced with octafluoropropane (C3F8). At this point the bubble vials are either stored or activated. To activate the bubbles, the vial is violently shaken in an amalgamator at maximum speed for 45 sec. After amalgamation, a clear divide between white foam and darker opaque solution appear when the vial is inverted. Care is taken to only collect the solution that resides below the interface in the darker solution region. This solution is then diluted either 100× or 500× for use in characterization and BAFUS experiments.

BAFUS Setup and Nanobubble Excitation: In an embodiment, the BAFUS setup is similar to the US setup used for transparency characterization shown in FIG. 6A. However, the needle hydrophone is replaced with a 0.5 MHz Olympus focused receiving transducer. Both the emitting (1 MHz) and receiving (0.5 MHz) transducers are placed in the angled holder shown in FIG. 2. A 1 L cylindrical glass beaker layered with US absorbing pads is used to house the setup and degassed DI water. For all BAFUS experiments, the waveform generator is set to a 1 MHz pulse (10,000 cycles and a 1 sec burst period), 100 m Vrms, and a 50-ohm load. The oscilloscope is set to read FFT data at a range of 0-10 MHz.

Nanobubble Characterization Using FUS: In an embodiment, the bubbles are first diluted to either 100× or 500× in PBS after activation and then injected into the top channel of the device using a syringe pump. Standard 1×PBS is loaded into the bottom channel. Once the device is loaded with PBS and nanobubble solution, the syringe pump is stopped and FUS is delivered while the solutions are static. To characterize the nanobubbles, enhancement (dB) peak height of the 0.5 MHz signal is recorded every second until the peak disappears using a handheld video camera. Two recipes are characterized: one using DPPC as the primary lipid and the other using DBPC as the primary lipid. The peak height in dB is plotted vs time to produce a peak enhancement curve. The peak of this curve is named signal enhancement. The half-life is measured from time 0 to the time when enhancement decreased by 6 dB from the peak. A decrease of 6 dB from the peak is considered a decrease in half the maximum power generated by bubble stable cavitation. A longevity of nanobubbles is also studied for the 100× DBPC recipe for a constant flow condition with a nanobubble flow rate of 15 ml/hr. The 0.5 MHz peak height is plotted during the constant flow until the peak disappears.

Transmission Electron Microscopy (TEM) Characterization: In an embodiment, the 100×DBPC bubbles are imaged under cryogenic transmission electron microscopy (Cryo-TEM). The general process for preparing cryogenic transmission electron microscopy samples has been previously described in literature (24). In summary, DBPC recipe samples are placed on a gold microgrid sample and wicked for 6 sec before instantly freezing them to create a vitreous ice on the grid. The duration of wicking determines the thickness of the ice. This vitreous ice is then placed in the transmission electron microscope for imaging. Individual images of the nanobubbles are visualized in the open squares of the sample grid.

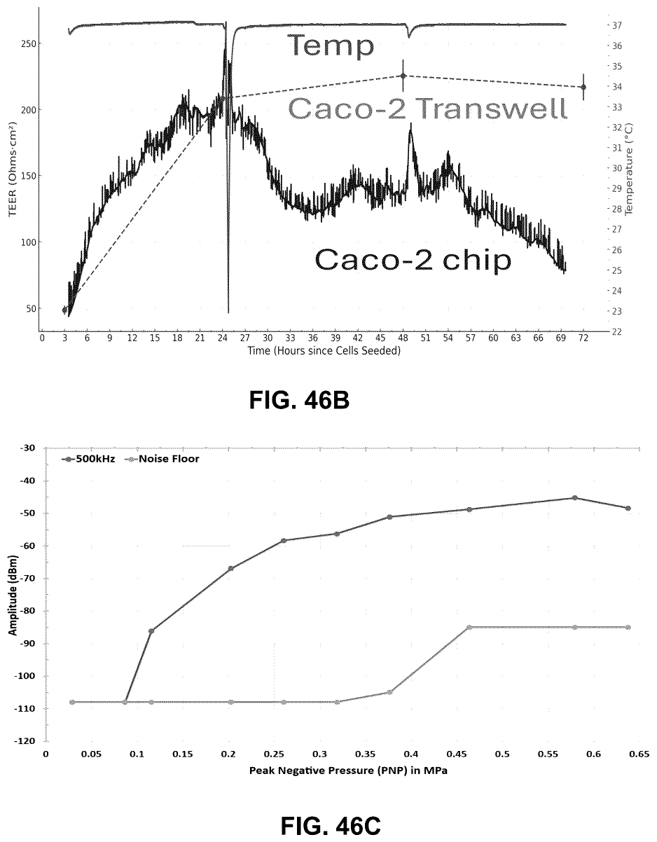

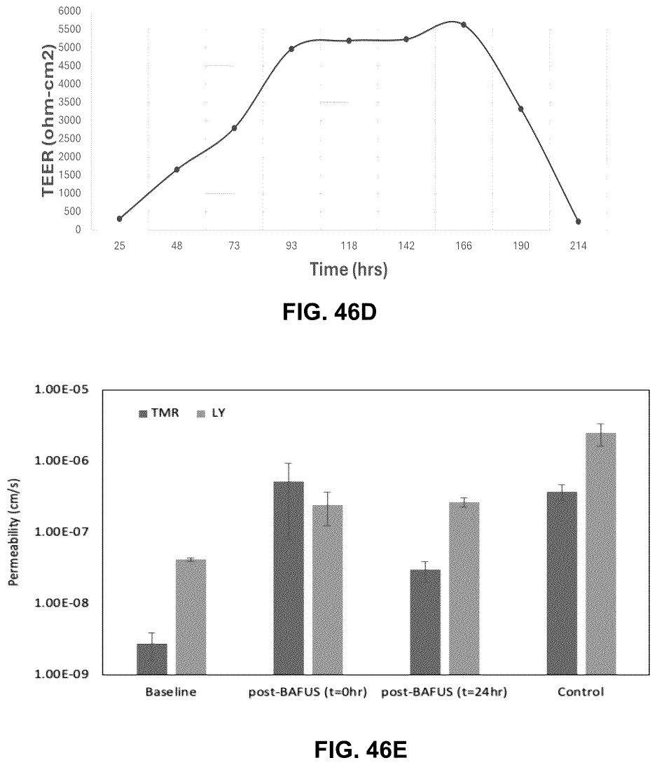

Cell Culture Optimization in Transwells: Transwell Culture: In an embodiment, a panel of cell types is cultured in transwells to determine the best route to take for BAFUS treatment: in-house differentiated brain endothelial cells from iPSCs, Human Brian Endothelial Cells cell line (HBEC-5i) purchased from ATCC, and Caco-2 cells also purchased from ATCC. Selected iPSC lines are derived from non-diseased subjects with an age range 65-69, high diversity of progeny, and comprehensive molecular data (26). These iPSCs are differentiated into brain endothelial cells (Endo-1) according to a previous study that detailed a specific differentiation protocol (27). Transwell inserts are coated with 1% gelatin for at least 3 hours at 37° C. before seeding any cells. During preliminary panel experiments where multiple cell types are cultured on a single plate, Caco-2 cells are cultured without any prior coating. Transwells are seeded with 33 k cells of each cell type with three replicates. Two control transwells are filled with media, one coated and one uncoated. After Caco-2 cells are selected, their culture is optimized by testing a 10% collagen coating for 3 hours at 37° C. and higher seeding densities of 150 k and 200 k cells per insert. To culture the endothelial cells, the top and bottom wells are filled with 200 μl and 500 μl of endothelial cell growth medium (ATCC). Caco-2 cells are cultured with DMEM, 20% FBS, 4.5 g/l glucose, 25 mM HEPES medium. Cell media is changed every 24 hours or when measurements were taken. FIG. 46 illustrates some transwell configurations.

TEER: In an embodiment, STX2 chopstick electrodes and an EVOM2 epithelial voltammeter from World Precision Instruments are used to perform TEER measurements. The electrodes are calibrated with known KCl concentrations according to manufacturer's specifications. The voltammeter is calibrated using a 5000-ohm resistor. To measure TEER, a custom plate cover is machined with holes at each well to allow for electrode insertion while holding all the inserts securely in place. The short end of the chopstick electrode is inserted into the top compartment and the longer end is inserted into the bottom compartment to measure the total resistance of the membrane or monolayer. This measured value is multiplied by the area of the membrane in the transwell (0.33 cm2) to determine the TEER value. TEER values of cell monolayers are further adjusted against controls by subtracting an average background value of the membrane controls.

Permeability: In an embodiment, permeability measurements are conducted in addition to TEER to further characterize monolayer tightness. Two tracer molecules are used: 70 kDa-Dextran-Tetramethyrhodamine (TMR) and 445 Da-Lucifer Yellow (LY) from Thermofisher. These tracers are dissolved in a transport buffer containing 0.1 g/l fetal bovine serum (FBS), 4.5 g/l glucose, and 10 mM HEPES in PBS. 100 μl of 10 mM TMR tracer dissolved in transport buffer is placed in the apical compartment of 3 replicate transwells of each cell type as well as a coated and uncoated control. The same is done in a separate well plate with 100 mM of LY in transport buffer. The basal compartments of all wells are filled with 600 μl of transport buffer. The plates are then incubated for a total of 1 hour while every 15 minutes, 100 μl are collected and replaced from the basal compartments of all wells. Tracer concentrations in the collected samples are measured using a CLARIOstar plus fluorescence microplate reader.

In an embodiment, permeability coefficients across the membranes with or without cells are calculated using the following equation EQ1:

$${P}_{s} = \frac{{C}_{b} {V}_{b}}{t A {C}_{a}} \qquad ( EQ1 )$$

C_{b} is the diffused tracer concentration in the basal compartment, V_{b} is the volume of the basal compartment, t is the time duration, A is membrane surface area between the compartments, and Cₐ is the loaded tracer concentration in the apical compartment.

Device Culture and BAFUS Treatment: In an embodiment, devices are syringe loaded with 10% collagen and incubated for at least 3 hours at 37° C. before culture is initiated. The collagen is then rinsed with PBS using a manual syringe. A syringe is then filled with a Caco-2 cell solution with a concentration of 1.5 million cells/ml. This allows for the 100 μl device channel volume to be filled with 150 k cells. The top channel of the devices is carefully filled with the cells while taking care not to introduce bubbles. Complete Caco-2 cell culture medium is loaded into the bottom channel while again trying to avoid bubbles. The devices are then placed in a Petri dish along with ~5 ml of PBS to maintain localized humidity and prevent excess evaporation from the devices. The devices are then cultured in static condition (i.e. without perfusion) for 24 hours.

In an embodiment, after 24 hours culture, permeabilities of 70K Dextran-TMR and LY are measured for each device. For a single permeability measurement, concentrated tracer solution (10 μM TMR or 100 μM LY) is loaded into the top channel of the devices and transport buffer is loaded into the bottom channels. The devices are then allowed to incubate at 37° C. for 15 minutes. The bottom channel 100 μl volumes are then collected and their concentrations are measured using the same methods used in previous transwell experiments. Permeability is then calculated using equation EQ1.

In an embodiment, after the initial permeability measure, the device's top channel is filled with 100×DBPC activated nanobubble solution and the bottom channel is filled with transport buffer. The device is then placed in the ultrasound tank apparatus and nanobubble solution is provided to the top channel at a constant flow of 15 ml/hr while BAFUS treatment is administered for a total of 2 minutes for each device. The subharmonic signal detected by the receiving transducer is monitored for the duration of the BAFUS treatment. Additional permeability measurements are made immediately after BAFUS treatment as well as after 24 hour cell culture at 37° C. inside a CO2 incubator to allow for recovery from the BAFUS treatment.

Apparatus Design: In an embodiment, an objective of the BAFUS BBB opening apparatus is to make it US-transparent with minimal energy scattering, reflection, and absorption so that accurate US power can be delivered to cell barriers. This could be achieved with an US window composed of thin membranes. FIG. 8A shows a schematic of an exemplary device design. In an embodiment, the device is composed of four PDMS frames: a top window frame (with holes for US to pass through the center and for inlets and outlets to the channels of the device), a bottom window frame (with a rectangular opening to allow for US to pass at any angle), a top channel frame (with a diamond opening that serves as the top channel of the device and two holes that serve as the inlet and outlet for the bottom channel), and finally a bottom channel frame (with just a diamond shaped opening that forms the bottom channel). The two channel frames are separated by a 12 μm porous polyester polytetrafluoroethylene (PETE) membrane where there is a 6 mm diameter overlapping region between the two channels. To contain the channels, each of the channel frames are sealed off by respective top and bottom 20 μm thick polydimethylsiloxane (PDMS) membranes.

In an embodiment, once the four frames and three thin membranes are assembled in the order shown in FIG. 8A, the US-transparent organ-on-chip device is interfaced with an epoxy barb to needle adaptor that us printed using a stereolithography (SLA) 3D printer (part 3 of FIG. 8B). The blunt ended needles are inserted into the inlets and outlets of the bottom and top channels; the barbs are used to quickly connect and disconnect the device from tubing when needed. The adaptor also has a large cross shaped opening to allow for US to pass freely at almost any angle to the device. The PDMS device with the adaptor is then placed in a custom milled clamp (1 and 2 of FIG. 8B) and secured by two screws to make a good liquid seal between the adaptor and the PDMS device. The clamp is designed with openings for the barbs to allow for enough room to connect inlet and outlet tubing. The top and bottom parts of the clamp are also designed to have large openings for FUS beam clearance. An image of the completely assembled US-transparent chip including the PDMS device, needle to barb adaptor, and the clamp is shown in FIG. 8C.

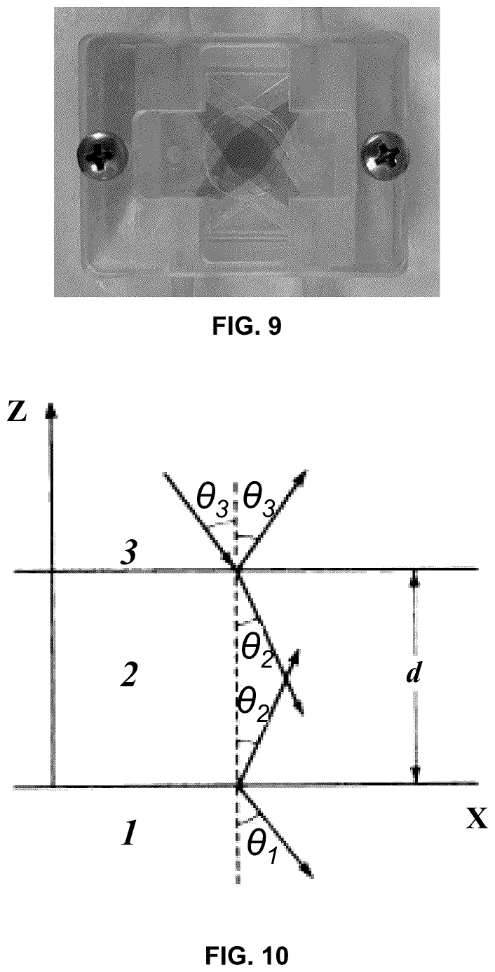

In an embodiment, after the devices are successfully fabricated, they are quality controlled by flowing DI water through them at a flow rate of 1.5 ml/s over 48 hours to ensure no leaks occur. With current molding supplies, 9 devices are fabricated at a time and consistently about 7/9 devices pass the quality control test. FIG. 9 shows an example of a leak free device that passed the test filled with colored dye. It clearly demonstrates the boundaries of the channels and the overlapping region in the middle of the device.

Ultrasound Transparency Calculation and Characterization: FIG. 10 shows a schematic of US transmission through a thin membrane in an embodiment, where d is the membranes thickness. In an embodiment, a theory of US passing through a thin membrane with similar materials on both sides of the membrane (same as in our chip situation) is used to assess the effects of the chip membrane on US transmission (20). The reflection coefficient of the membrane V can be expressed as:

$$V = ( {Z}_{2}^{2} - {Z}_{1}^{2} ) / ( {Z}_{1}^{2} + {Z}_{2}^{2} + 2 i {Z}_{1} {Z}_{2} cot {k}_{2 z} d ) \qquad ( EQ2 )$$

where the wave vector

$$k = \frac{ω}{c} = \frac{2 π * f}{c} ,$$

kₓ=k sin θ, k_{y}=0, k_{z}=k cos θ, the impedance

$${Z}_{i} = \frac{{ρ}_{i} {c}_{i}}{cos {θ}_{i}} ,$$

i=1,2,3 (Z₃=Z₁ for our situation), f and cᵢ are frequency and speeds of sound in the media, and θᵢ are the incident angles.

According to equation EQ2, V is a complex number with both a real and an imaginary component (Eqs. EQ3-EQ4). |V|² is considered for the magnitude of reflection (Eq. EQ5).

$${V}_{R e a l} = \frac{{Z}_{2}^{4} - {Z}_{1}^{4}}{( {Z}_{2}^{2} + {Z}_{1}^{2} ) + 4 {Z}_{1}^{2} {Z}_{2}^{2} {cot}^{2} {k}_{2 z} d} \qquad ( EQ3 )$$

$${V}_{Img} = \frac{2 i cot ( k d ) ( {Z}_{1}^{3} {Z}_{2} - {Z}_{2}^{3} {Z}_{1} )}{( {Z}_{2}^{2} + {Z}_{1}^{2} ) + 4 {Z}_{1}^{2} {Z}_{2}^{2} {cot}^{2} {k}_{2 z} d} \qquad ( EQ4 )$$

$${\vert V \vert}^{2} = {V}_{R e a l}^{2} + {V}_{Img}^{2} \qquad ( EQ5 )$$

The values of parameters used to calculate |V|² are provided in Tables 1 and 2 respectively. The actual angle of incidence for the ultrasound beam during BAFUS was 31° based on the orientation of the device to the transducer. Two other angles, 0° and 60°, were also calculated.

TABLE 1
List of known constants converted to standard units

| Constant | Value | Unit | SI Conversion | Converted Unit |
|---|---|---|---|---|
| f | 1 | MHz | 1000000 | Hz |
| c_{PDMS} | 1119 | m/s | 1119 | m/s |
| c_{PETE} | 1250 | m/s | 1250 | m/s |
| c_{H2O} | 1490 | m/s | 1490 | m/s |
| ρ_{PDMS} | 969 | kg/m³ | 969 | kg/m³ |
| ρ_{PETE} | 1.38 | g/cm³ | 1380 | kg/m³ |
| ρ_{H2O} | 1.053 | g/cm³ | 1053 | kg/m³ |
| PSMS membrane | 20 | μm | 0.00002 | m |
| thickness (d) | | | | |
| PETE membrane | 11 | μm | 0.000011 | m |
| thickness (d) | | | | |
| θ₃ (actual) | 31 | degrees | 0.541 | rad |
| θ₃ (test 1) | 60 | degrees | 1.047 | rad |
| θ₃ (test 2) | 0 | degrees | 0 | rad |

TABLE 2
List of calculated constants, equations, and values for three scenarios

| Calculated Constant and | Actual | Test 1 | Test 2 |
|---|---|---|---|
| | (theta3 = 31) | (theta3 = 60) | (theta3 = 0) |
| ${k}_{H 2 O} = \frac{2 π f}{{c}_{H 2 O}}$ | 4217 s²/m | 4217 s²/m | 4217 s²/m |
| ${Z}_{1} = \frac{{ρ}_{H 2 O} {c}_{H 2 O}}{cos ( {θ}_{3} )}$ | 1830413 Rayl | 3137940 Rayl | 1568970 Rayl |
| ${k}_{PDMS} = \frac{2 π f}{{c}_{PDMS}}$ | 5615 s²/m | 5615 s²/m | 5615 s²/m |
| ${θ}_{2 PDMS} = arcsin ( \frac{{k}_{H 2 O}}{{k}_{PDMS}} ) sin ( {θ}_{3} )$ | 0.3972 rad | 0.7081 rad | 0 rad |
| K_{2zPDMS} = kcos (θ_{2PDMS}) | 5178 s²/m | 4265 s²/m | 5615 s²/m |
| ${Z}_{2 PDMS} = {ρ}_{PDMS} * \frac{{c}_{PDMS}}{cos ( {θ}_{2 PDMS} )}$ | 1175832 Rayl | 1427477 Rayl | 1084311 Rayl |
| K_{PETE} | 5027 s²/m | 5027 s²/m | 5027 s²/m |
| θ_{2PETE} | 0.4468 rad | 0.8133 rad | 0 rad |
| K_{2ZPETE} | 4533 s²/m | 3454 s²/m | 5027 s²/m |
| Z_{2PETE} | 2012443 Rayl | 3450000 Rayl | 1725000 Rayl |

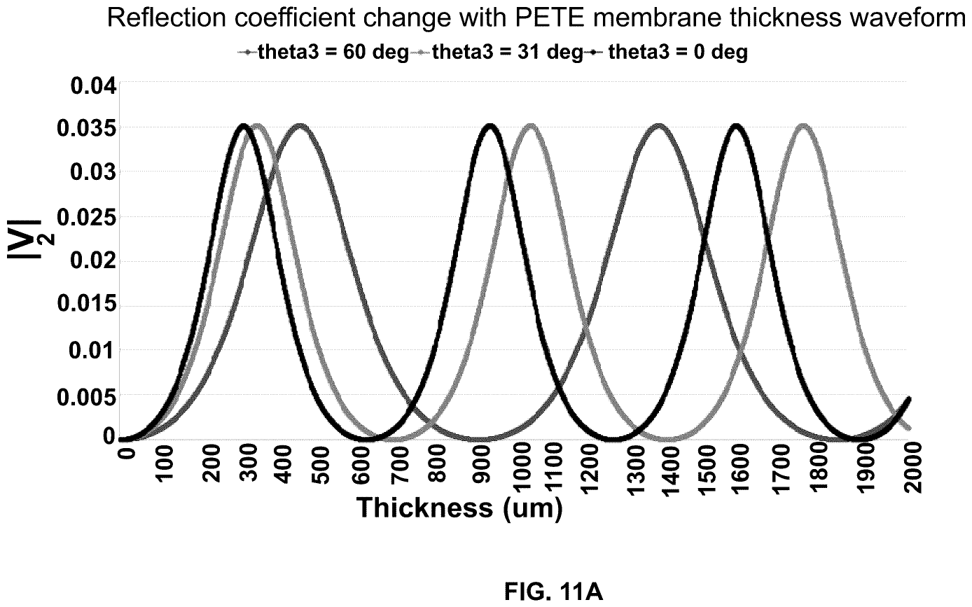

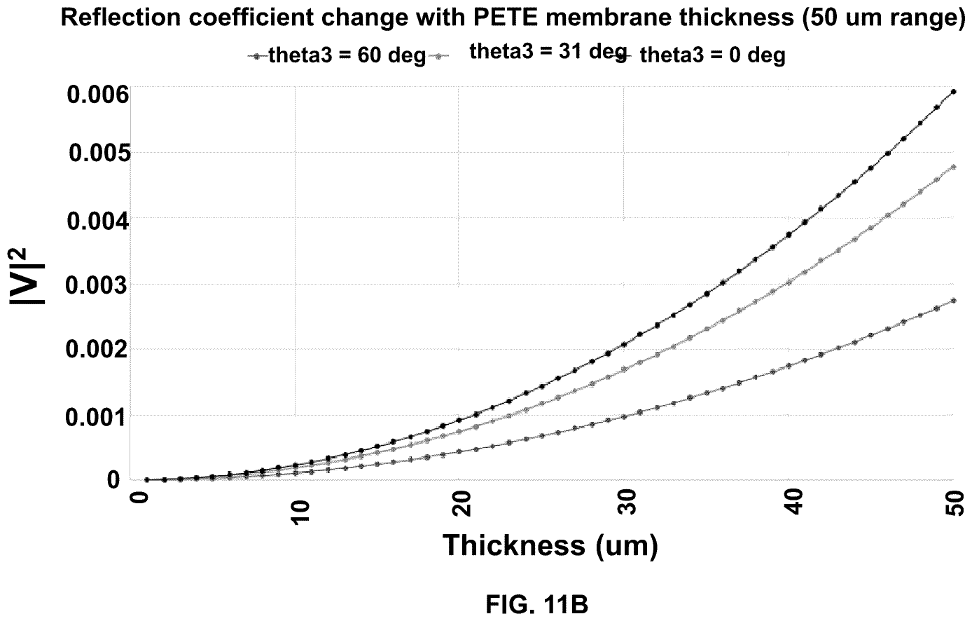

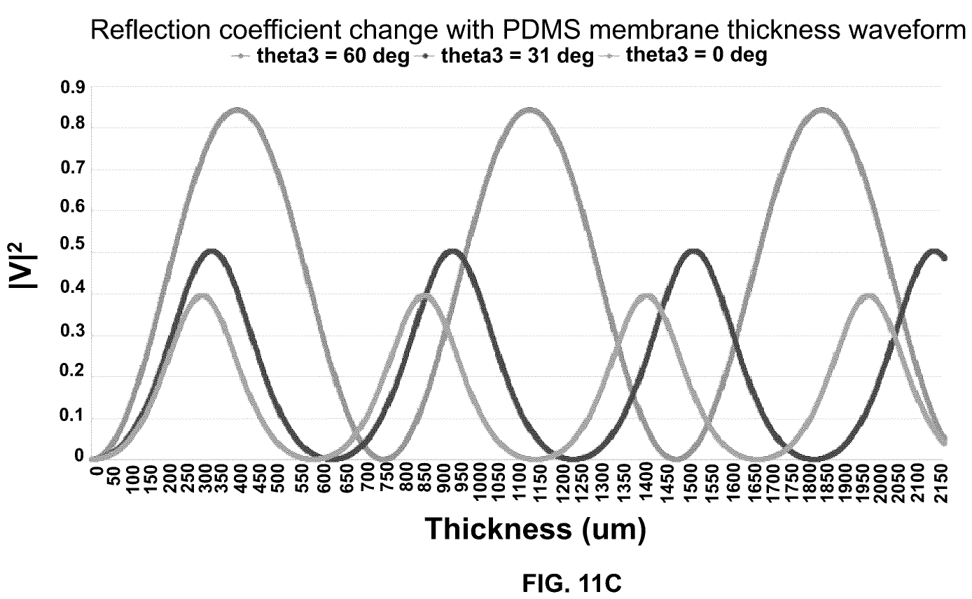

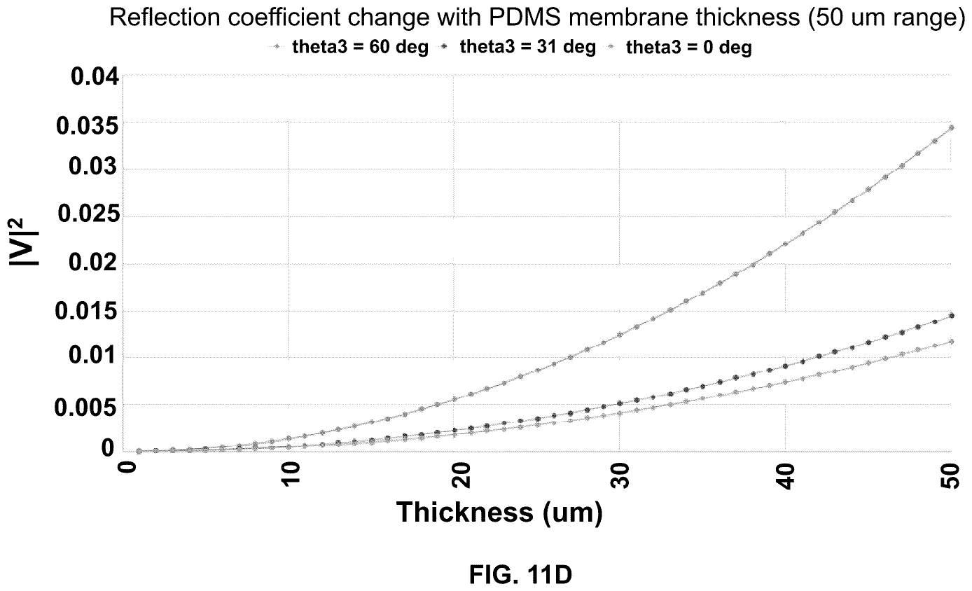

FIGS. 11A-11B show how US reflection changes with the membrane thickness for PETE and PDMS respectively. FIGS. 11C-11D are “Zoom-in” views of FIGS. 11A-11B for thin membrane thickness. They show that US reflections are extremely small (<0.05%) for both membranes at thin thicknesses (~10-20 μm). This is consistent with the theory that when

$$d \to 0 , V \to \frac{{Z}_{1} - {Z}_{3}}{{Z}_{1} + {Z}_{3}} = 0 ( 20 ) .$$

Furthermore, normal incident angle shows the highest reflection and reflection decreases with increasing incident angle. The reflection also changes periodically with a period of thickness ~500-900 μm. This can be explained by the “half-wave layer” effect, where the membrane would not have any effect on the incident wave (20). If absorption of the membrane is neglected, the transmission of US through the thin membrane can be expected to be:

$$T = 1 - {\vert V \vert}^{2} \to 1 \qquad ( EQ6 )$$

In an embodiment, the high transmission of a thin membrane to a FUS beam is experimentally validated using the setup shown in FIG. 6A-6C. A needle hydrophone is used to measure the acoustic peak pressures of a FUS beam at its focal point with (Pₚₑₐₖ′) and without (Pₚₑₐₖ) the chip's US-transparent window placed between the transducer and the hydrophone. The intensity of the sound I can be expressed by the sound pressure as:

$$I = \frac{{P}_{p e a k}^{2}}{2 ρ c} \qquad ( EQ 7 )$$

And the transmission of FUS beam through the device window can be expressed as:

$$T = \frac{{I}^{l}}{I} = {( \frac{{P}_{peak}^{′}}{{P}_{p e a k}} )}^{2} \qquad ( EQ 8 )$$

Tables 3-4 show the experiment results of Pₚₑₐₖ and Pₚₑₐₖ′ for both 0° and 31° incident angles. 6 devices were tested for each case. All device windows showed 100% transmission except two devices (92%) with 31° incident angle. This demonstrated the transparency of the device windows to FUS. The two 92% T could be due to misalignment of the FUS beam to the US-transparent window.

TABLE 3
Assessing FUS transmission through the device at 0° incident angle

| FUS Beam 0° to | | | |
|---|---|---|---|
| Device (#) | Pₚₑₐₖ (MPa) | Pₚₑₐₖ′ (MPa) | % Transmitted |
| 1 | 0.681 | 0.681 | 100 |
| 2 | 0.681 | 0.681 | 100 |
| 3 | 0.681 | 0.681 | 100 |
| 4 | 0.681 | 0.681 | 100 |
| 5 | 0.681 | 0.681 | 100 |
| 6 | 0.681 | 0.681 | 100 |

TABLE 4
Assessing FUS transmission through the device at a 31° incident angle

| FUS Beam 31° to | | | |
|---|---|---|---|
| Device (#) | Pₚₑₐₖ (MPa) | Pₚₑₐₖ′ (MPa) | % Transmitted |
| 1 | 0.377 | 0.362 | 92 |
| 2 | 0.377 | 0.362 | 92 |
| 3 | 0.362 | 0.362 | 100 |
| 4 | 0.362 | 0.362 | 100 |
| 5 | 0.362 | 0.362 | 100 |
| 6 | 0.348 | 0.348 | 100 |

### Finite Element Modeling of Organ-on-Chip Fluid Dynamics and Diffusion

Finite element modeling can help us better understand the fluid dynamics as well as the diffusion properties of different drugs in the Organ-On-Chip device. Knowing the fluid velocities and pressures at different locations in the channels can help device optimization to create the best possible environment to culture cells. Moreover, modeling diffusion of drugs across the membrane in the device could help our understanding on drugs traveling across the cellular barrier.

Laminar Flow Model: A first model, laminar flow, is based on the Navier Stokes equation, which can describe any type of flow be it laminar or turbulent given the application of the right terms. In an embodiment, the flow in the device is laminar because of the low Reynold number involved. The laminar flow resolved form of the Navier Stokes equation is provided below.

$${\underbrace{p ( \frac{\partial u}{\partial t} + u \cdot ∇ u )}}_{1} = {\underbrace{- ∇ p}}_{2} + {\underbrace{∇ \cdot ( μ ( ∇ u + {( ∇ u )}^{T} ) - \frac{2}{3} μ ( ∇ \cdot u ) I )}}_{3} + {\underbrace{F}}_{4} \qquad ( EQ 9 )$$

The equation can be described in four main terms as marked: (1) inertial forces, (2) pressure forces, (3) viscous forces, and (4) external forces applied to the fluid. Regarding the variables and constants, u is the fluid velocity, p is the fluid pressure, ρ is the fluid density, and μ is the dynamic viscosity of the fluid. It is also important to note that this model obeys the continuity principle as momentum is conserved and does not accumulate with time such that:

$$\frac{\partial ρ}{\partial t} + ∇ \cdot ( ρ u ) = 0 \qquad ( EQ 10 )$$

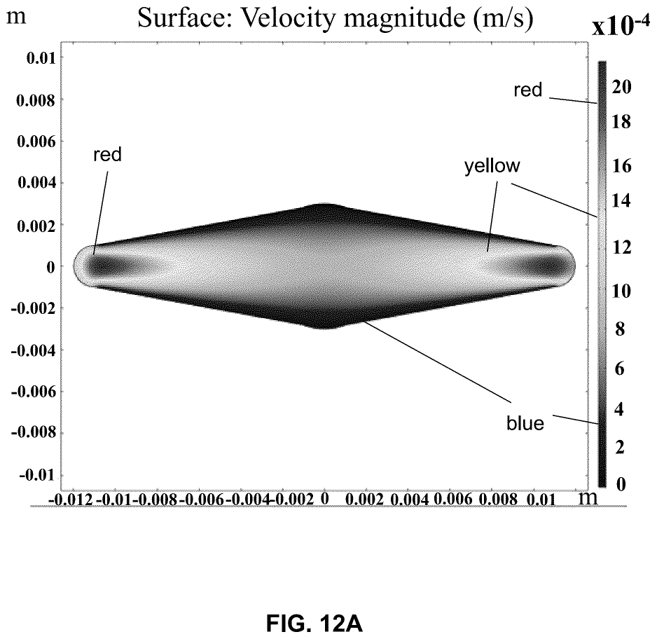

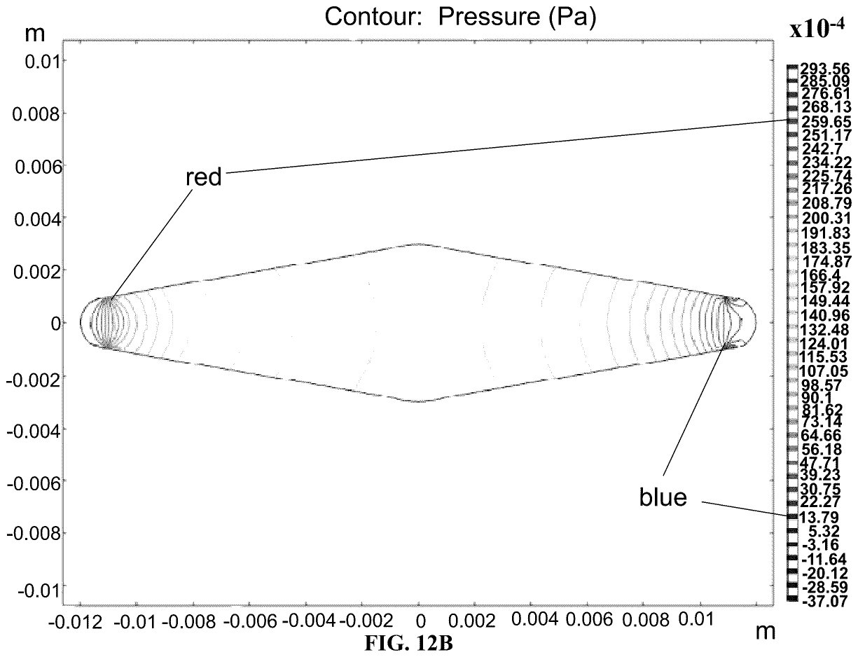

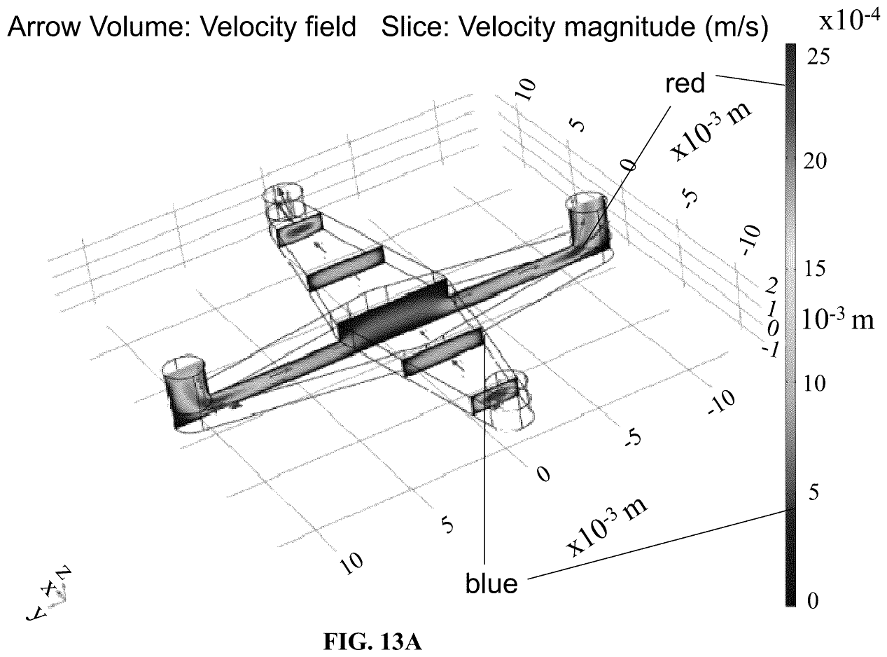

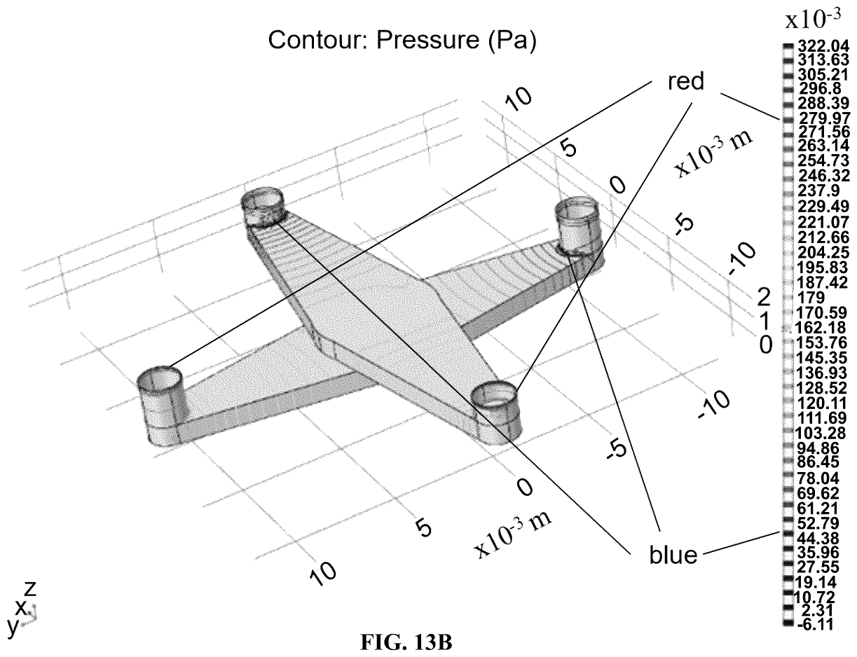

This model was used in two solutions, a 2D analysis of the y-plane of a single channel, and a 3D model of the entire device. An initial flow velocity set at the inlet boundary conditions was 1 mm/s. The solutions, velocity, and pressure profiles in 2D and 3D, are provided in FIGS. 12A-12B and FIGS. 13A-13B respectively.

The small flow velocities in the center window area seen in FIGS. 12A-12B and 13A-13B suggest that physiological shear in the BBB would not be achieved with the current chip geometries and the media circulation would simply be a matter of media replenishment. In fact, it is estimated that even without perfusion, the media in both channels in the device provide sufficient nutrients for a monolayer cell culture in the device for 24-48 hours, considering the fact that the medium thickness of 2 mm for the device is comparable to a standard culture flask medium thickness of ~2 mm. Therefore, for simplicity, static cell culture is used in this example for cultures shorter than 24-48 hours.

Transport of Diluted Species Model: A second model solves for the concentration profile in the device as fluorescent tracers pass through the porous membrane, in an embodiment. The device geometry was just like the one shown in FIGS. 7A-7C, with an added component, i.e. a thin diffusion barrier between the top and bottom channels. There are two crucial parameters that characterize this thin diffusion barrier in COMSOL MULTIPHYSICS® software: the membrane thickness dₛ and the effective diffusion coefficient across the membrane Dₑᵢ that can be calculated by the following equation taken from a study on filter membrane characterization (17).

$${D}_{ei} = \frac{ε {D}_{i}}{τ} {K}_{ri} {K}_{r} = {[ 1 - \frac{{d}_{m}}{{d}_{p}} ]}^{4} , \frac{{d}_{m}}{{d}_{p}} \leq 1 \qquad ( EQ 11 )$$

where ε is the membrane porosity, Dᵢ is the diffusivity of the tracer, τ is the tortuosity, Kᵣ is the restrictive factor, dₘ is the size of the molecule, dₚ is the size of the membrane pore (18). τ has been approximated as 1 for track etched membrane. ε was taken from the SterilTech membrane manufacturer's site to be 0.3%. The Dᵢ and dₘ for 70 kDa-dextran, a common fluorescent tracer molecule used to mimic certain chemotherapeutic drugs in permeability studies, were estimated to be 13.9*10⁻⁸ cm²/s and 3 nm, which leads to a Dₑᵢ value of 4.046*10⁻¹⁰ cm²/s.

In an embodiment, diffusion is similar both across the membrane pore and in the device channels. The equation describing the transport of diluted species model is provided:

$$∇ \cdot - D ∇ c + u \cdot ∇ c = R , R = 0 \qquad ( EQ12 )$$

In this case, the diffusion coefficient D is of dextran in water or cell media. Fluid velocity u is a gradient based on initial flow rates and position. Finally, the reaction term R is zero in this model because it is steady state and there is no generation or degradation of dextran.

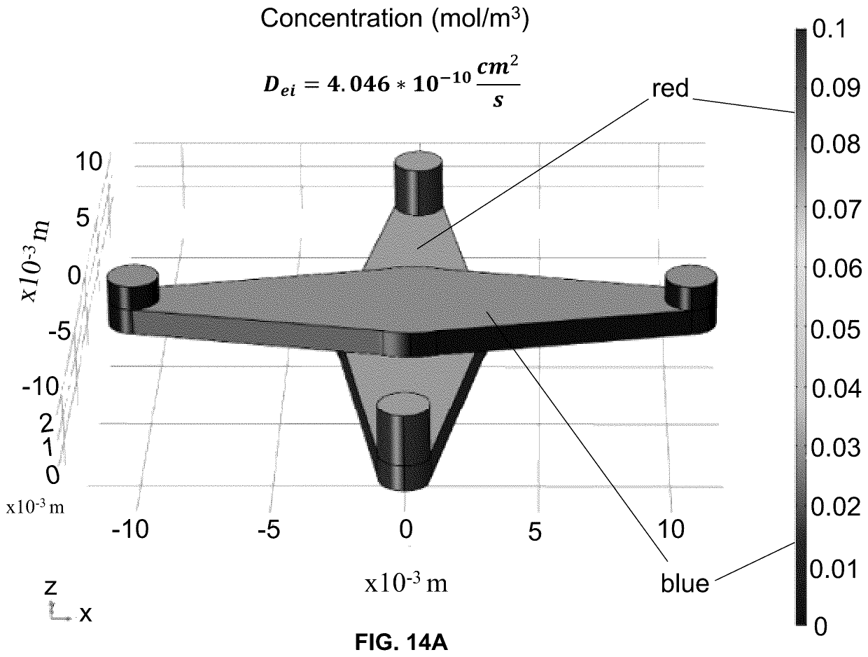

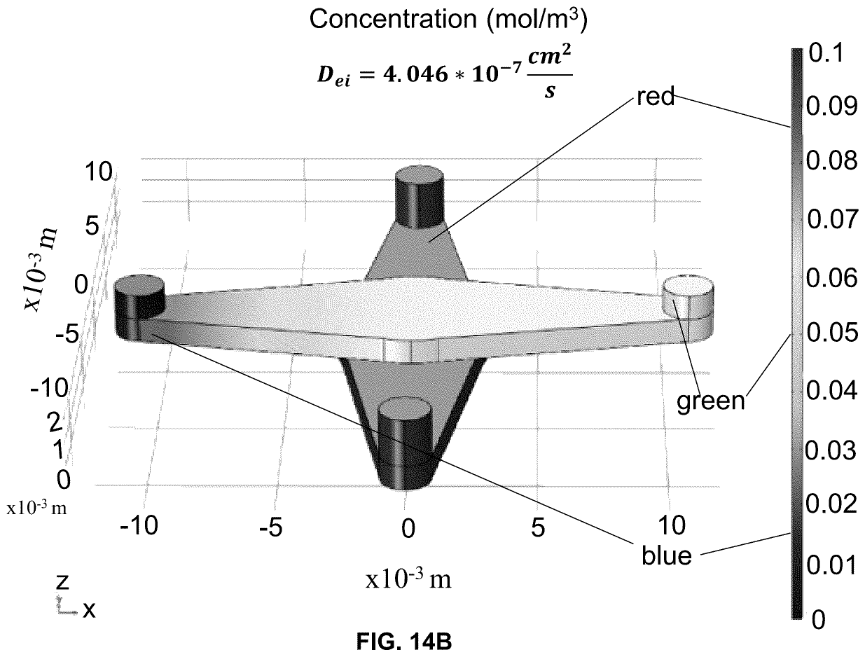

In an embodiment, multiple 3D solutions to this model are generated in COMSOL MULTIPHYSICS® for different membrane diffusion constants in attempt to model different states of the organ-on-chip device. The bottom channel is defined with a known concentration of 100 μM and the top channel has an inlet with an initial 1 mm/s flow velocity and an opposing outlet. The calculated diffusivity of the membrane is a good model of a device with no cultured cells at all where convection could occur through the membrane's pores. FIG. 14A shows the results using parameters for 70 kDa Dextran estimated above. Minimal tracer is observed across the membrane. However, when Dₑᵢ is increased 1000× (such as LY across a 10% porosity membrane), clear tracer diffusion across the membrane and convection in the top channel are observed, which correctly reflects the physics of the tracer in the device.

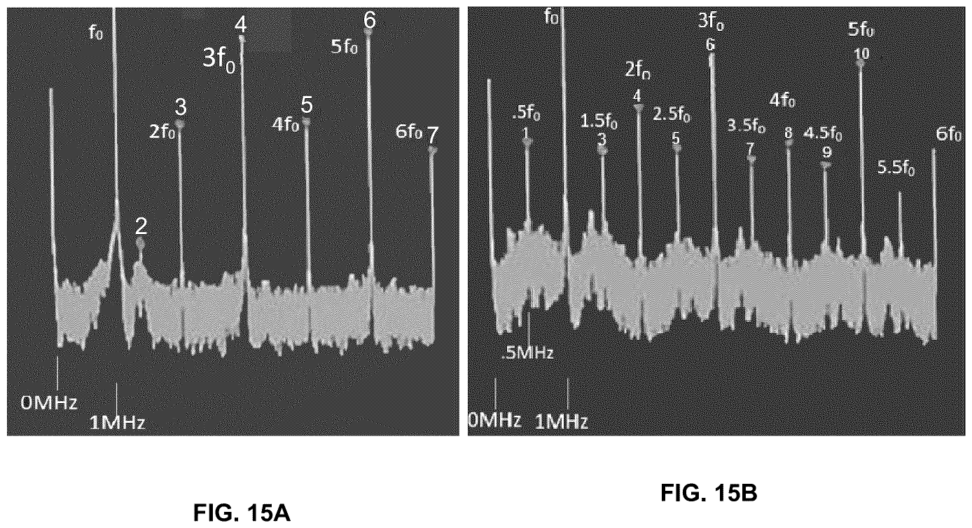

Nanobubble Characterization: In an embodiment, nanobubbles are fabricated as described earlier. To demonstrate the existence of nanobubbles, diluted bubble solution in PBS is loaded into the organ-on-chip device and the BAFUS setup described in FIG. 2 and above is used to excite the bubbles. The acoustic signal of bubble cavitation is monitored using the receiving US transducer, and FFT of the signal is displayed by a digital storage oscilloscope. FIGS. 15A-15B show the FFT images of acoustic monitoring of PBS and the DBPC 100× nanobubble solutions loaded into the device. It is clearly seen that PBS only produces fundamental and harmonic peaks (1, 2, 3 . . . . MHz) for the 1 MHz FUS excitation pulse; on the other hand, sub- and super-harmonic peaks (0.5, 1.5, 2.5 . . . . MHz) are observed for the nanobubble solution. Sub- and super-harmonic peaks are characteristic nonlinear signals from bubble cavitation. The observation of these peaks clearly demonstrates the existence of the bubbles in the nanobubble solution.

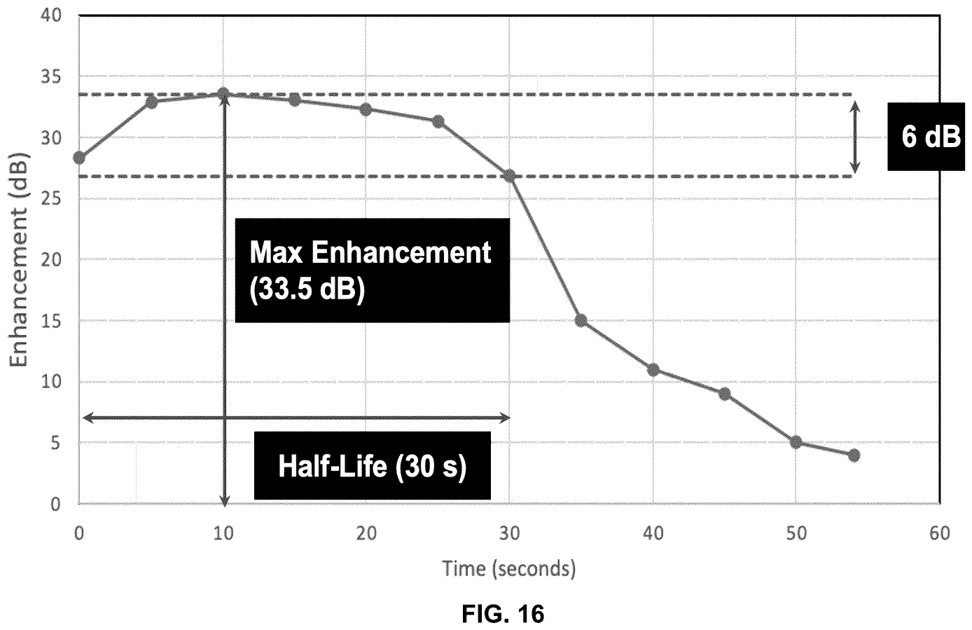

In an embodiment, next, the sub-harmonic peak (0.5 MHz) is used to characterize the cavitation signal and lifetime of the bubbles. A static condition is tested first, i.e. the nanobubble solution is kept in place after loading into the device and the FUS pulses are applied. FIG. 16 shows a typical plot of how the sub-harmonic peak height (labeled as signal Enhancement) changes over time for a single loading of a 100×DBPC solution. The decrease of the signal back to the baseline indicates the consumption of the bubbles. The half-life of the bubble solution is defined by a 6 dB decrease from the maximum signal enhancement.

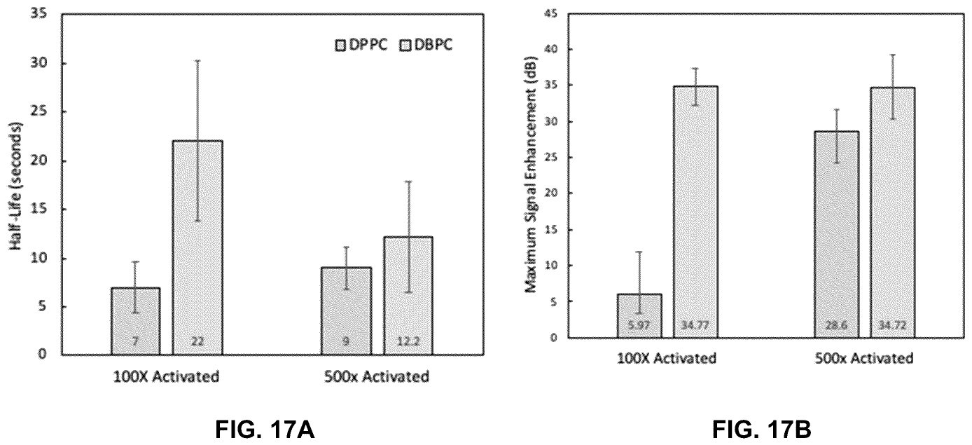

In an embodiment, using this methodology, nanobubble solutions of different recipes and dilution factors are characterized according to their maximum signal enhancements and half-lives. FIGS. 17A-17B show that the DBPC recipe has a stronger cavitation signal and longer half-lives than the DPPC recipe. The 100× and 500× diluted DBPC solutions show similar maximum sub-harmonic peak heights, but the 100× diluted solution has longer half-life than the 500× diluted solution. Based on these observations, 100× DBPC nanobubble solution is used in the following BAFUS barrier disruption experiments.

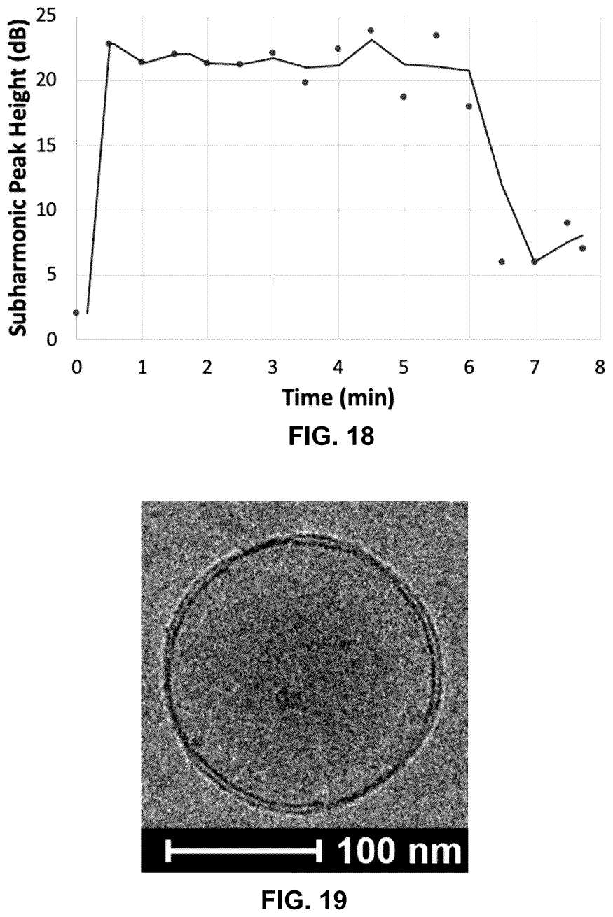

In an embodiment, further characterizations are also performed for the 100× DBPC nanobubbles under a constant flow (15 ml/hr) condition. FIG. 18 shows that the maximum sub-harmonic peak can be maintained for more than 5 min, which offers plenty of time for BAFUS therapy to be administered.

In an embodiment, finally, as shown in FIG. 19, Cryo-TEM images are also obtained for the bubbles created using the 100×DBPC recipe. Bubbles similar to those reported in literature, i.e. ~200 nm in diameter with lipid bilayer shells and a darkened core, are observed (25).

Transwell Culture Optimization: Transwells are quick and easy models that resemble culture conditions in the device. In transwells, cells grow on PETE membranes with the same 0.4 μm pore size as the membrane in the Organ-on-Chip device. In an embodiment, before optimizing cell culture in the device, transwell culture is performed to optimize cellular barriers using permeability and TEER measurements.

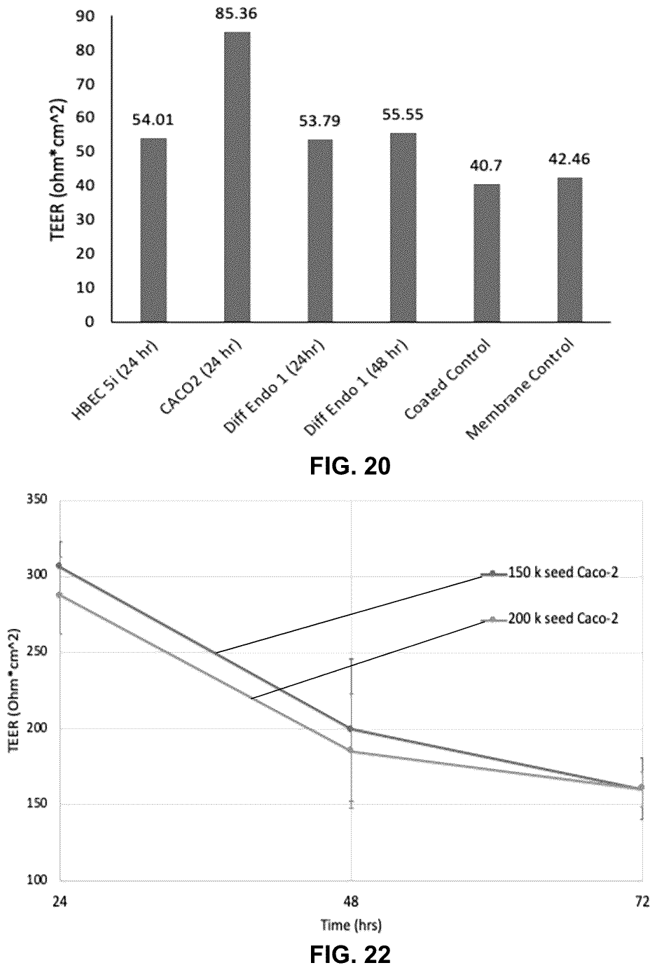

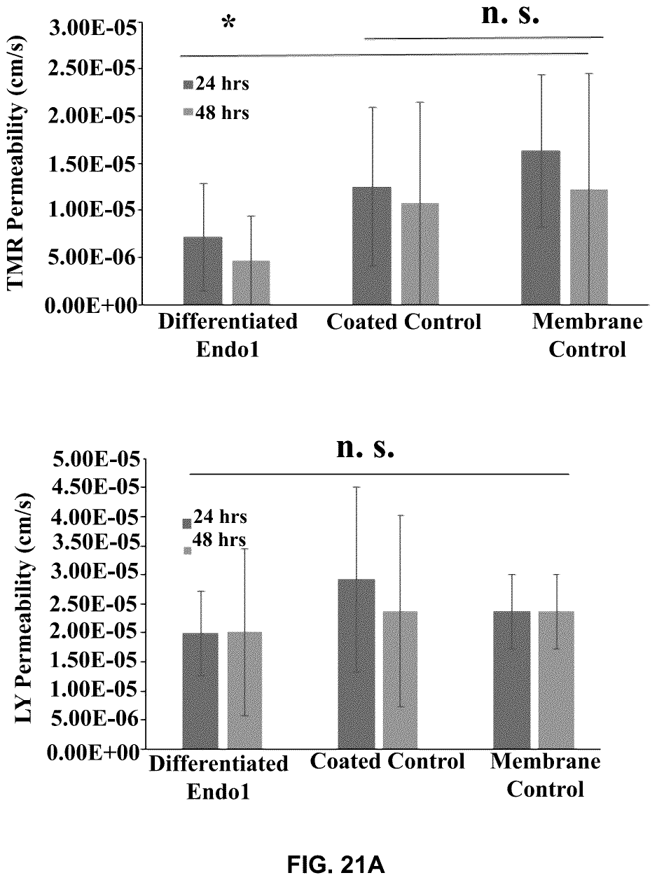

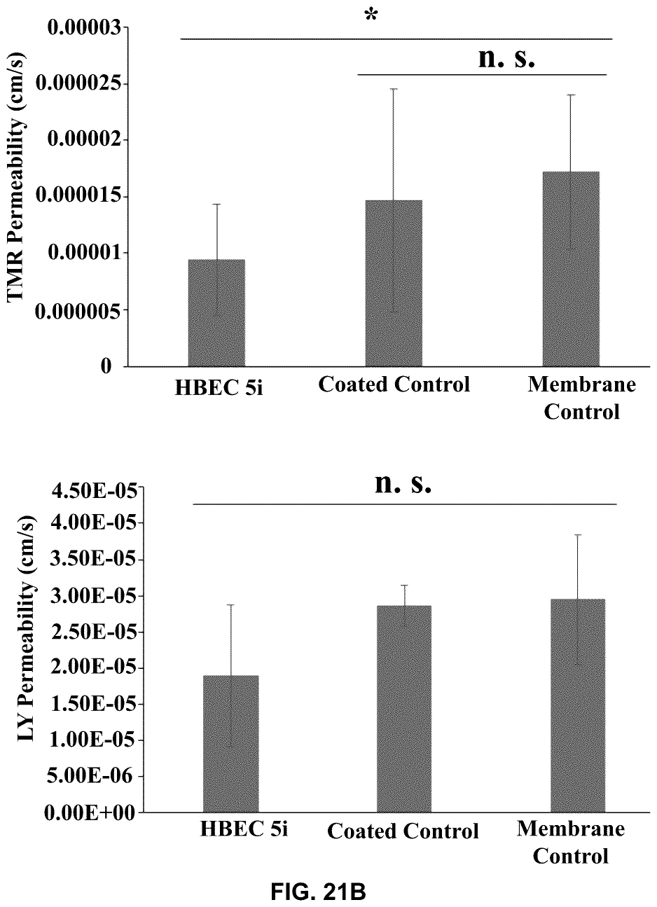

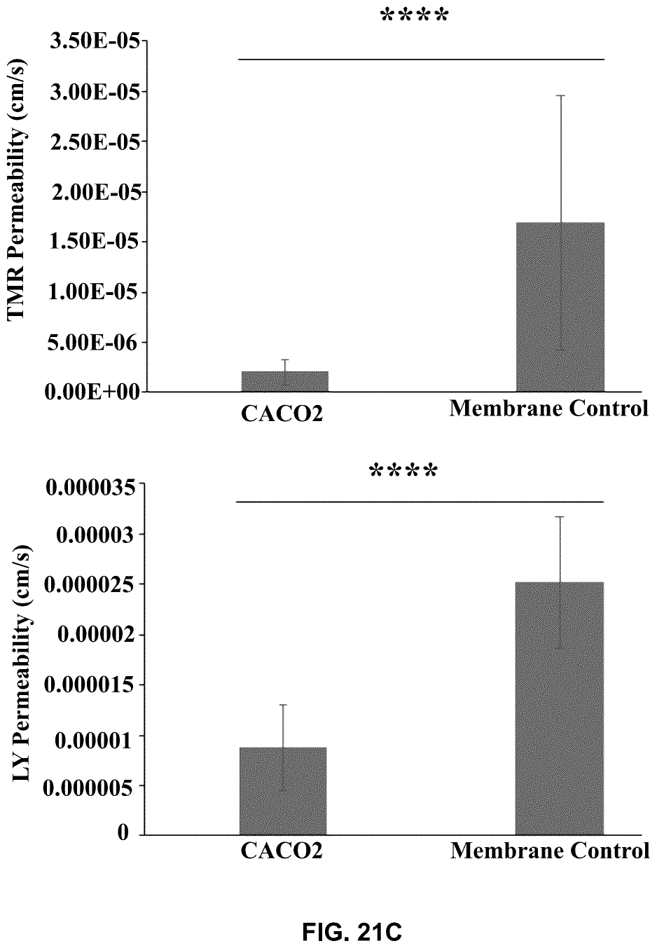

Cell Type Selection from a Panel of Cells: In an embodiment, three cell types (Caco-2, Endo-1, and HBEC-5i) are tested for their ability to form tight cellular barriers. FIG. 20 shows TEER measurements of preliminary barrier formation, with Caco-2 cells achieving the highest TEER in just 24 hours. During this experiment the Caco-2 cells and Endo-1 cells begin detaching after the 24-hour measurements are made, specifically after changing media. This is attributed to the fact that no collagen coating is applied to the Caco-2 culture surfaces and that the gelatin used to coat the remaining wells is faulty. Collagen is used in later Caco-2 culture optimization experiments. FIGS. 21A-21C further confirm that Caco-2 cells achieve permeabilities that re significantly lower than other barriers (HBEC-5i and Endo-1 cells) for LY and TMR tracers. These results lead us to choose Caco-2 cells for BAFUS barrier disruption studies.

Optimization of Caco-2 Culture: In an embodiment, the Caco-2 cell culture protocol is further optimized based on literature to obtain better barrier properties for BAFUS barrier disruption study. Literature has shown that high seeding density can lead to high TEER values (150-400 ohms*cm2) in just 24-72 hours (28, 29). The protocols mentioned in these studies also used a 10% collagen solution coating on the substrate. In an embodiment, these conditions are tested. FIG. 22 shows that indeed, TEER values of approximately 300 ohm*cm2 are achieved within 24 hours for seeding densities of 150 k and 200 k cells/insert. There is a general decrease in TEER over the three-day culture period for both seeding densities, indicating an optimal culture period of 24 hours for BAFUS treatment.

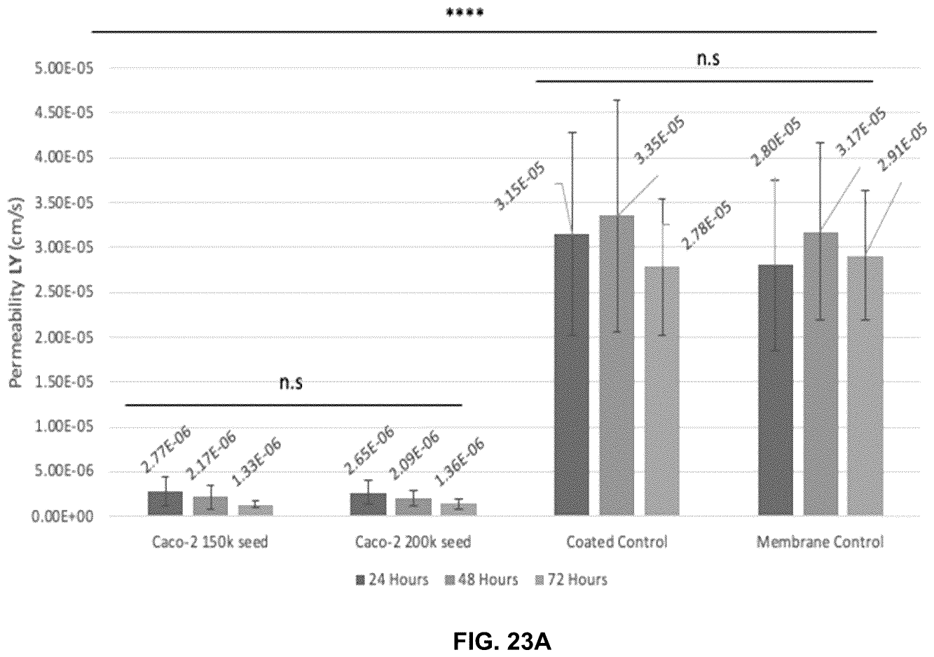

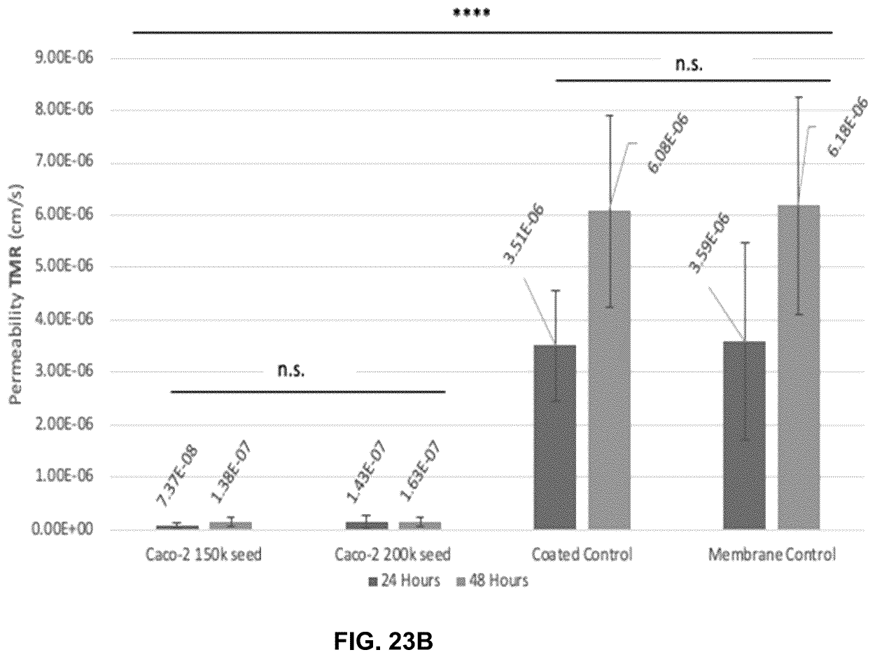

FIGS. 23A-23B show that in an embodiment Caco-2 barriers from both seeding densities significantly decrease the permeabilities to TMR and LY tracers at 24 and 48 hour time points, and no significant difference between the two seeding densities is observed. Based on these observations, the 150 k cell/insert is chosen for further studies as it requires less cells.

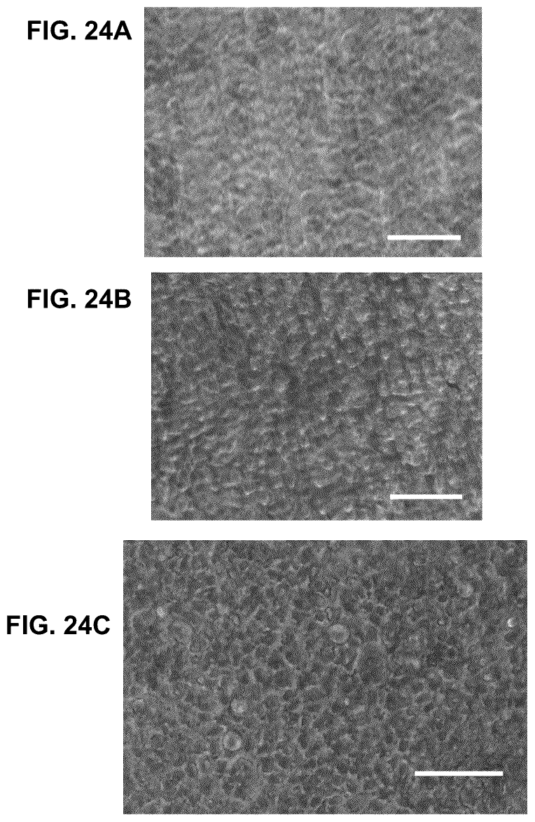

Caco-2 Barrier in US-transparent Chip and BAFUS Barrier Disruption: In an embodiment, after transwell barrier optimization, 150 k Caco-2 cells are loaded into the chip device to form the barrier. Cellular barrier is visible at 24 hours by phase contrast microscopy, as shown in FIG. 24A (baseline). Then 100×DBPC solution is flowed through the device and BAFUS treatment is applied using 1 MHz pulses (100 m Vrms, 10 k cycles/pulse and 1 Hz pulse frequency) for 2 mins. FIGS. 24B-24C show that the phase contrast microscopy images of the cellular barrier immediately and 24 hrs after BAFUS treatment, respectively. No significant damage of the barrier is visible.

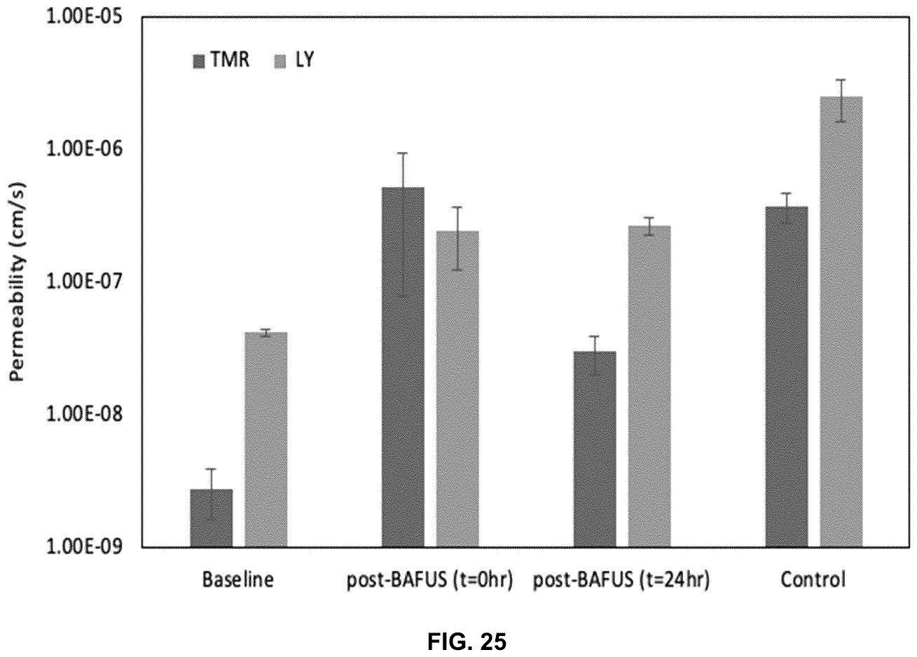

Even though similar phase contrast images were obtained, permeability of the barrier to molecular tracer could change dramatically after the BAFUS treatment. FIG. 25 shows that in an embodiment a 100-fold increase in permeability to the 70 k-Dextran-TMR tracer from baseline is observed immediately after the BAFUS treatment, demonstrating the disruption of the barrier property. This disruption is observed to be recovered by culturing the cellular barrier over time, as shown by the 10-fold decrease of the permeability after 24 h of barrier culture post BAFUS treatment.

In an embodiment, even though clear barrier disruption and recovery are observed for the 70 k-Dextran-TMR tracer, the difference between LY tracer permeability post BAFUS and post recovery culture is not so pronounced. This could be due to the much smaller molecular size of LY and the differentiate tightness of the cellular barrier for different sized molecular tracers. It could take a longer culture period to restore baseline barrier tightness and observe a more pronounced difference between LY post-BAFUS and post-recovery permeability measurements.

US-transparency is a feature of an embodiment of our Organ-on-Chip platform that enables quantitative linkage between BAFUS power by different parameters and the disruption of the cellular barrier. In an embodiment, US-transparent organ-on-chip devices based on thin membranes are designed and fabricated. In an embodiment, the US transparency of the thin membrane window of the device is theoretically calculated and verified by experiments. Nanobubbles are fabricated and showed bubble cavitation inside the device, as demonstrated by the sub- and super-harmonic peaks from acoustic monitoring. In an embodiment, FEM is used to understand the flow profile and molecular diffusion inside the device. In an embodiment, a fast Caco-2 barrier formation protocol is demonstrated based on literature, and barrier disruption by BAFUS and subsequent recovery are observed in Caco-2 seeded US-transparent chip device.

### References for Example 1

1. Wolff, A., Antfolk, M., Brodin, B., & Tenje, M. (2015). In Vitro Blood-Brain Barrier Models—An Overview of Established Models and New Microfluidic Approaches. Journal of Pharmaceutical Sciences, 104(9), 2727-2746. https://doi.org/10.1002/jps.24329
2. Pardridge, W. M. (2012). Drug transport across the blood-brain barrier. Journal of Cerebral Blood Flow & Metabolism, 32(11), 1959-1972. https://doi.org/10.1038/jcbfm.2012.126
3. Abrahao, A., Meng, Y., Llinas, M., Huang, Y., Hamani, C., Mainprize, T., Aubert, I., Heyn, C., Black, S. E., Hynynen, K., Lipsman, N., & Zinman, L. (2019). First-in-human trial of blood-brain barrier opening in amyotrophic lateral sclerosis using MR-guided focused ultrasound. Nature Communications, 10(1), 4373. https://doi.org/10.1038/s41467-019-12426-9
4. Izadifar, Z., Babyn, P., & Chapman, D. (2019). Ultrasound Cavitation/Microbubble Detection and Medical Applications. Journal of Medical and Biological Engineering, 39(3), 259-276. https://doi.org/10.1007/s40846-018-0391-0
5. Aryal, M., Arvanitis, C. D., Alexander, P. M., & McDannold, N. (2014). Ultrasound-mediated blood-brain barrier disruption for targeted drug delivery in the central nervous system. Advanced Drug Delivery Reviews, 0, 94-109. https://doi.org/10.1016/j.addr.2014.01.008
6. Norén, E., Mellander, M.-R., Almer, S., & Söderman, J. (2018). Genetic Variation and Gene Expression Levels of Tight Junction Genes Indicates Relationships Between PTEN as well as MAGI1 and Microscopic Colitis. Digestive Diseases and Sciences, 63(1), 105-112. https://doi.org/10.1007/s10620-017-4857-7
7. Yamaura, Y., Chapron, B. D., Wang, Z., Himmelfarb, J., & Thummel, K. E. (2016). Functional Comparison of Human Colonic Carcinoma Cell Lines and Primary Small Intestinal Epithelial Cells for Investigations of Intestinal Drug Permeability and First-Pass Metabolism. Drug Metabolism and Disposition, 44(3), 329-335. https://doi.org/10.1124/dmd.115.068429
8. Endo-Takahashi, Y., & Negishi, Y. (2020). Microbubbles and Nanobubbles with Ultrasound for Systemic Gene Delivery. Pharmaceutics, 12(10), 964. https://doi.org/10.3390/pharmaceutics12100964
9. Zhang, D. Y., Dmello, C., Chen, L., Arrieta, V. A., Gonzalez-Buendia, E., Kane, J. R., Magnusson, L. P., Baran, A., James, C. D., Horbinski, C., Carpentier, A., Desseaux, C., Canney, M., Muzzio, M., Stupp, R., & Sonabend, A. M. (2020). Ultrasound-mediated Delivery of Paclitaxel for Glioma: A Comparative Study of Distribution, Toxicity, and Efficacy of Albumin-bound Versus Cremophor Formulations. Clinical Cancer Research, 26(2), 477-486. https://doi.org/10.1158/1078-0432.CCR-19-2182
10. Greish, K. (2010). Enhanced Permeability and Retention (EPR) Effect for Anticancer Nanomedicine Drug Targeting. In S. R. Grobmyer & B. M. Moudgil (Eds.), Cancer Nanotechnology: Methods and Protocols (pp. 25-37). Humana Press. https://doi.org/10.1007/978-1-60761-609-2_3
11. Peyman, S. A., McLaughlan, J. R., Abou-Saleh, R. H., Marston, G., Johnson, B. R. G., Freear, S., Coletta, P. L., Markham, A. F., & Evans, S. D. (2016). On-chip preparation of nanoscale contrast agents towards high-resolution ultrasound imaging. Lab on a Chip, 16(4), 679-687. https://doi.org/10.1039/C5LC01394A
12. Ebina, K., Shi, K., Hirao, M., Hashimoto, J., Kawato, Y., Kaneshiro, S., Morimoto, T., Koizumi, K., & Yoshikawa, H. (2013). Oxygen and Air Nanobubble Water Solution Promote the Growth of Plants, Fishes, and Mice. PLOS ONE, 8(6), e65339. https://doi.org/10.1371/journal.pone.0065339
13. Brain Tumor—Statistics. (2012 Jun. 25). Cancer. Net. https://www.cancer.net/cancer-types/brain-tumor/statistics
14. American Cancer Society|Information and Resources about for Cancer: Breast, Colon, Lung, Prostate, Skin. (n.d.). Retrieved Jan. 28, 2022, from https://www.cancer.org/cancer/brain-spinal-cord-tumors-adults/detection-diagnosis-staging/survival-rates.html.
15. AZ 1512 HS. (n.d.). EPFL. Retrieved Feb. 17, 2022, from https://www.epfl.ch/research/facilities/cmi/process/photolithography/photoresist-selection/az-1512-hs/
16. Baëtens, T., & Arscott, S. (2019). Planarization and edge bead reduction of spin-coated polydimethylsiloxane. Journal of Micromechanics and Microengineering, 29(11), 115005. https://doi.org/10.1088/1361-6439/ab3b18
17. Kruczek, B. (2015). Diffusion Coefficient. Encyclopedia of Membranes, 1-4. https://doi.org/10.1007/978-3-642-40872-4_1993-1
18. Alkhudhiri, A., & Hilal, N. (2018). Membrane distillation—Principles, applications, configurations, design, and implementation. Emerging Technologies for Sustainable Desalination Handbook, 55-106. https://doi.org/10.1016/b978-0-12-815818-0.00003-5
19. DeStefano, J. G., Jamieson, J. J., Linville, R. M., & Searson, P. C. (2018). Benchmarking in vitro tissue-engineered blood-brain barrier models. Fluids and Barriers of the CNS, 15(1). https://doi.org/10.1186/s12987-018-0117-2.
20. Brekhovskikh, L. (2012). Waves in Layered Media. Elsevier.
21. Wang, H., Cao, W., Zhou, Q., & Shung, K. K. (2003). Characterization of ultra-thin quarter-wavelength matching layers of high frequency ultrasonic transducers. IEEE Symposium on Ultrasonics, 2003, 1, 1048-1051 Vol. 1. https://doi.org/10.1109/ULTSYM.2003.1293579
22. Lapin, N. A., Gill, K., Shah, B. R., & Chopra, R. (2020). Consistent opening of the blood brain barrier using focused ultrasound with constant intravenous infusion of microbubble agent. Scientific Reports, 10 (1), 16546. https://doi.org/10.1038/s41598-020-73312-9
23. Cheng, B., Bing, C., Xi, Y., Shah, B., Exner, A. A., & Chopra, R. (2019). Influence of Nanobubble Concentration on Blood-Brain Barrier Opening Using Focused Ultrasound Under Real-Time Acoustic Feedback Control. Ultrasound in Medicine & Biology, 45(8), 2174-2187. https://doi.org/10.1016/j.ultrasmedbio.2019.03.016
24. Thompson, R. F., Walker, M., Siebert, C. A., Muench, S. P., & Ranson, N. A. (2016). An introduction to sample preparation and imaging by cryo-electron microscopy for structural biology. Methods (San Diego, Calif.), 100, 3-15. https://doi.org/10.1016/j.ymeth.2016.02.017
25. de Leon, A., Perera, R., Hernandez, C., Cooley, M., Jung, O., Jeganathan, S., Abenojar, E., Fishbein, G., Sojahrood, A. J., Emerson, C. C., Stewart, P. L., Kolios, M. C., & Exner, A. A. (2019). Contrast enhanced ultrasound imaging by nature-inspired ultrastable echogenic nanobubbles. Nanoscale, 11(33), 15647-15658. https://doi.org/10.1039/C9NR04828F
26. Streeter, I., Harrison, P. W., Faulconbridge, A., The HipSci Consortium, Flicek, P., Parkinson, H., & Clarke, L. (2017). The human-induced pluripotent stem cell initiative—Data resources for cellular genetics. Nucleic Acids Research, 45(D1), D691-D697. https://doi.org/10.1093/nar/gkw928
27. Neal, E. H., Marinelli, N. A., Shi, Y., McClatchey, P. M., Balotin, K. M., Gullett, D. R., Hagerla, K. A., Bowman, A. B., Ess, K. C., Wikswo, J. P., & Lippmann, E. S. (2019). A Simplified, Fully Defined Differentiation Scheme for Producing Blood-Brain Barrier Endothelial Cells from Human iPSCs. Stem Cell Reports, 12(6), 1380-1388. https://doi.org/10.1016/j.stemcr.2019.05.008
28. Yamashita, S., Konishi, K., Yamazaki, Y., Taki, Y., Sakane, T., Sezaki, H., & Furuyama, Y. (2002). New and better protocols for a short-term Caco-2 cell culture system. Journal of Pharmaceutical Sciences, 91(3), 669-679. https://doi.org/10.1002/jps.10050
29. Lea, T. (2015). Caco-2 Cell Line. In K. Verhoeckx, P. Cotter, I. López-Expósito, C. Kleiveland, T. Lea, A. Mackie, T. Requena, D. Swiatecka, & H. Wichers (Eds.), The Impact of Food Bioactives on Health (pp. 103-111). Springer International Publishing. https://doi.org/10.1007/978-3-319-16104-4_10
30. Cucullo, L., Couraud, P.-O., Weksler, B., Romero, I.-A., Hossain, M., Rapp, E., & Janigro, D. (2008). Immortalized human brain endothelial cells and flow-based vascular modeling: A marriage of convenience for rational neurovascular studies. Journal of Cerebral Blood Flow and Metabolism: Official Journal of the International Society of Cerebral Blood Flow and Metabolism, 28(2), 312-328. https://doi.org/10.1038/sj.jcbfm.9600525
31. DeStefano, J. G., Xu, Z. S., Williams, A. J., Yimam, N., & Searson, P. C. (2017). Effect of shear stress on iPSC-derived human brain microvascular endothelial cells (dhBMECs). Fluids and Barriers of the CNS, 14(1), 20. https://doi.org/10.1186/s12987-017-0068-z
32. Helms, H. C., Abbott, N. J., Burek, M., Cecchelli, R., Couraud, P.-O., Deli, M. A., Förster, C., Galla, H. J., Romero, I. A., Shusta, E. V., Stebbins, M. J., Vandenhaute, E., Weksler, B., & Brodin, B. (2016). In vitro models of the blood-brain barrier: An overview of commonly used brain endothelial cell culture models and guidelines for their use. Journal of Cerebral Blood Flow and Metabolism: Official Journal of the International Society of Cerebral Blood Flow and Metabolism, 36(5), 862-890. https://doi.org/10.1177/0271678X16630991
33. Deosarkar S P, Prabhakarpandian B, Wang B, Sheffield J B, Krynska B, et al. (2015) A Novel Dynamic Neonatal Blood-Brain Barrier on a Chip. PLOS ONE 10(11): e0142725. https://doi.org/10.1371/journal.pone.0142725
34. Lelu, S., Afadzi, M., Berg, S., Aslund, A. K., Torp, S. H., Sattler, W., & de L Davies, C. (2017). Primary Porcine Brain Endothelial Cells as In Vitro Model to Study Effects of Ultrasound and Microbubbles on Blood-Brain Barrier Function. IEEE transactions on ultrasonics, ferroelectrics, and frequency control, 64(1), 281-290. https://doi.org/10.1109/TUFFC.2016.2597004
35. Blomley, M. J. K., Cooke, J. C., Unger, E. C., Monaghan, M. J., & Cosgrove, D. O. (2001). Microbubble contrast agents: A new era in ultrasound. BMJ, 322(7296), 1222-1225. https://doi.org/10.1136/bmj.322.7296.1222
36. Miller M W. Gene transfection and drug delivery. Ultrasound Med Biol. 2000; 26 Suppl 1: S59-S62. doi: 10.1016/S0301-5629(00)00166-6.
37. Tsutsui, J. M., Xie, F., & Porter, R. T. (2004). The use of microbubbles to target drug delivery. Cardiovascular Ultrasound, 2, 23. https://doi.org/10.1186/1476-7120-2-23
38. Konofagou, E. E., Tung, Y.-S., Choi, J., Deffieux, T., Baseri, B., & Vlachos, F. (2012). Ultrasound-Induced Blood-Brain Barrier Opening. Current Pharmaceutical Biotechnology, 13(7), 1332-1345.
39. Sun, T., Samiotaki, G., Wang, S., Acosta, C., Chen, C. C., & Konofagou, E. E. (2015). Acoustic cavitation-based monitoring of the reversibility and permeability of ultrasound-induced blood-brain barrier opening. Physics in Medicine and Biology, 60(23), 9079-9094. https://doi.org/10.1088/0031-9155/60/23/9079
40. Attard, P. (2014). The stability of nanobubbles. The European Physical Journal Special Topics, 223(5), 893-914. https://doi.org/10.1140/epjst/e2013-01817-0
41. de Leon, Al & Perera, Reshani & Hernandez, Christopher & Cooley, Michaela & Jung, Olive & Jaganathan, Selva & Abenojar, Eric & Fishbein, Grace & Sojahrood, Amin & Emerson, Corey & Stewart, Phoebe & Kolios, Michael & Exner, Agata. (2019). Contrast Enhanced Ultrasound Imaging by Nature-Inspired Ultrastable Echogenic Nanobubbles. Nanoscale. 11. 10.1039/C9NR04828F.
42. Batchelor, D. V. B., Armistead, F. J., Ingram, N., Peyman, S. A., Mclaughlan, J. R., Coletta, P. L., & Evans, S. D. (2021). Nanobubbles for therapeutic delivery: Production, stability and current prospects. Current Opinion in Colloid & Interface Science, 54, 101456. https://doi.org/10.1016/j.cocis.2021.101456
43. Toepke, M. W., & Beebe, D. J. (2006). PDMS absorption of small molecules and consequences in microfluidic applications. Lab on a Chip, 6(12), 1484-1486. https://doi.org/10.1039/b612140c
44. Dong, X. (2018). Current Strategies for Brain Drug Delivery. Theranostics, 8(6), 1481-1493. https://doi.org/10.7150/thno.21254
45. Leskinen J J, Hynynen K. STUDY OF FACTORS AFFECTING THE MAGNITUDE AND NATURE OF ULTRASOUND EXPOSURE WITH IN VITRO SET-UPS. Ultrasound in Medicine and Biology. 2012; 38(5): 777-94. doi: 10.1016/j.ultrasmedbio.2012.01.019. PubMed PMID: WOS:000302577900007.
46. Leenhardt R, Camus M, Mestas J L, Jeljeli M, Abou Ali E, Chouzenoux S, Bordacahar B, Nicco C, Batteux F, Lafon C, Prat F. Ultrasound-induced Cavitation enhances the efficacy of Chemotherapy in a 3D Model of Pancreatic Ductal Adenocarcinoma with its microenvironment. Scientific Reports. 2019; 9:9. doi: 10.1038/s41598-019-55388-0. PubMed PMID: WOS:000503031800003.
47. Cafarelli A, Verbeni A, Poliziani A, Dario P, Menciassi A, Ricotti L. Tuning acoustic and mechanical properties of materials for ultrasound phantoms and smart substrates for cell cultures. Acta Biomaterialia. 2017; 49:368-78. doi: 10.1016/j.actbio.2016.11.049. PubMed PMID: WOS:000394062100029.

### Example 2: Organ-On-Chip Genomic Based Modeling of BBB Behavior Under Focused Ultrasound

A major hindrance to advances in the care of patients with malignant gliomas is the presence of the blood brain barrier (BBB) and blood-brain tumor barrier (BBTB) that greatly restricts drug access from the plasma to the tumor cells. We develop and test a personalized drug-agnostic approach to treatment facilitated by the transient and repairable opening of the BBB and BBTB, adapted to the genomics of the individual patient.

Bubble-assisted Focused Ultrasound (BAFUS) has proven effective in opening the BBB for treatment of glial tumors in adults and pediatric cases. BAFUS has been previously shown to disrupt noninvasively, selectively, and transiently the BBB in small animals in vivo. However, there is a lack of an in vitro preclinical model suitable for testing the genetic determinants of endothelial cell tight junction integrity and vulnerability to the physical disruption. Our BBB organ-on-chip enables translation of precision medicine of brain cancers by informing patient-specific parameters by which to open the BBB allowing use of drugs and drug combinations otherwise unsuitable. This overcomes one of the most prevalent limitations on drug treatment of brain cancers.

Dynamic remodeling of the BBB occurs in health and disease. Recent studies of alleles or single nucleotide variants of tight junction genes may participate in risk, or progression, of disease. A variant in the 3′-untranslated region of the CLDN5 locus, rs10314, is associated with increased risk of schizophrenia in Chinese Han population and an Iranian population. The rs10314 variant was associated with diminished expression of Claudin-5 in endothelial cells of the BBB. This risk of schizophrenia from the rs10314 variant is higher in females than males. Our initial focus on alleles and SNVs of Claudin-5 (among the nine genes coding for BBB tight junctions) stems from the implication of aberrant allele-determined expression, as well as the demonstrated suppression of Claudin-5 expression by inflammatory cytokines (IL-6) and stimulated through serotonin/5-HT1A receptors.

Develop and characterize BAFUS BBB opening parameters for organ-on-chip platform.

We first develop and test BAFUS physical parameters on bubble cavitation inside the device. In an embodiment, FUS generates oscillating pressure in water that causes cavitation of bubbles within its field. Peak-negative Pressure (PNP) is usually used for cavitation characterization. There are mainly two types of cavitation (5): stable cavitation, where bubbles oscillate in size without rupture, and inertial cavitation, where the bubbles are ruptured by excessive pressure. Both cavitation modes can open the BBB, but inertial cavitation tends to cause tissue damage. There are also two types of bubbles used for BBB disruption, microbubbles (a few micron in size) (6) and nanobubbles (100-900 nm) (7). Both bubbles can open the BBB. Commercial bubbles are expensive. Many self-made bubbles were also reported (8)(9)(7), which can be much cheaper. In an embodiment, to facilitate the extensive BAFUS parameter optimization, we use home-made nanobubbles, as demonstrated by our results.

In an embodiment, to test bubble cavitation inside the device, the bubble solution is perfused through one channel of the device with the other channel filled by PBS and sealed. Then 1 MHz burst FUS signals are applied (Low frequencies ~0.2 to 1.5 MHz are usually used for BAFUS treatment due to large signal attenuation by the skull at higher frequencies). The cavitation is monitored by the sub-(0.5f0) and superharmonic (1.5f0, 2.5f0 . . . ) peaks in frequency domain, which are unique to bubble cavitation (10). Both types of peaks have been used for cavitation monitoring (9)(11). We use subharmonic. The height of the peak indicates the magnitude of the cavitation. The broadening of the peak is an indication of the inertial cavitation (9). The cavitation signals are characterized for different BAFUS parameters listed below to identify different conditions of stable cavitation and onsets of inertial cavitation. Based on literature of both in vivo experiments and clinical trials (7)(12)(13)(14), we identify baseline conditions for 5 BAFUS barrier opening physical parameters that we test:

1) Burst condition: usually multiple burst signals are used for BAFUS treatment. We test burst conditions from the literature, i.e. ~30-90 burst signals per treatment with a burst repetition rate of ~1 Hz (1 sec/burst), and burst duty cycle ~0.1-1% (1-10 ms FUS signal per burst).

2) PNP at the FUS focal point: PNP for BAFUS BBB disruption was reported at ~0.5 MPa (13). PNP for inertial cavitation was reported at ~0.91 MPa (9). We plan to test PNP between 0.1 to 1.2 MPa.

3) Bubbles: Two home-made nanobubbles are tested (see Results).

4) Bubble dilution factor: up to 20 μl/kg bubble solution volume per body weight were reported in clinical trials (13), indicating a dilution factor of 3,500× (considering adult blood volume of 70 ml/kg). Dilution factors up to 100× were also reported for nanobubbles in vivo (7). We test dilution factors 10 to 5000 to find out how the cavitation signal changes with dilution factor.

5) Bubble flow rate: Flow rate effect has not been previously reported due to the lack of the in vitro models for BAFUS barrier opening. How cavitation signal changes with flow rate is studied. One embodiment has a channel volume of 88 μl. We test flow rate around 88 μl/sec, which replaces the whole channel volume for each burst signal.

Fabrication of organ-on-chip device with US-transparent window: In an embodiment, a novel organ-on-chip device with an US-transparent window using thin membranes (15)(16) is fabricated to avoid unintended US “hot spots” (energy uncertainty up to 700% (17)) in traditional cell culture setup. The device comprises 4 polydimethylsiloxane (PDMS) frame layers (1 mm thick) sandwiching 2 PDMS liquid confining membranes (20 um thick; top and bottom) and 1 clear porous Polyethylene Terephthalate (PET) membrane (0.4 um pore, 11 um thick; middle) (FIG. 8A). The device is bonded using uncured PDMS as an adhesive to prevent leaks (18). FIG. 9 shows an exemplary embodiment of a fabricated device filled with colored dyes. FIG. 8B shows an exemplary embodiment of a 3D printed world-to-chip interface to allow tubing connection through side fittings for better reliability and without interference with the US signal. The center US-transparent window is ~6 mm diameter.

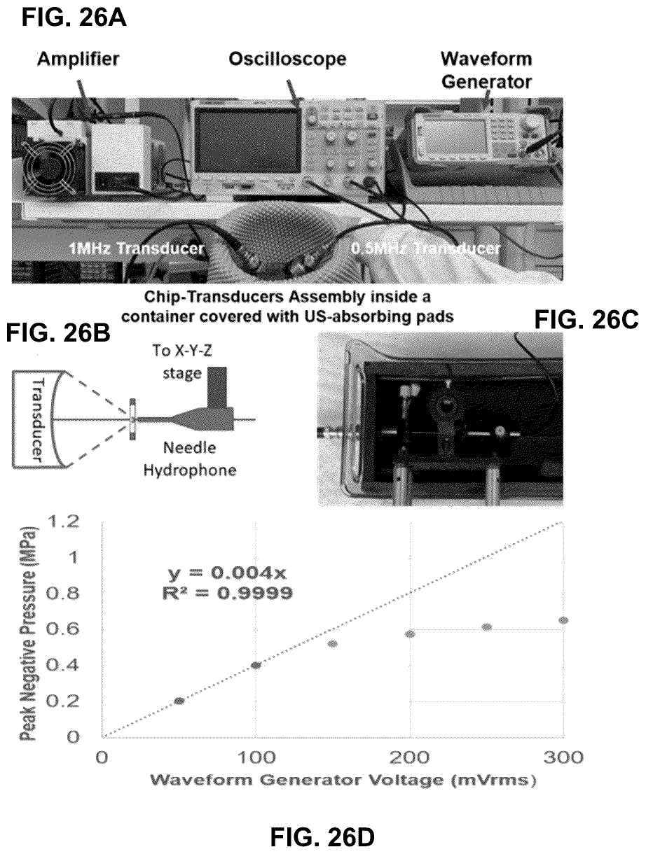

FUS system and US-transparent window, PNP characterization: In an embodiment, a FUS system for BAFUS treatment is shown (FIG. 26A). In an embodiment, a waveform generator generates the burst signal that is amplified and sent to a 1 MHz focused US transducer. A 0.5 MHz focused US transducer is used for cavitation monitoring. The cavitation signal is recorded by a digital storage oscilloscope and displayed in frequency domain by Fast Fourier Transform (FFT). A 3D-printed holder is used to form a chip-transducers assembly so that the focal points of the transducers fall within the US-transparent window of the device. The assembly is housed inside a container covered with US-absorbing pads to reduce noise. During experiments, the container is filled with deionized (DI) water degassed by vacuum in a desiccator to an oxygen level <1 ppm. A 1 mm needle hydrophone (Precision Acoustics, UK) mounted on a X-Y-Z stage is used to measure the PNP at the focal point of the 1 MHz transducer and characterize the US-transparent window of the chip (FIG. 26B). With an input voltage Vin of 100 m Vrms, PNP is measured to be 0.40 MPa, either directly or with the US-transparent window of the device between the transducer and the needle hydrophone. This confirms that the US-transparent window generates negligible losses, and accurate US energy can be applied to the bubbles inside the chip window area. PNP vs. Vin is also measured, which shows saturation of PNP at 0.65 MPa due to the transducer impedance not matched to 50 ohm (reflection and forward voltage ratio was measured to be 0.82, implying 0.82\hat{ }2-67% power reflected).

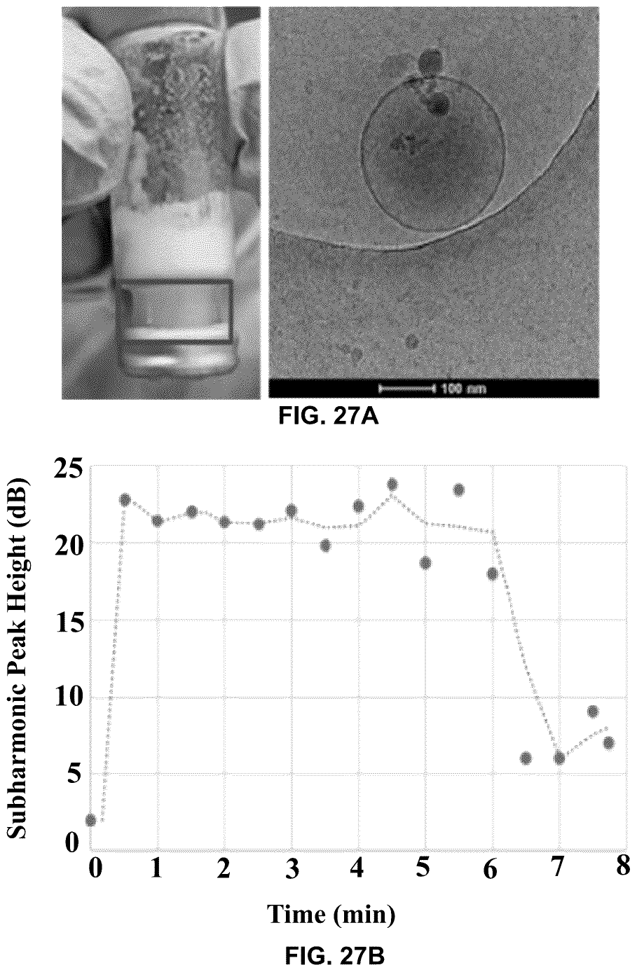

Nanobubble fabrication and characterization: In an exemplary embodiment, two nanobubbles from the literature are fabricated, i.e. a propylene glycol-glycerol (PG-Gly) recipe (19) and a Pluronic L10 recipe (7). Briefly, mixture of lipids DBPC:DPPA:DPPE:mPEG-DSPE=6:1:2:1 is dissolved in PBS containing either 5% PG+5% Gly, or 0.6 mg/ml L10+5% Gly. 0.5 ml solution is then transferred to a 2.5 ml glass vial and sealed. The air inside the vial is then replaced by C3F8 gas. The bubbles are activated by an amalgamator and centrifuged at 50 g for 5 min, then ~250 ul nanobubble solution is extracted (FIG. 27A). The diluted bubbles are then flown in the top channel of the chip with PBS filling the bottom channel. Under Vin=100 m Vrms, both recipes show bubble cavitation (manifested by the sub/superharmonic peaks). On the other hand, the control PBS solution shows only the fundamental and harmonic peaks (f0, 2f0, 3f0 . . . ) (FIGS. 27B, 27D). FIG. 27C also shows that the cavitation signal for a 100× diluted PG-Gly sample lasts the duration of the bubble flow inside the channel (~5.5 min with a flow 1 ml/min), which is much longer than the 30-90 sec for BAFUS treatment. Finally, Cryo-TEM shows our nanobubbles are similar to that reported in literature (~200 nm with haze center, FIG. 27A) (19).

Demonstrate on-chip barrier permeability measurements and BAFUS barrier opening using long term brain microvascular endothelial cell (BMVEC) line (HBEC-5i): Two popular measurements of the barrier property are TransEndothelial Electrical Resistance (TEER) and permeability (Pe) (20). TEER measurement can be done in transwell format easily. TEER measurement in chips is more complicated (21). In some embodiments, Pe measurement is taken on-chip. TEER and Pe in corresponding transwell with similar PET membranes is used as a control.

In an embodiment, TEER measurement in transwell is done using an EVOM2 Epithelial Voltohmmeter (WPI Inc., FL) with a STX2 electrode set. Pe measurements in both transwell and chip are done similar to transwell Pe measurement reported in literature (22)(23). Briefly, the top well/channel is loaded with fluorescent labeled tracer with concentration C0. After certain time interval Δt, the fluid at the bottom well/channel is carefully extracted with a volume V. The concentration of the extracted tracers Ct is measured by a BMG Clariostar plate reader with fluorescence capability. Pe can be expressed (Fick's Law) as:

$$P e = \frac{J}{Δ C} = \frac{\frac{C t * V}{Δ t} / A}{Δ C} \approx \frac{C t * V}{C 0 * Δ t * A} = \frac{C t * V}{C 0 * Δ t * A 0 / cos ( θ )} \qquad ( EQ13 )$$

where ΔC is approximated by C0 if Ct<<C0, A is the permeable area. For BAFUS barrier disruption, A=A0/cos(θ), where A0 is the transverse area of the focused beam, and θ is the incident angle. If necessary, on chip stable cell-electrode interface are optimized using microelectrode configurations (ScioSpec, Germany).

In an embodiment, long term BMVEC line (HBEC-5i, PI Berens) is used to form cell barrier in the chip device for BAFUS barrier disruption testing. Cells are seeded to the top channel and perfused by a peristaltic pump inside a CO2 incubator; the bottom channel is filled with culture medium and sealed (perfusion can also be done if needed). Currently, physiological wall shear stress (WSS) is not applied to the cell layer, but can be if needed. The duration of the healthy status of the confluent cell layer without BAFUS disruption is monitored by the change of Pₑ over time Pₑ(t). Different tracers are used for Pₑ measurement. One example is Lucifer Yellow, which has a molecular weight (444 Da) similar to common chemotherapy drugs. Another example is 150 KD Dextran conjugated with fluorescent dye, approximately the size of antibodies. Pₑ and TEER measurements of the cell barrier in transwell control over time are used for comparison.

In an embodiment, with a confluent cell layer inside the chip, BAFUS is performed to disrupt its barrier with the device in 37° C. degassed DI water to reduce thermal shock effect. The change of permeability over time ΔPₑ(t)=Pₑ(t)−P_{e,0} is measured, where P_{e,0} is the permeability before disruption. The recovery time is defined as the time for ΔPₑ(t) to drop to a certain percentage of ΔPₑ(0) (e.g. 10%). Literature has suggested a barrier open duration (i.e. the recovery time) of a few hours to within 24 hours (25). For example, 0.5, 1, 4 and 24 hours time periods are tested. Due to the time scale of the recovery, Pₑ measurement needs to be done quickly. A Δt of ~5 min is used for Pₑ measurement. Tracer size, initial concentration are tested to get a measurable signal at the bottom channel of the device. The ΔPₑ(0) and recovery time are also studied for different BAFUS settings, such as PNP, bubble dilution factor, burst conditions. Live/dead staining is used to check the damage of the cells under different conditions to identify parameter thresholds for cell damage. Phase contrast images of cells before and after the disruption are also used to identify physically damaged cells.

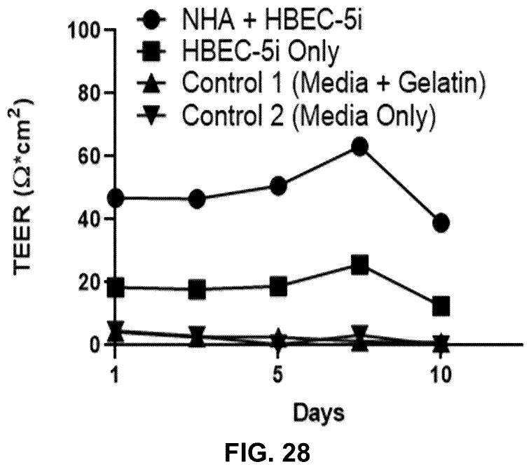

In an embodiment, HBEC-5i cells are cultured on transwell insert with 0.4 um PET membrane. 105 k/cm² cell seeding density has been identified to form a confluent cell layer in 24 hours. The TEER measurement show results consistent with literature, and the high resistant state lasts for ~7 days (FIG. 28), long enough for barrier disruption and recovery study.

In an embodiment, a confluent layer of HBEC-5i will be cultured inside the chip device, similar to that for transwell insert. HBEC-5i layer is much leakier than the real BBB. However, a measurable statistically significant permeability difference before and after the BAFUS disruption is observable by selecting tracer size and initial concentration. Other common cells such as HUVEC can also be used if needed. Higher power BAFUS settings generate a higher ΔPₑ(0) and longer recovery time. Detrimental BAFUS settings are those with significant cell death and a much longer recovery time. Currently, the cell barrier disruption and repair mechanisms can involve both cell-cell junction disruption and cell damage. A mannitol disruption model recently showed intact cell tight junctions and focal leaks during disruption (26). The main focus is on the permeability and dead/live staining in an embodiment. But junction protein fluorescent staining and in situ imaging of the disrupted barrier, similarly to those in the mannitol disruption model, could also be used if needed.

Measurement of barrier performance of iPSC-derived BBB in transwell and as organ-on-chip, including: i) cell-cell TJ integrity by TEER for iPSC-derived endothelial cells, ii) cell-cell TJ integrity by TEER and permeability measurements for iPSC-derived endothelial, pericyte, and astrocyte multi-cell mixture BBB models, and iii) profile the allele variances of 9 TJ genes in six iPSC lines (3 female, 3 male).

In an embodiment, development of in vitro models of BBB (27) enable studies of the biology, physiology, and pathophysiology of this crucial micro-anatomical feature (28). Brain microvascular endothelial cells (BMVECs) along with cognate astrocytes, pericytes, and neurons are each derived from iPSCs (29)(30)(31). iPSC-derived BMVEC and other BBB cell types have yet to be applied to the study of barrier performance based on the interplay of alleles among the 9 different genes which form the TJs or to probe genetically-determined BBB variability to BAFUS disruption.

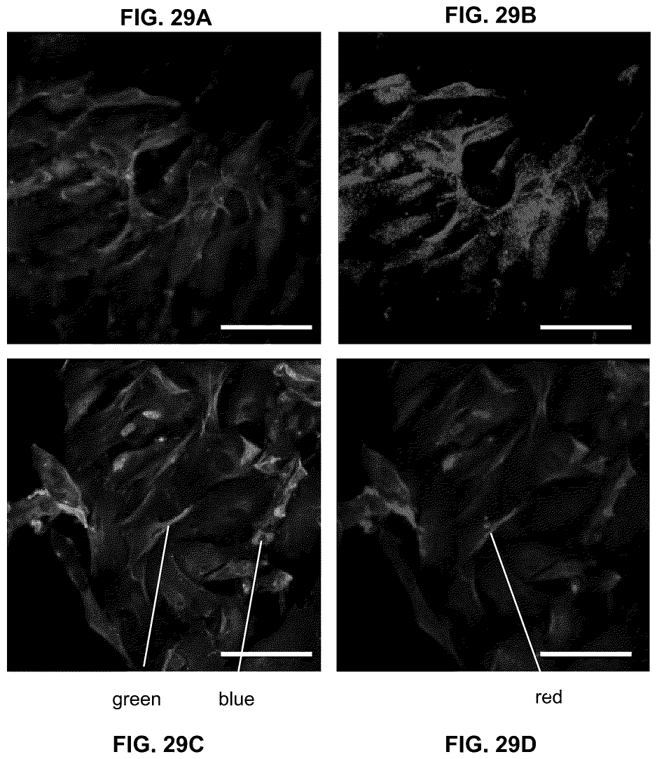

iPSC differentiation to BMVEC and Immunofluorescence for TJs.: Human iPSCs differentiate into BMVEC at 5% O₂ conditions (32). Briefly, iPSCs seeded on 6-well plates coated with Matrigel (33) expand to a mixed endothelial cell and neural progenitor cell culture by switching cells to unconditioned medium (UM) for 6 days. Endothelial cells selectively expand by switching to endothelial cell (EC) media supplemented with retinoic acid (RA) at 5% O₂. In an embodiment, first passage iPSC-derived BMVECs from Model 1 and Model 2 are grown on gelatin-coated Transwells to 100% confluence, then fixed and stained with primary antibodies rabbit anti-TJP1 (HPA001636) or mouse anti-CD31 (MMS-484R), then incubated with appropriate secondary antibodies (FIG. 29).

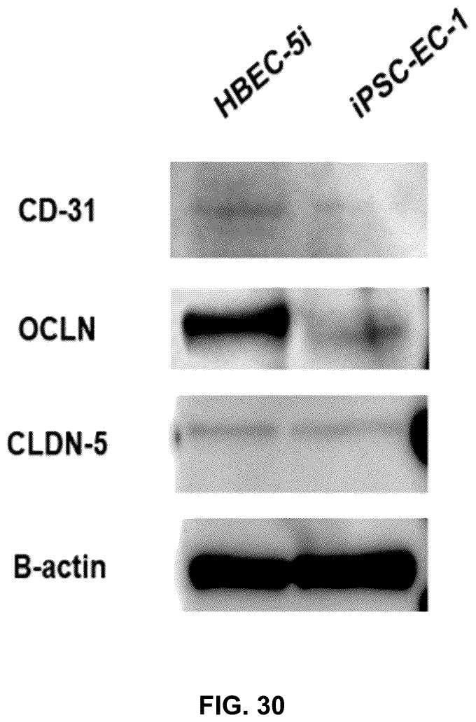

Western blot for TJ proteins: In an embodiment, lysates (15 μg protein) collected from human endothelial cells (HBEC-51; ATCC) and iPSC-derived BMVECs Model 1 are electrophoresed, blotted and probed for endothelial cell CD-31 (MMS-484R) and TJ markers: occludin (HPA005933), claudin-5 (ABT45). β-actin (Cell Signaling #4967) are loading control (FIG. 30). Densitometry analysis (ImageJ) reveals occludin and CD-31 expression is ~50% and claudin-5 is 75% of that of HBEC-5i endothelial cell line (normalized to loading control).

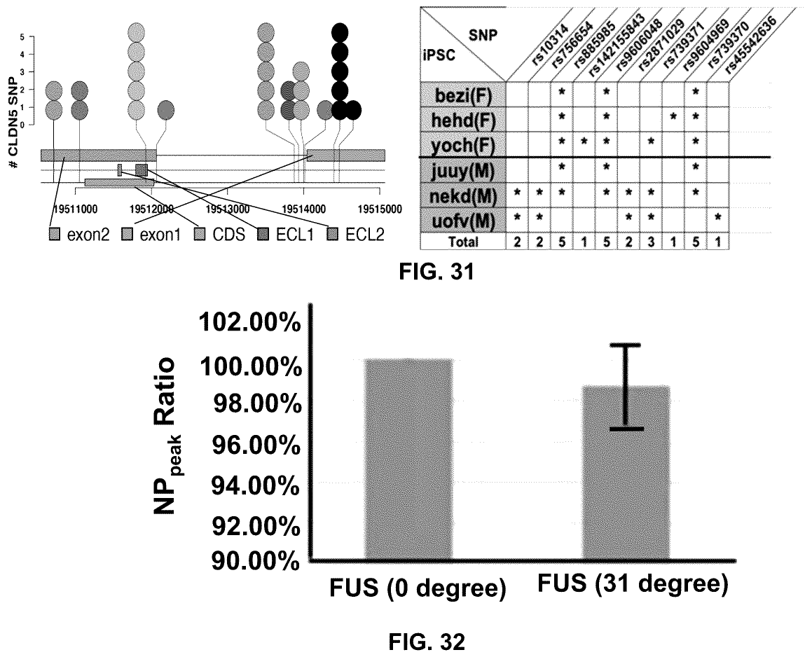

Selection of iPSC lines: In an embodiment, from the HipSci Consortium (34), iPSC lines are chosen based on derivation from non-diseased subject, age range 65-69, high diversity of progeny, 3 female and 3 male, and comprehensive existing, accessible molecular data. These 6 lines harbor single nucleotide variants (SNVs) in the claudin-5 gene (CLDN5) (FIG. 31). CLDN5 provides one of the most abundant and structurally significant features of the TJ in the BBB. These 6 models afford findings that generate hypotheses on TJ alleles and BBB performance (genotype/phenotype).

TEER measurement in co-culture: In an embodiment, co-cultured BBB cells form durable barriers with physiological TEER values. HBEC-5i (ATCC) in monolayer growth forms TJs that strengthen over time (FIG. 28). Coculture of NHA with HBEC-5i establishes a multicellular barrier with higher TEER and progressively stronger junctions over 7 days.

In differentiated iPSC-derived BMVECs, measure cell-cell TJ integrity by TEER and Pₑ.: iPSC-derived BMCECs display physiologically relevant characteristics of the blood brain barrier, primarily TEER and TJ integrity.

Standardized protocols guide the production of BMVECs manifesting endothelial markers and function (OCLN, ABCB1, SLC2A1, TJP1). In an embodiment, iPSC-derived BMVECs are seeded in transwells and device (6 replicates) equipped for TEER and Pₑ measurements (as in Aim 1) and followed for durability of patency (high levels of resistance) over multiple days. Confluent monolayers of iPSC-derived BMVECs are fixed and stained using antibodies for TJ Protein 1 (TJP1) and CD-31. Total protein lysates are collected and analyzed by western blot: occludin (OCLN), claudin-5 (CLDN-5), and CD-31. Serial TEER and Pe measurements on six different iPSC-derived BMVECs (3 female, 3 male) are made over 1 week. Pₑ from both transwell and device are compared.

Differentiation of iPSC to BMVEC is a well-established technique with high likelihood of success with rigorous metrics of TJ protein expression. Performance of the selected specific iPSCs to perform as BBB is unknown. Each iPSC yields BMVECs suitable for confluent monolayer formation. Patency of each of the BBBs differs. iPSC-derived BMVECs that are least similar to each other become the premise for hypothesis development based on TJ gene allelotype or SNVs. Age of the iPSC donor may prove to be a determinant of differentiation to BMVEC.

Measure cell-cell TJ integrity by TEER and Pₑ for iPSC-derived endothelial, pericyte, and astrocyte multi-cell mixture BBB models: Once iPSCs are differentiated as BMVECs, co-culture with astrocytes and pericytes strengthens barrier integrity and TEER (36).

In an embodiment, iPSC-derived BMVECs are numerically expanded, differentiated into astrocytes and pericytes using published methods (30). Cell type identity is confirmed by qRT-PCR of marker genes, Western blot and immunofluorescence of marker proteins. Inclusion of astrocytes and pericytes, and culture under fluid flow dynamics are each reported to elicit barriers with TEER properties approaching in vivo conditions (37); we use initial mixing ratios of BMVEC:Astrocyte:Pericyte of 4:2:1. TEER and Pe are measured as in Aim 1 and Pe compared between transwell and device.

TEER values of co-cultured commercial ECs mixed with astrocytes without pericytes and dynamic fluid flow are likely to be in the range of 100 Ohms cm-2 (38), but up to 4,000 Ohms cm-2 with iPSC-derived BMVEC, astrocytes, pericytes, and neurons (39). Similarly, expression of key TJ proteins in iPSC-derived BMVEC is enhanced by the co-culture of BBB cell types. Across the 6 different iPSC-derived BMVEC monolayer cultures we track cell passage number, culture media, and cell culture duration (40). TEER values are monitored and compared. Optimal mixed cell type proportions are not the same for each iPSC BBB's peak TEER value; this sets the stage for hypothesis development, and specific testing for the influence of TJ gene alleles or SNVs on BBB integrity.

Profile the allele variances and single nucleotide variances of 9 TJ genes in six iPSC lines (3 F, 3 M): In an embodiment, allelic variants of 9 TJ genes in the 6 iPSC lines are queried for possible association with susceptibility to transient opening of the BBB using BAFUS. SNPs previously determined to have significant consequences and/or are within 1 kb of SNPs associated with neurological disorders in these 9 genes are identified using the variant effect predictor tool (41)(42). Allelic variants at UTRs may affect the overall function, transcription, secondary structure, stability, and interaction with regulators which could modify molecular processes and molecular pathways (43).

For example, in an embodiment, the rs730882227 SNP in the OCLN gene introduces a frameshift and has been associated with neurological disorders (44). The rs115253607 (OCLN) SNP resides in the 5′ UTR region proximal to the transcriptional start codon which serves as a region of interest to examine for promoter involvement. TJ genotype of BBB confers vulnerability to treatment with BAFUS. Because BAFUS has been shown to transiently disrupt TJ complexes to facilitate transcapillary molecule movement, allelic variants of these complexes can help group parameters for a noninvasive opening.

Testing the vulnerability of iPSC-derived BMVEC monolayers and co-culture models to disruption by BAFUS, measuring variance in disruption (Pe) and recovery over time: In an embodiment, two elements of therapeutically-intended BBB disruption are: i) extent of disruption and ii) degree of recovery from disruption. Clinically-relevant disruption by BAFUS is envisioned as opening the permeability in a manner that allows molecules (drugs or antibodies) passage across the tight junction (sensitive to molecular weight) while avoiding lethal damage to the BMVEC so that recovery is feasible. A contribution to an embodiment is the development of metrics of BAFUS disruption and recovery features.

In an embodiment, we measure the permeability change after barrier disruption ΔPe(t) as in Aim 1 for all iPSC lines, as well as their co-culture barriers. Besides ΔPe(t), we further define the BAFUS opening relative to the opening induced by exposure to ~1.4 M mannitol for 5 min, and Repair defined as the degree to which pre-treatment values of Pe are recovered at certain time t, i.e.:

$$Opening = Pe , BAFUS ( 0 ) / Δ Pe , mannitol ( 0 ) * 100 \% ; \qquad ( EQ14 )$$

$$Repair = [ 1 - Δ Pe ( t ) / Δ Pe ( 0 ) ] * 100 \%$$

Table 5 illustrates the barrier opening and repair for different iPSC lines and tracers under different BAFUS conditions. For example, we vary PNP (from ~0.1 to 1.2 MPa) and the number of bursts N (from 1 to 100) of BAFUS to transiently open the tight junctions of the BBB that leads to Lucifer Yellow Pe values at 35, 65, and 90% of a reference mannitol opening value (BAFUS conditions A, B and C for iPSC #2). The permeability of 150 KD Dextran tracer is measured at the same time. To evaluate repair, the ΔPe(t) to both tracers is measured along a 24-hour time course, e.g. after cell recovery inside a CO2 incubator for 0.5, 1, 4 and 24 hours. The opening and repair values are compared to identify statistically significant differences between the lines.

In an embodiment, we select conditions that promote more effective opening and better recovery. For recovery, different time scales have different repair mechanisms, such as gene expression, translation, posttranslational modifications, etc. Literature has suggested 4-20% variation in intact permeability values of in vitro BBB models with no significant difference between models from healthy individuals. Variation increases to 38% for mannitol disrupted permeability with no comparison between models. We study the possible difference in BAFUS disruption of BBB among different iPSC lines.

### References for Example 2

1. Beccaria K, Canney M, Bouchoux G, Puget S, Grill J, Carpentier A. Blood-brain barrier disruption with low-intensity pulsed ultrasound for the treatment of pediatric brain tumors: A review and perspectives. Neurosurg Focus. 2020; 48(1).
2. Idbaih A, Canney M, Belin L, Desseaux C, Vignot A, Bouchoux G, et al. Safety and feasibility of repeated and transient blood-brain barrier disruption by pulsed ultrasound in patients with recurrent glioblastoma. Clin Cancer Res. 2019; 25(13).
3. Zhang D Y, Dmello C, Chen L, Arrieta V A, Gonzalez-Buendia E, Robert Kane J, et al. Ultrasound-mediated delivery of paclitaxel for glioma: A comparative study of distribution, toxicity, and efficacy of albumin-bound versus cremophor formulations. Clin Cancer Res. 2020; 26(2).
4. Berndt P, Winkler L, Cording J, Breitkreuz-Korff O, Rex A, Dithmer S, et al. Tight junction proteins at the blood-brain barrier: far more than claudin-5. Cell Mol Life Sci. 2019; 76(10).
5. Bader K B, Holland C K. Gauging the likelihood of stable cavitation from ultrasound contrast agents. Phys Med Biol. 2013; 58(1).
6. Shi L, Palacio-Mancheno P, Badami J, Shin D W i., Zeng M, Cardoso L, et al. Quantification of transient increase of the blood-brain barrier permeability to macromolecules by optimized focused ultrasound combined with microbubbles. Int J Nanomedicine. 2014; 9.
7. Cheng B, Bing C, Xi Y, Shah B, Exner A A, Chopra R. Influence of Nanobubble Concentration on Blood-Brain Barrier Opening Using Focused Ultrasound Under Real-Time Acoustic Feedback Control. Ultrasound Med Biol. 2019; 45(8).
8. Pouliopoulos A N, Jimenez D A, Frank A, Robertson A, Zhang L, Kline-Schoder A R, et al. Temporal Stability of Lipid-Shelled Microbubbles During Acoustically-Mediated Blood-Brain Barrier Opening. Front Phys. 2020; 8.
9. Wu S K, Chu P C, Chai W Y, Kang S T, Tsai C H, Fan C H, et al. Characterization of different microbubbles in assisting focused ultrasound-induced blood-brain barrier opening. Sci Rep. 2017; 7.
10. Lauterborn W. Numerical investigation of nonlinear oscillations of gas bubbles in liquids. J Acoust Soc Am. 1976; 59(2).
11. Tsai C H, Zhang J W, Liao Y Y, Liu H L. Real-time monitoring of focused ultrasound blood-brain barrier opening via subharmonic acoustic emission detection: Implementation of confocal dual-frequency piezoelectric transducers. Phys Med Biol. 2016; 61(7).
12. Pouliopoulos A N, Wu S Y, Burgess M T, Karakatsani M E, Kamimura HAS, Konofagou EE. A Clinical System for Non-invasive Blood-Brain Barrier Opening Using a Neuronavigation-Guided Single-Element Focused Ultrasound Transducer. Ultrasound Med Biol. 2020; 46(1).
13. Abrahao A, Meng Y, Llinas M, Huang Y, Hamani C, Mainprize T, et al. First-in-human trial of blood-brain barrier opening in amyotrophic lateral sclerosis using MR-guided focused ultrasound. Nat Commun. 2019; 10(1).
14. Gasca-Salas C, Fernández-Rodríguez B, Pineda-Pardo J A, Rodríguez-Rojas R, Obeso I, Hernández-Fernández F, et al. Blood-brain barrier opening with focused ultrasound in Parkinson's disease dementia. Available from: https://doi.org/10.1038/s41467-021-21022-9
15. Cafarelli A, Verbeni A, Poliziani A, Dario P, Menciassi A, Ricotti L. Tuning acoustic and mechanical properties of materials for ultrasound phantoms and smart substrates for cell cultures. Acta Biomater. 2017; 49.
16. Brekhovskikh L M, Godin O a. Acoustics of layered media I: plane and quasi-plane waves. Plane-Wave Reflection From Boundaries of Solids. 1990.
17. Leskinen J J, Hynynen K. Study of Factors Affecting the Magnitude and Nature of Ultrasound Exposure with In Vitro Set-Ups. Ultrasound Med Biol. 2012; 38(5).
18. Eddings M A, Johnson M A, Gale B K. Determining the optimal PDMS-PDMS bonding technique for microfluidic devices. J Micromechanics Microengineering. 2008; 18(6).
19. De Leon A, Perera R, Hernandez C, Cooley M, Jung O, Jeganathan S, et al. Contrast enhanced ultrasound imaging by nature-inspired ultrastable echogenic nanobubbles. Nanoscale. 2019; 11(33).
20. Destefano J G, Jamieson J J, Linville R M, Searson P C. Benchmarking in vitro tissue-engineered blood-brain barrier models. Fluids Barriers CNS. 2018; 15(1).
21. Wegener J, Seebach J. Experimental tools to monitor the dynamics of endothelial barrier function: A survey of in vitro approaches. Vol. 355, Cell and Tissue Research. 2014.
22. Nooteboom A, Hendriks T, Ottehöller I, Van Der Linden CJ. Permeability characteristics of human endothelial monolayers seeded on different extracellular matrix proteins. Mediators Inflamm. 2000; 9(5).
23. Albelda S M, Sampson P M, Haselton F R, McNiff J M, Mueller S N, Williams S K, et al. Permeability characteristics of cultured endothelial cell monolayers. J Appl Physiol. 1988; 64(1).
24. Shah P, Fritz J V., Glaab E, Desai M S, Greenhalgh K, Frachet A, et al. A microfluidics-based in vitro model of the gastrointestinal human-microbe interface. Nat Commun. 2016; 7.
25. Lipsman N, Meng Y, Bethune A J, Huang Y, Lam B, Masellis M, et al. Blood-brain barrier opening in Alzheimer's disease using MR-guided focused ultrasound. Nat Commun. 2018; 9(1).
26. Linville R M, DeStefano J G, Sklar M B, Chu C, Walczak P, Searson PC. Modeling hyperosmotic blood-brain barrier opening within human tissue-engineered in vitro brain microvessels. J Cereb Blood Flow Metab. 2020; 40(7).
27. Weiss N, Miller F, Cazaubon S, Couraud P O. The blood-brain barrier in brain homeostasis and neurological diseases. Vol. 1788, Biochimica et Biophysica Acta-Biomembranes. 2009.
28. Zhao Z, Nelson A R, Betsholtz C, Zlokovic B V. Establishment and Dysfunction of the Blood-Brain Barrier. Vol. 163, Cell. 2015.
29. Canfield S G, Stebbins M J, Morales B S, Asai S W, Vatine G D, Svendsen C N, et al. An isogenic blood-brain barrier model comprising brain endothelial cells, astrocytes, and neurons derived from human induced pluripotent stem cells. J Neurochem. 2017; 140(6).
30. Lippmann E S, Azarin S M, Kay J E, Nessler R A, Wilson H K, Al-Ahmad A, et al. Derivation of blood-brain barrier endothelial cells from human pluripotent stem cells. Nat Biotechnol. 2012; 30(8).
31. Stebbins M J, Wilson H K, Canfield S G, Qian T, Palecek S P, Shusta E V. Differentiation and characterization of human pluripotent stem cell-derived brain microvascular endothelial cells. Methods. 2016; 101.
32. Park T E, Mustafaoglu N, Herland A, Hasselkus R, Mannix R, FitzGerald E A, et al. Hypoxia-enhanced Blood-Brain Barrier Chip recapitulates human barrier function and shuttling of drugs and antibodies. Nat Commun. 2019; 10(1).
33. O'Shea O, Steeg R, Chapman C, Mackintosh P, Stacey GN. Development and implementation of large-scale quality control for the European bank for induced Pluripotent Stem Cells. Stem Cell Res. 2020; 45.
34. Streeter I, Harrison P W, Faulconbridge A, Consortium T H S, Flicek P, Parkinson H, et al. The human-induced pluripotent stem cell initiative-Data resources for cellular genetics. Nucleic Acids Res. 2017; 45(D1).
35. Neal E H, Marinelli N A, Shi Y, McClatchey P M, Balotin K M, Gullett D R, et al. A Simplified, Fully Defined Differentiation Scheme for Producing Blood-Brain Barrier Endothelial Cells from Human iPSCs. Stem Cell Reports. 2019; 12(6).
36. Linville R M, DeStefano J G, Sklar M B, Xu Z, Farrell A M, Bogorad M I, et al. Human iPSC-derived blood-brain barrier microvessels: validation of barrier function and endothelial cell behavior. Biomaterials. 2019; 190-191.
37. Elbakary B, Badhan R K S. A dynamic perfusion based blood-brain barrier model for cytotoxicity testing and drug permeation. Sci Rep. 2020; 10(1).
38. Daniels B P, Cruz-Orengo L, Pasieka T J, Couraud P O, Romero I A, Weksler B, et al. Immortalized human cerebral microvascular endothelial cells maintain the properties of primary cells in an in vitro model of immune migration across the blood brain barrier. J Neurosci Methods. 2013; 212(1).
39. Lippmann E S, Al-Ahmad A, Azarin S M, Palecek S P, Shusta E V. A retinoic acid-enhanced, multicellular human blood-brain barrier model derived from stem cell sources. Sci Rep. 2014; 4.
40. Srinivasan B, Kolli A R, Esch M B, Abaci H E, Shuler M L, Hickman J J. TEER Measurement Techniques for In Vitro Barrier Model Systems. Vol. 20, Journal of Laboratory Automation. 2015.
41. Naithani S, Geniza M, Jaiswal P. Variant effect prediction analysis using resources available at gramene database. In: Methods in Molecular Biology. 2017.
42. Thormann A, Halachev M, McLaren W, Moore D J, Svinti V, Campbell A, et al. Flexible and scalable diagnostic filtering of genomic variants using G2P with Ensembl VEP. Nat Commun. 2019; 10(1).
43. Zhang X, Wakeling M, Ware J, Whiffin N. Annotating high-impact 5′ untranslated region variants with the UTRannotator. Bioinformatics. 2021; 37(8).
44. Alazami A M, Patel N, Shamseldin H E, Anazi S, Al-Dosari M S, Alzahrani F, et al. Accelerating novel candidate gene discovery in neurogenetic disorders via whole-exome sequencing of prescreened multiplex consanguineous families. Cell Rep. 2015; 10(2).

### Example 3: In-Vitro Research Device to Optimize Bubble-Assisted Focused Ultrasound (BAFUS) for Efficient Drug Delivery to the Brain Using Blood-Brain-Barrier (BBB)-On-Chips

A major obstacle in drug development for neurodegenerative diseases (NDD) is the permeability of the BBB, which restricts systemic drug delivery to diseased brain regions. Today, no FDA-approved tool can bring drugs across the BBB in a non-invasive and drug-agnostic manner. BAFUS is a new technology that temporarily spreads the endothelial vessel walls to open the BBB (BBBO) by vibrating peripherally-injected microbubbles (MBs) using acoustic energy. Recently, improved drug permeability across the BBB has been demonstrated. Although results from BAFUS devices have been successful and generally safe for BBBO, BAFUS dosing has not yet been optimized because no in-vitro model is available for the process. Currently, animal models are the only feasible strategy for examining potential BAFUS optimization, making it costly in both time and resources. Without an in-vitro benchtop tool to relate the dynamics of ultrasound, MB, and BBB permeability, NDD clinical research will continue to be thwarted by BBB biology.

To address this unmet need, we provide an in-vitro modular and ultrasound-transparent BBB-on-a-chip to optimize the effects of BAFUS dosing on human cellular barrier integrity, vulnerability, and repair. In an embodiment, such a benchtop tool can drastically reduce animal testing for NDD by standardizing BAFUS dose through highthroughput parameterization studies. This tool will lead to more efficient BBBO for different drug classes and safer patient ultrasound doses (e.g., shortened sonication time). Our tool uses organ-chips. It differs from other in-vitro BBB models by its ultrasound transparent window combined with microbubble channel for BAFUS treatment. Our treatment uses low-frequency ultrasound (~250 kHz; reducing in-vivo skull attenuation) to vibrate microbubbles flowing adjacent to a cellular monolayer of brain microvascular endothelial cells (BMVECs). The monolayer lines the porous floor of the top flow channel, allowing the bottom channel to measure tracer concentrations across the membrane. We provide a BBB-on-chip sonification tool that optimizes the in vivo effects of BAFUS on the transport of different drugs across the BBB by:

Construct the 250 kHz BAFUS BBB-chip tool and characterize MB stable cavitation parameters. In an embodiment, we integrate a clinically relevant ultrasound transducer and MBs with a BAFUS benchtop setup and use cell-less BBB-chips to establish a working platform of FUS and MBs for subsequent studies. Metric of Success: Conditions for onset of inertial cavitation and maintaining stable cavitation during BAFUS treatment are identified using the new BAFUS BBB-chip setup.

Demonstrate BBBO of Caco-2 BBB-chip in the new BAFUS system. In an embodiment, we construct Caco-2 epithelial cell monolayers as a BBB-on-chip surrogate and measure sensitivity to BAFUS-induced barrier opening. Tracer permeability is measured to compare BAFUS-treated versus non-treated Caco-2 chip samples. Success metrics: At a specific BAFUS dose, statistically significant (p<0.05) increase in measured permeability of BAFUS-treated samples compared to non-treated BBB-on-chips.

Demonstrate iPSC-derived BMVEC barrier formation; Demonstrate barrier opening of iPSC-derived BMVEC (BBB)-on-chip by the BAFUS system. In an embodiment, human iPSC-derived BMVEC barrier are developed according to abundant literatures using a simple fully defined serum-free protocol with repeatable high TEER values (2-8 k ohm-cm2). BAFUS BBBO is tested the same way as in Aim 1B. Success metrics: 2A: Formation of BBB barrier with TEER >1-2 k ohm-cm2 in transwell. 2B: Statistically significant (p<0.05) increase in measured permeability of BAFUS-treated samples compared to non-treated BBB-on-chips.

Establish BAFUS dose curves & onset of BBBO and BBB damage for a large and small tracer. In an embodiment, various ultrasound doses are applied to the cellular barrier, optimizing for 440 Da and 150 kDa tracer permeability (representing small molecule and antibody drug permeability, respectively). BBBO and BBB damage are maximized and minimized, respectively, at the two tracer sizes. Metric of Success: Repeatable dose and recovery curves can be established. This data determines optimized ultrasound dose curves for follow-on in-vivo drug validation studies.

Neurodegenerative diseases (NDD), which include Alzheimer's disease (AD), Parkinson's disease (PD), Multiple sclerosis (MS), and other diseases that comprises an estimated 7 million US patients per year, are difficult to treat due to the inability to deliver clinically efficacious drug concentrations to the affected or diseased portions of the brain.1 Consequently, clinical studies for AD, PD, and MS are proving difficult with very low clinical trial success. Recently, in human clinical studies, the use of an embodiment of bubble-assisted focused ultrasound (BAFUS) demonstrated the ability to disrupt the BBB at plaque sites safely. Animal model data also has demonstrated the value of BAFUS BBB opening (BBBO) in which a 5× increase in the concentration of the anti-amyloid therapeutic molecule, Aduhelm, has been observed.2 The blood-brain barrier (BBB) is our biological defense mechanism protecting the brain from various internal and external threats. It comprises a primary layer of tightly joined endothelial cells and prevents all but essential substances from reaching brain tissue. Consequently, many potentially therapeutic molecules cannot cross from the bloodstream into the target areas in the brain. The challenge of crossing the BBB has led to drug trial failures and is a primary reason we still lack effective treatments for many brain diseases.

BAFUS technology has produced extensive clinical trial data after extensive preclinical research over the past two decades. To date, 62 patients have received 187 BAFUS treatments.3-9 The challenge with BAFUS has been in determining the ideal ultrasound dosage per treatment, as the biology of the BBB has limited in-vitro models, and therefore the cost of preclinical research to investigate ultrasound dosing is steep and carries an enormous burden to animal welfare. While 3D BBB organoids exist,10 their intracellular matrix is too fragile to promote blood vessel formation in-vivo, making microbubble flow in such systems infeasible with current technologies. Traditional top-bottom-compartments BBB-on-chip devices10 do not have the ultrasound-transparent window that prevents hot spot formation with energy uncertainty up to 700%.11 Currently, no BBBchip platforms are designed for quantitative studies of BAFUS BBBO.

BAFUS dosage has been derived through empirical observation rather than experimental reasoning. There is a range of untested dosage parameters that reduce the risk of adverse events and for which permeability across the BBB can be optimized according to therapeutic targets. In an embodiment, our device is a research tool that serves as a low-cost, first-pass solution for pharmaceutical developers to assess the BAFUS BBB permeability of their therapeutic assets. Our BAFUS BBB-chip device de-risks and simplifies future NDD drug development by screening for BBB permeability in-vitro ahead of dose escalation studies, saving animal subjects from use and thereby reducing the time and costs of bringing these drugs to human trials.

BAFUS treatment to open the BBB temporarily, safely, and effectively has been extensively demonstrated in animal model systems, including mice, rats, and NHP. Human Clinical Safety of BAFUS devices: Additionally, albeit utilizing first-generation devices—which are either invasive, require specialized and expensive equipment, and require extensive staff to conduct the procedure—strong safety profiles have been demonstrated. These devices are generally too invasive for high-throughput parameterization studies; one is a surgically implanted array of transducers.12 Another is an MRI-combination device that requires several hours of MRI time and the patient to wear a fixation frame.4 The least invasive first-generation BAFUS device uses optical registration of the patient's facial structure to guide its therapeutic transducers.13

The design of NDD trials has not evolved at the same pace as that for other diseases. We rely primarily on imaging (CT/MR) as a principal measure of outcomes when brain surgery, chemo, or drugs are introduced to a patient's care. BAFUS technology aims to remedy this in an embodiment by providing a non-invasive means of temporarily opening a door into the diseased brain region, allowing for patient selection via diagnostic biomarker assays and complementary pharmacodynamic drug analysis. The lack of an in-vitro model to study BAFUS creates an undue burden on this nascent field, promoting research of more invasive, riskier, and less-informative technologies for NDD research.

An embodiment comprises a noninvasive transcranial dynamic focused ultrasound (TcDFUS) cap and workstation which use proprietary guidance and coupling methods to reach multiple regions of the brain within a half-hour outpatient visit. In an embodiment, we use the in-vitro BAFUS BBB-chip tool to promote drug development and provide clinical services to the biopharmaceutical industry.

Advantages of an embodiment of our BAFUS BBB-chip platform include:

Human-informed: In an embodiment, cell lines from human induced pluripotent stem cells are differentiated into endothelial cells to produce a barrier similar in strength to human models. A standardized growth protocol allows us to generate several chips with the same specifications each week, allowing for high-throughput screening of BAFUS-drug interactions.

Ultrasound Transparency Window: In an embodiment, the geometry of the chip reduces scattering of ultrasound and provides an unobstructed path through the chip to allow quantitative study of BAFUS dosing.

Low Frequency (~250 kHz): In an embodiment, compared with first-generation devices using higher frequency and power (e.g., ablation4), the tool's low-frequency transducer overcome the energy loss due to skull attenuation14 and requires less sonication energy to produce stable cavitation.15

Semi-Permeable Dual-Channel Crossflow: In an embodiment, the chip has two flow channels to represent the extracellular matrix (bottom) and luminal side (top) of the BBB. This configuration permits microbubbles to flow on one side of the chip, imitating the intravenous infusion of these bubbles in human and animal subjects.

As mentioned above current BBB organoid technology is incompatible with microbubble flow, and existing organ-on-chips do not have an ultrasound-transparent window. In an embodiment, the BBB-on-chip is the cornerstone of the research tool; comprising four frames separated by top and bottom membranes and a middle porous membrane, which forms a transparent ultrasound window through its center, and allows for quantitative delivery of FUS energy to the endothelial monolayer (FIGS. 8A-8B, 9, 32). In contrast to other BBB chip models, our product brings this technology into the benchtop, in-vitro model to de-risk candidate pharmaceutical assets by answering drug permeability questions before continuing to preclinical and human trials. Given these advantages, our tool enables further quantitative studies of the BAFUS BBBO process for drug applications.

Overall, our BAFUS tool using BBB-on-Chips informs trial investigators early in the development process in more efficient use of resources and participants' time.

BBB-chip device and US-transparency: In an embodiment, our chip design (FIGS. 8A-8B, 9, 32); comprises four 1 mm-thick Polydimethylsiloxane (PDMS) frames modified through soft lithography to create the geometry for semi-permeable dual channel crossflow. The frames are stacked vertically with a 0.4 um porous, 11 um-thick polyethylene terephthalate (PET) membrane to separate the two channels and 20 um-thick PDMS membranes on the top and bottom of each channel frame to contain their respective volumes. Four 1 mm diameter holes are scored on the top-most frame and membrane, while the top channel frame and the PET membrane will score holes to allow for flow in the bottom channel. A 3D-printed adaptor allows external tubing connections for fluid flow. A needle hydrophone characterized the transparency of the device window; Negative peak pressure (NPpeak) at the focus was 100% and 98.7% of the original value for normal and 31° beam incident angles, respectively.

In an embodiment, the FUS system comprises a waveform generator, an amplifier, a 1 MHz US transducer, a cavitation monitoring transducer, and a digital oscilloscope with fast Fourier transform (FFT) capability (FIG. 26A). A 3D printed fixture holds the BBB-chip and the US transducers to ensure the focal beams of the transducers going through the center of the chip US window. The fixture is immersed in a water bath with US absorbing pads during BAFUS. The BAFUS treatment consisted of a burst signal of 10 ms at 1 MHz with a Vrms of 100 mV repeated at 1 Hz.

In an embodiment, Nanobubble (NB) is fabricated according to literature13 using a mixture of lipids DBPC:DPPA:DPPE:mPEG-DSPE equal to 6:1:2:1 and C3F8 gas. Clear sub/super-harmonic peaks were seen during BAFUS treatment when NBs were inside the BBB-chip (FIG. 27D). The subharmonic peak disappears ~20 sec of BAFUS treatment for static NB solution in the channel but can be maintained for over 5 mins for a constant flow. It was used to identify the optimal NB dilution and flow rate to be 100× and 250 ul/min, respectively.

BBB-chip by Caco-2 and BAFUS BBBO: In an embodiment, Caco-2 cells, which can generate tight junction barriers, are formed into a monolayer and used as a BBB surrogate.16 An optimized cell seeding density of 450 k/cm2 with 24-hr culture was used to generate a barrier with TEER 400-500 ohm-cm2 measured in a transwell insert with the same PET membrane as in the BBB-chip.17 Then, the same barrier is formed in the BBB-chip, and the permeabilities of the Caco-2 BBB-chip barrier to two tracers (Lucifer Yellow and 70 kD-Dextran labeled with TMR dye) are measured at different conditions. FIG. 25 shows the results of BBBO for a BAFUS treatment of 120 sec. The porous PET membrane without cells served as the control. The Caco-2 barrier before BAFUS served as the baseline permeability, which was significantly lower than the control. The post-BAFUS time points of 0 and 24 hrs showed that the barrier was opened by BAFUS and partially recovered for the 70 kD-Dextrane tracer. There was no significant recovery for the Lucifer Yellow tracer.

iPSC differentiation to BMVEC and immunofluorescence for TJs: In an embodiment, human iPSCs differentiate into BMVEC using a hypoxia condition.18 First passage iPSC-derived BMVECs from two custom lines are grown on gelatin-coated transwells to 100% confluence, then fixed and stained with primary antibodies rabbit anti-TJP1 (HPA001636) or mouse anti-CD31 (MMS-484R), then incubated with appropriate secondary antibodies (FIG. 29).

Construct the 250 kHz BAFUS BBB-chip tool and characterize MB stable cavitation parameters: In an embodiment, we integrate the clinically relevant US transducer and microbubble with a BAFUS setup and BBB-chip to establish a working platform for subsequent studies.

Sonication setup: In an embodiment, a FUS transducer with a center opening and a cavitation monitoring transducer (Y-117 and Y-107 from Sonic Concept, Bothell, WA) is used in concentric format. The FUS beam has a normal incident angle to the BBB-chip window. A fixture is 3D printed to hold the transducers and the chip together so that the transducers are focused on the cell membrane within the chip window. The fixture is submerged into a 37° C. water bath with degassed deionized water for BAFUS treatment. US-absorbing pads are placed at bottom and sidewalls of the bath to reduce scattered US energy. The BAFUS signal is generated by a waveform generator (Siglent SDG1032X) and amplified by an ENI 240L amplifier before outputted to the FUS transducer with maximum power of 40 W and maximum focal pressure of 1.5 MPa. The cavitation signal is collected by our digital oscilloscope (Siglent SDS1104X-E) and analyzed by FFT.

BBB-chip and US-transparency: In an embodiment, a design with a window size of ~8 mm is used to fabricate the BBB chip device to accommodate the increased beam diameter of 6 mm of the new transducer. The FUS beam diameter and focal pressure are measured by a 1 mm needle hydrophone (from Onda) mounted on a X-YZ stage. The transparency of the device window is verified by comparing the focal pressures with and without the device.

MB cavitation characterization: In an embodiment, cell-less BBB-chips are used to identify the ranges of BAFUS parameters for stable MB cavitation inside the chip, including focal pressure, burst condition, MB dilution factor and flow rate. MB use conditions are mimicked from our use successful. The MB is diluted in culture medium and flowed through the bottom channel of the device with top channel filled with medium without MB. The focal pressures of 0.25, 0.5, 0.75, 1 and 1.25 MPa are tested. The burst is 2.5 ms with 4 Hz repetition rate. 10×, 100×, 1000× dilution factor are tested. The flow rate is 0 (static), 1×, 2×, 3×, 4×, 5× channel volume/min (the chip channel volume is 110 uL). The strength of the harmonics peaks and the noise floor are used to identify the onset of inertial cavitation, which will cause cell damage. The strength of the peak is also used to identify the dilution factor and flow rate to maintain a stable cavitation signal over planned BAFUS treatment duration (e.g., 120 sec).

In an embodiment, conditions for onset of inertial cavitation and maintaining stable cavitation during BAFUS treatment are identified using the new BAFUS BBB-chip setup.

In an embodiment, the new BAFUS platform is established with the new transducers, BBB-chip and MBs. The chip window remains even more transparent for the lower US frequency (with a longer wavelength). The onset of focal pressure for inertial cavitation is ~1 MPa,19 and replenishment of MB by flow (previously 3× channel volume/min) is able to maintain stable cavitation during planned BAFUS treatment.

Demonstrate BBBO of Caco-2 BBB-chip in the new BAFUS system: BAFUS BBBO of Caco-2 BBB-chip further validates the new BAFUS platform for in vitro cellular barrier disruption.

Formation of Caco-2 barrier in transwell and BBB-chip: In an embodiment, the Caco-2 barriers are formed. The 24-well transwell insert (0.4 um clear PET membrane) is first coated with a thin collagen I coating using 50 ug/ml rat tail collagen I solution in acetic acid and incubating at 37° C. for 1 hr. After PBS wash, 150 k cells in 200 ul medium are seeded in the top compartment for a seeding density of 450 k/cm2, and 900 ul medium are added in the bottom compartment. The barrier forms after 24 hr culture in a CO₂ incubator at 37° C. The TEER of the barrier reads 400-500 ohm-cm². The barrier in the BBB-chip is formed using the same collagen coating, cell seeding density and culture time as for transwell. After seeding, tubing to syringe pump is unplugged and the ports are capped for culture inside incubator. Both channels are filled with culture medium, and the 2 mm medium thickness is equivalent to the medium thickness in a T75 culture flask. Cell barrier is formed in either top or bottom channel as needed.

TEER measurement in transwell: In an embodiment, TEER of Caco-2 barrier in transwell is measured using a EVOM2 meter and a STX2 electrode (WPI Inc.).20 To minimize inaccuracy from temperature drop,21 the measurement is done within one minute in a biosafety cabinet after bringing the well plate out of the incubator (~4-6 inserts/measurement). The plate is put back to incubator for 10 min before the next measurement. With the 0.33 cm² insert membrane area and 10 k ohm maximum resistance, maximum 30 k ohm-cm2 TEER can be measured, which is enough to measure the expected ~10 k ohm-cm2 TEER for iPSC-derived BMVEC barrier.22

Permeability measurement in transwell and BBB-chip: In an embodiment, for transwell, the top compartment is loaded with 200 ul of 100 uM Lucifer Yellow, or 1 uM of 150 kD Dextran-CF750 dye (Biotium) in PBS. The bottom compartment is loaded with 900 ul PBS. The plate is put inside the incubator for 15 min before 100 ul solution from the bottom compartment being collected for measurement. The concentration of the tracer is measured using a BMG CLARIOstar Plus plate reader in fluorescence intensity mode. The apparent permeability is calculated using Pₐₚₚ=(C_{bot}·V_{bot})/(C₀·A·Δt),23 where C0 is the top compartment tracer concentration, Cbot and Vbot are the tracer concentration and volume of bottom chamber, A is the membrance area and Δt is the duration of permeation. Limit of detections (LODs) for the tracers are identified (~55 pM for LY and estimated 1 pM for 150 k-Dextrane-CF750) and C0 is ~107× higher than the LODs so that Papp down to 10-9 cm/sec can be measured. For BBB-chip, the channel with cells are loaded with tracers in PBS (C0) and the other channel by PBS using a dual channel syringe pump. The tubing is unplugged, and channel ports are capped for incubation inside the incubator for 15 min before the PBS volume is collected for permeability measurement. The flow rate during operation is tested to avoid any error due to pressure-driven flow.

BAFUS treatment and BBBO characterization: In an embodiment, the device is taken out of the incubator to connect a syringe containing MB in culture medium to the channel with cells. Then the device is put into the fixture with the BAFUS transducers and submerged into the 37° C. water bath. The MB solution flows with the pretested dilution factors and flow rates (Aim 1A) to fill the channel first, and BAFUS treatment starts. Table 6 lists the expected BAFUS conditions: US burst: 250 kHz US with 2.5 ms at 4 Hz; PNP (mPa): 0 (as control), 0.25, 0.5, 0.75, 1, 1.25; MB dilution and flow rate: ~100× and 3 chamber volume/min. The permeability is measured immediately before and after the BAFUS treatment to show any opening of the barrier. Three devices are used for each condition and student-t test is used to compare the permeabilities. For the control (i.e., PNP 0 MPa), there is no significant change of Papp from the MB solution and handling of the device. Significant increase (P<0.05) of Papp with active FUS energy indicates a barrier opening.

TABLE 6
Ultrasound Dose Configurations

| | 0 | 1 | 2 | 3 | 4 | 5 |
|---|---|---|---|---|---|---|
| PNP (kPa) | 0 | 250 | 500 | 750 | 1000 | 1250 |
| Frequency (kHz) | 250 | 250 | 250 | 250 | 250 | 250 |
| Microbubble | ~100x | | | | | |
| Concentration | | | | | | |
| Microbubble | 3x chamber volume/min | | | | | |
| Flow Rate | | | | | | |

A BAFUS treatment condition is identified to show significant Pₐₚₚ increase by the treatment.

In an embodiment, significant Pₐₚₚ increase by BAFUS is seen. The effect is more pronounced for the larger (150 kD-Dextran) tracer. The cell barrier is formed in both top and bottom channels to compare any effect due to MB buoyant. The current channel height of 1 mm is much larger than the size of capillary (~8 um). However, the barrier opening is caused by the rapid-cavitation shear generated by the bubbles near the cells,²⁴ not by the swelling of the bubbles (which reaches 3× of bubble size,²⁵ ~3-6 um, still smaller than the size of the capillary). Therefore, the large channel height does not prevent BAFUS BBBO.

Demonstrate iPSC-derived BMVEC barrier formation: Since the first human iPSC derived BMVEC a decade ago, the field has seen significant progresses with simplified and repeatable protocols and much wider availability.^{26,22} The TEER up to 10 k ohm-cm² has been reported, approaching the estimated value in-vivo. Some controversy exists regarding the exact phenotype of the cells.²⁷ However, it does not affect the testing of BAFUS opening of the BBB-like tight junction barriers indicated by the high TEER value.

iPSC cell culture: In an embodiment, human IMR90-4 (WiCell Research Institute), a mostly used iPSC line, is used. IMR90-4 is purchased and cultured using the Feeder Independent Pluripotent Stem Cell Protocol from WiCell using mTeSR Plus, ReLeSR and mFreSR reagents from Stem Cell Technologies.²⁸ Briefly, cells are cultured in Matrigel coated 6-well plates using mTeSR Plus medium that has enhanced pH buffering. The medium is changed daily or every other day (2 ml in a 6-well plate) until passage (when colonies are too dense/large). ReLeSR is used to selectively detach undifferentiated cells from the substrate for clump passaging. Manual removal of differentiated cells can also be used if needed. The suspended cells are either passed in a 1:50 ratio, or cryo-preserved in mFreSR.

iPSC differentiation of BMVECs and barrier formation in transwell and BBB-chip: In an embodiment, we use a simple fully defined serum-free protocol that yielded BBB endothelium with repeatable TEER in the range of 2 k-8 k ohm-cm² across multiple iPSC lines, with appropriate marker expression and active transporters (e.g., P-glycoprotein).²⁶ Any differentiated cells are scraped off the plate and the iPSCs are harvested by Accutase into single cell suspension and seeded on a Matrigel coated 6 well plate in E8 medium containing 10 uM Y-27632 at a density of 15.8 k/cm². 24 hr after seeding, the medium are switched to E6 to induce differentiation. E6 is changed daily for 4 days. Next, the cells are given human endothelial serum-free medium supplemented with 50× diluted B27, 20 ng/ml bFGF and 10 um retinoic acid for 48 hrs. Then cells are harvested by Accutase into single cell suspension and subcultured onto the transwell PET membranes coated in a mixture of 400 ug/ml collagen IV and 100 ug/ml fibronectin (minimum 4 hrs) at a ratio of ~2.9:1 (by area). Medium is then changed to basal endothelial medium with B27 but not bFGF and RA. TEER is measured daily, and the barrier with high TEER forms 24 hrs after the removal of bFGF and RA. Once the barrier with desired high TEER is achieved, the barrier formation process is transferred to the BBB-chip. The permeabilities of the tracers are measured in both transwell and BBB-chip. Comparable results indicate similar barriers in both transwell and BBB-chip.

High TEER (>1-2 k ohm-cm²) barriers are formed on transwell insert from human iPSC. In an embodiment, BMVEC barriers with TEER >1 k ohm-cm² are formed. Alternative iPSC lines or hypoxia culture¹⁸ can be used if needed.

Demonstrate barrier opening of iPSC-derived BMVEC (BBB)-on-chip by the BAFUS system. The iPSC-derived BMVEC (BBB)-chip will serve as an in vitro model closer to in vivo.

BAFUS treatment and BBBO characterization: In an embodiment, the iPSC BBB-chip is treated using the same BAFUS conditions listed in Aim 1B, and similar Pₐₚₚ measurement is used to identify any BBBO. Metric of Success: A BAFUS treatment condition is identified to show Pₐₚₚ increase by the treatment.

With tighter barrier formation than Caco-2 BBB-chips, we see lower Pₐₚₚ baseline and more BBBO opening even for the small LY tracer.

Establish BAFUS dose curves & onset of BBBO and BBB damage for a large and small tracer. Being able to establish the BAFUS dose curves for a drug and onsets of BBBO and BBB damage helps to identify the optimal BAFUS dose scheme to deliver drugs using BAFUS in a clinical setting.

Dose curves and onset of BBBO: In an embodiment, passive diffusion as measured by the apparent permeability Pₐₚₚ is a useful drug delivery mechanism. We define the dose curve as how Pₐₚₚ changes with BAFUS dose parameters for tracers LY and 150 kD-Dextran. The dose curve for 1) FUS PNP (0-1.25 MPa), 2) treatment time (~5-180 sec), 3) MB dilution and flow rate (as suggested by Aim1A) are generated. Other parameters, such as burst scheme, may be tested. This will establish the boundaries for onset of BBBO (i.e., Pₐₚₚ significantly larger than control).

Recovery and onset of damage: In an embodiment, it is also useful to access damage of BBB during BAFUS. One way is to use cell live/dead stain to check viability of the cells²⁹ after each BAFUS treatment. BAFUS BBBO usually recovers within 4-24 hrs.³⁰ So another way to assess damage is to measure barrier recovery after BAFUS treatment, i.e., temporal change of Pₐₚₚ immediately before and after, as well as 4 hrs and 24 hrs after BAFUS is measured and plotted. The onset of BBB damage is defined as inability for Pₐₚₚ to recover to the value immediately before BAFUS within 24 hrs. Repeatable dose and recovery curves can be established.

We expect the BAFUS BBBO dose curves and onsets of BBBO can be established. Pₐₚₚ increases with dose and reaches a plateau. BBB damage by live/dead staining increases when BAFUS transitions to inertial cavitation. Barrier recovery is visible. Supplements in culture medium, such as fibroblast growth factor, are tested to help full recovery.²⁹ This serves as the cornerstone to validate the model using animals and clinical trial data. In addition, we have identified optimal ultrasound dosages for 440 Da and 150 kDa tracers, outlining initial ultrasound doses we use in subsequent in-vivo validation studies towards a predictive BAFUS BBBO in vitro model.

### Example 4: Building an In Vitro Blood Brain Barrier Model to Test Disruption by Focused Ultrasound to Treat Glioblastoma

Treating glioblastomas and other cancers in the brain is particularly difficult due to the protective function of the blood brain barrier (BBB). Over 95% of drugs cannot reach cancerous tissues because of the tight junctions that form in brain capillaries. Recently, it has been shown that low intensity focused ultrasonic waves and infused microbubbles disrupt the BBB at targeted locations and may be an effective way to allow for the temporary passage of cancer-therapeutic drugs. However, the exact mechanism and potential genetic influence have not been elucidated. Here, we are developing an ultrasound transparent organ-on-chip model to test focused ultrasound with microbubble infusion treatment on an artificially produced BBB. Developing preclinical models of the BBB with inherent genomic differences in barriers will shed light on how individuals will respond to different ultrasound frequencies. In an embodiment, the in vitro BBB device is composed of two orthogonally stacked fluidic channels formed by a top and a bottom 20-μm-thick polydimethylsiloxance (PDMS) membranes (mixed in a 10:1 base to crosslinker ratio) and a middle 11-μm-thick polyester membrane with 3 μm pores. These membranes are fabricated by spinning PDMS on a silicon wafer at an optimized 3500 RPM. After several measures with different speeds, 3500 RPM successfully produced the desired 20 μm thickness for the membrane. The thin membrane channel walls are essential to deliver control ultrasound energy in the channels without hot spots caused by reflection and interference effects. An ultrasound system is constructed with a waveform generator, an amplifier, a 1 MHz ultrasound transducer. A 0.5 MHz hydrophone and a digital storage oscilloscope are used for cavitation monitoring. Nanobubbles (FUS Instruments) are used to assist the BBB disruption. To deliver the ultrasonic waves, the device is submerged in degassed DI water in a custom tank with both transducer and hydrophone focused on the center for the channels. Subharmonic signal is observed using the digital oscilloscope with Fast Fourier Transform (FFT). Additionally, endothelial cells are grown in adherent culture to validate tight junction formation through immunofluorescence.

### Blood-Brain Barrier Device and Ultrasound/Microbubbles Treatment System

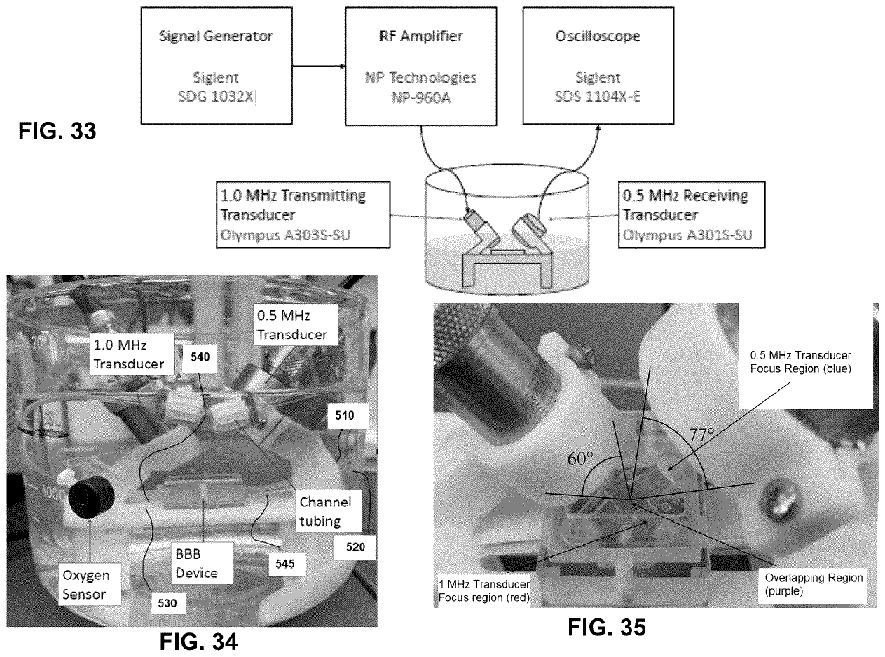

BBB Device Fabrication: In an embodiment, 4 PDMS frames re cured in an injection mold that are separated by a 20 μm PDMS membrane on top and bottom and a 11 μm porous polyester membrane in the middle. The US transparent membranes are created by spinning PDMS at 3500 RPM for one minute, and are then cured completely on the top and bottom channel frames. (FIGS. 33-35).

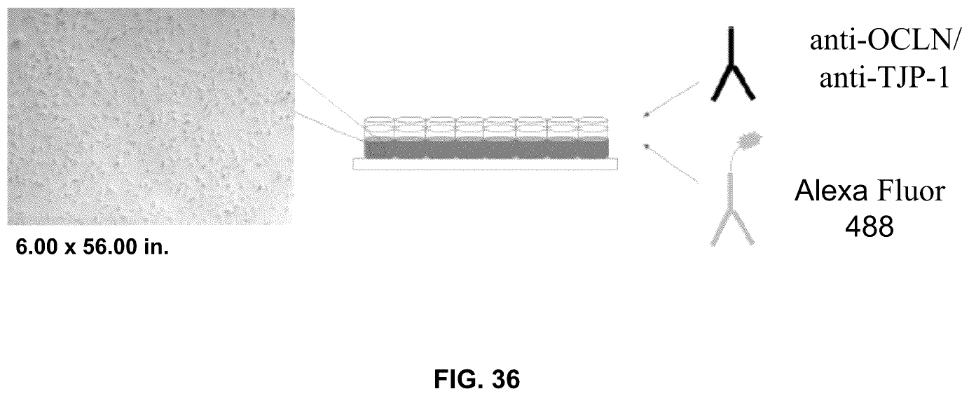

Tight Junctions: Commercially available endothelial cells (HBMEC-5i) cultured in collagen coated chamber slides. Immunofluorescence for key markers occluding, ZO-1 to validate tight junction formation and distribution. (FIG. 36).

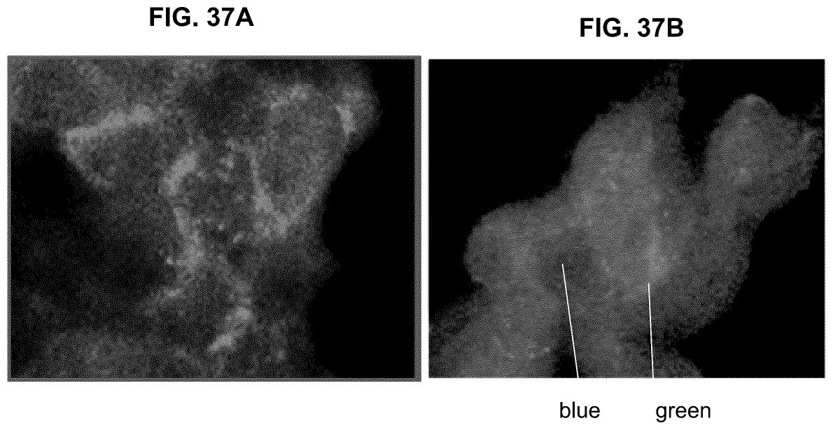

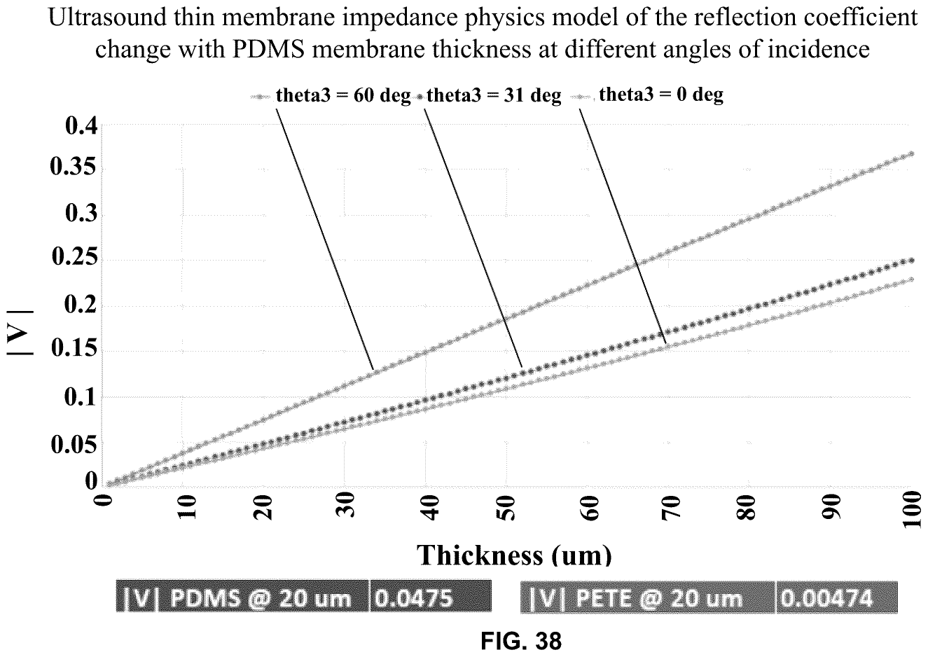

I. BBB Device Design (See FIGS. 8A-8C). II. Ultrasound/Microbubble Treatment (See FIGS. 37A-37C). III. Immunofluorescence (See FIGS. 38A-38B)

In an embodiment, the device sufficiently holds PBS without leaking thanks to the mechanical clamp holding down the needle barb frame to the PDMS frames. Furthermore, the 20 μm PDMS membrane proved to be relatively transparent to ultrasound as evident by the baseline frequency measured in FIGS. 37A-37C. The immediate increase in frequency across a broad range after applying 1 W of power to 10¹¹ bubbles/mL in the device suggest inertial cavitation. Furthermore, the 0.5 W power graph suggests that at lower powers, there may be a delay before the bubbles oscillate at their resonance frequency as the nanobubbles aggregate to form microbubbles.

Endothelial cells display high levels of occludin and ZO-1 to represent tight junction (TJ) formation. Junctional distributions are clear between endothelial cells and their neighboring cells. These constitute physiologically relevant barrier properties due to TJs being a significant BBB marker.

### References for Example 4

1. Beccaria K. et al. Journal of Neuro-Oncology 2020. doi.org/10.1007/s11060-020-03425-8
2. Chen K-T, Wei K-C and Liu H-L (2019) Theranostic Strategy of Focused Ultrasound Induced Blood-Brain Barrier Opening for CNS Disease Treatment. Front. Pharmacol. 10:86. doi: 10.3389/fphar.2019.00086
3. DeStefano, J. G., Jamieson, J. J., Linville, R. M. et al. Benchmarking in vitro tissue-engineered blood-brain barrier models. Fluids Barriers CNS 15, 32 (2018). doi.org/10.1186/s12987-018-0117-2

### Example 5: a Pulsed Low Intensity Focused Ultrasound Blood Brain Barrier Disruption Model for Glioblastoma Therapy

The blood-brain barrier (BBB) prevents 95% of drugs from reaching brain tumors [1].

In an embodiment, our BBB-On-Chip device is ultrasound transparent, facilitating bubble assisted focused ultrasound (BAFUS) medicated BBB disruption. The BBB-On-Chip is made of 4 polydimethylsiloxane (PDMS) frames each 1-mm-thick separated by top and bottom 20-μm-thick PDMS membranes and a middle 11-μm-thick 0.4-μm-porous polyester (PETE) membrane between two orthogonal channels. Peak-negative pressure was measured before and after the chip using a needle hydrophone and 1 MHz transducer in an anechoic-absorber-padded (AAP) water tank. The BAFUS delivery system comprises a 1 MHz transmitting and 0.5 MHz receiving transducer fixed in place at 60° to the device and submerged in a degassed, AAP water tank. A wave form generator and amplifier generate the signal and a digital storage oscilloscope monitors stable cavitation.

In an embodiment, nanobubbles are fabricated via lipid dissolution [2] and iPSC-derived endothelial cells are cultured with an optimized differentiation protocol [3].

In an embodiment, the BBB-On-Chip is observed to be ultrasound transparent with no change in detected signal after passing through the device.

In an embodiment, nanobubbles produced a subharmonic peak at 0.5 MHz, indicative of stable cavitation.

In an embodiment, key tight junction proteins can be visualized under confocal microscopy in iPSC-derived brain endothelial cells.

In an embodiment, by assessing multiple iPSC derived brain endothelial cell lines in the ultrasound transparent BBB-On-Chip, we can characterize safe parameters for BAFUS mediated BBB disruption and subsequent glioblastoma treatment in a genetically diverse population.

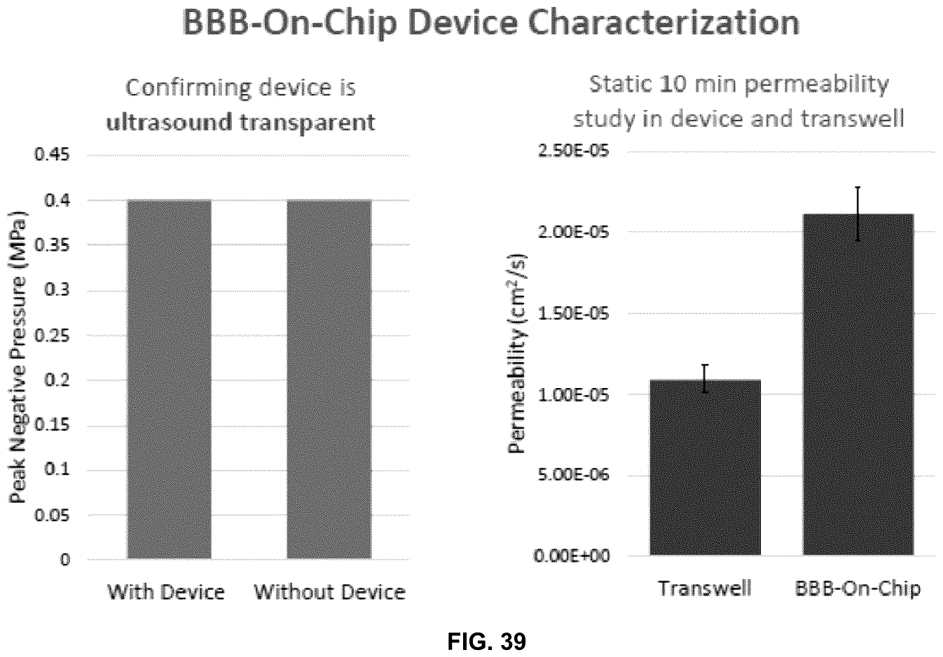

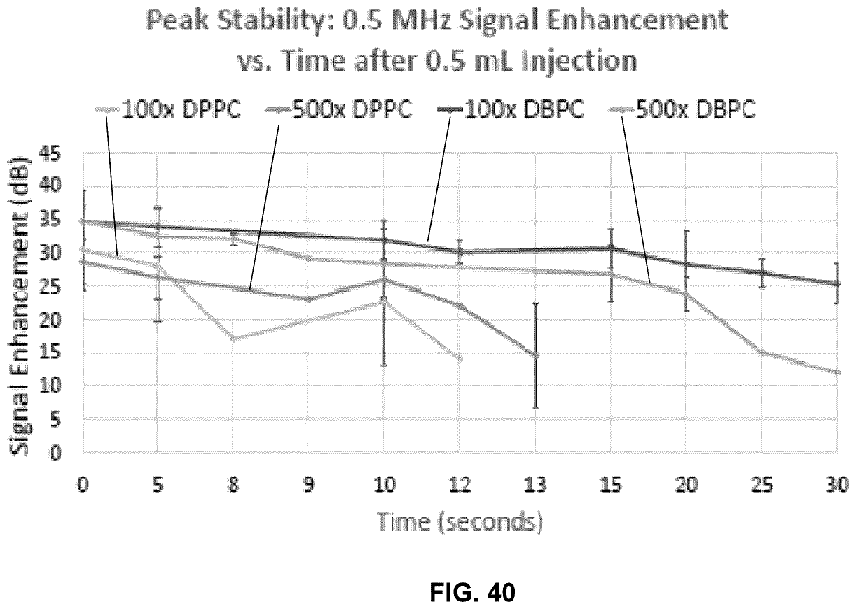

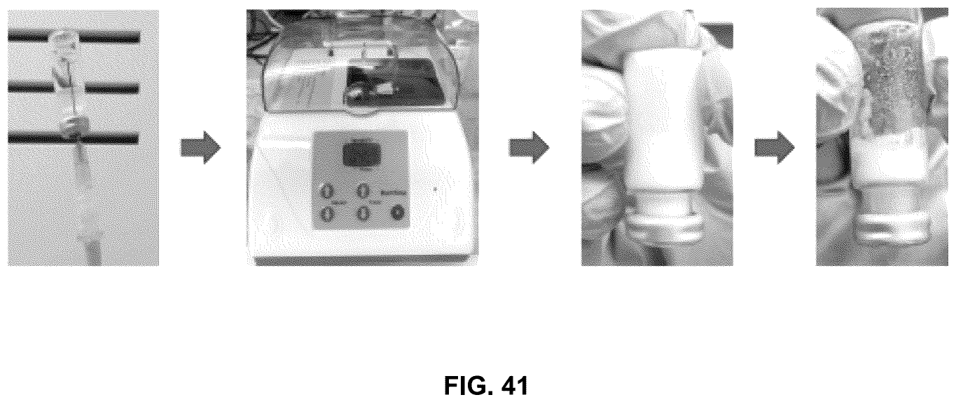

US-transparent BBB-On-Chip. See FIGS. 8A-8C. BAFUS setup to measure stable cavitation. See FIGS. 2, 26A. Hydrophone setup to measure ultrasound transparency. See FIGS. 26B-26C. Various results are summarized at, for example, FIGS. 15A-15B, 17A-17B, 18, 19, 28-29, 39-41.

The BBB-On-Chip is ultrasound transparent making it suitable to model BAFUS in vitro and permeably can be measured in the device to determine BBB tightness before and after BAFUS.

Nanobubbles have been successfully fabricated, detected, and observed under TEM. DBPC bubbles had the longest half life, greatest enhancement, and lasted over 5 minutes under flow, which is adequate for circulatory diffusion.

Coculture with astrocytes increased brain endothelial cell TEER. Brain endothelial cells have been successfully differentiated from iPSCs.

### References for Example 5

1. Abrahao, A. Nat Commun (2019) 10, 4373.
2. Cheng, B. A. Ultrasound in Medicine & Biology (2019) 45(8), 2174-2187
3. Park, T E. Nat Commun (2019) 10, 2621.

### Example 6: An Ultrasound-Transparent Organ-On-Chip Platform for Modeling Bubble-Assisted Focused Ultrasound (BAFUS) Cellular Barrier Disruption

Recent years have seen a significant growth in bubble-assisted focused ultrasound (BAFUS) to open cellular barriers (primarily in the brain) in a noninvasive and reversible way for drug delivery and liquid biopsy [1-3]. The procedure has been tested in animal models before the recent clinical trials. However, such tests were still empirical, and many questions remain, such as the optimal condition for barrier opening and the amount of drugs or types of biomarkers that can cross the cellular barrier. Animal models or clinical trials are expensive and low throughput. An in vitro model of the BAFUS barrier opening is highly needed that can help to optimize and predict the drug/biomarker barrier-crossing process.

It is traditionally challenging to model cellular interactions with ultrasounds (US) due to the large impedance mismatch between the aqueous environment of the cells and the plastic culture plates. Unintended “hot spots” with energy uncertainty up to 700% can form [4]. To address this issue, we report an US-transparent organ-on-chip device to model the BAFUS cellular barrier disruption process.

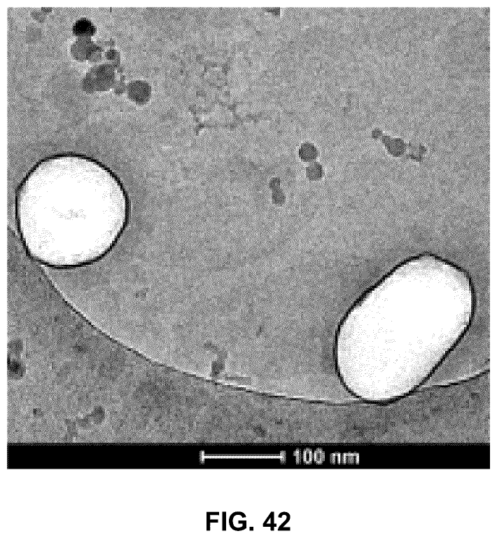

Ultrasound Setup. See FIGS. 2, 26A. Device Fabrication. See FIGS. 8A-8C, 9. Nanobubble Fabrication. Formulation-6 DBPC (DPPC):1 DPPA:2 DPPE:1 mPEG-DSPE in 5% glycerol and 0.6% pluronic L10→Replace Gas with C₃F₈→Activate through amalgamation→Centrifuge and dilute bubbles. See FIG. 42.

Device US-transparency Characterization: Theoretical calculation. See FIGS. 10, 11B (PETE (11 μm) T>99.99%), 11D (PDMS (20 μm) T>99.8%).

Experimental measurement is summarized in Table 7.

TABLE 7

| FUS Beam 31° to | | | |
|---|---|---|---|
| Device (#) | Pₚₑₐₖ (MPa) | Pₚₑₐₖ′ (MPa) | % Transmitted |
| 1 | 0.362 | 0.362 | 100 |
| 2 | 0.362 | 0.362 | 100 |
| 3 | 0.362 | 0.362 | 100 |
| 4 | 0.348 | 0.348 | 100 |

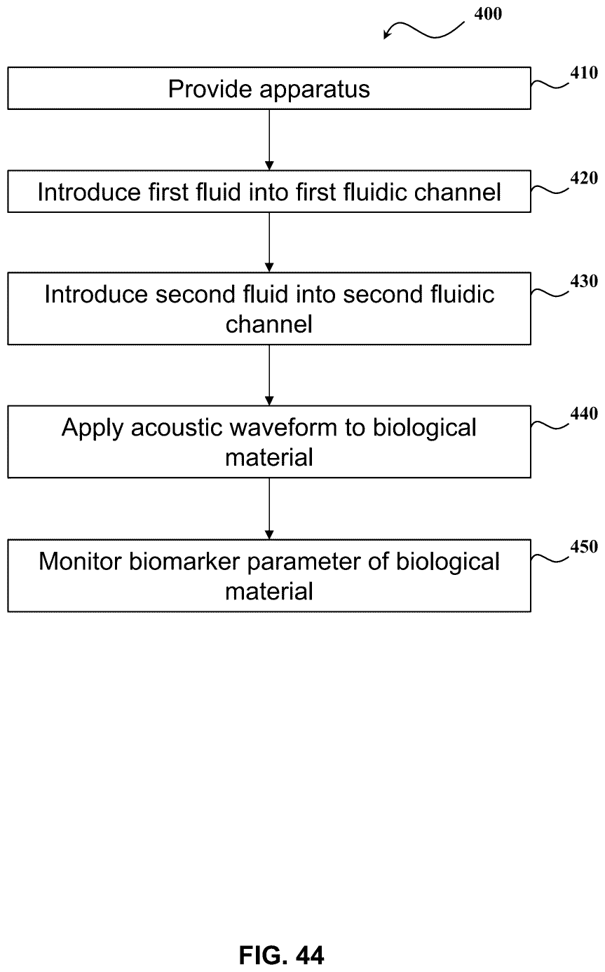

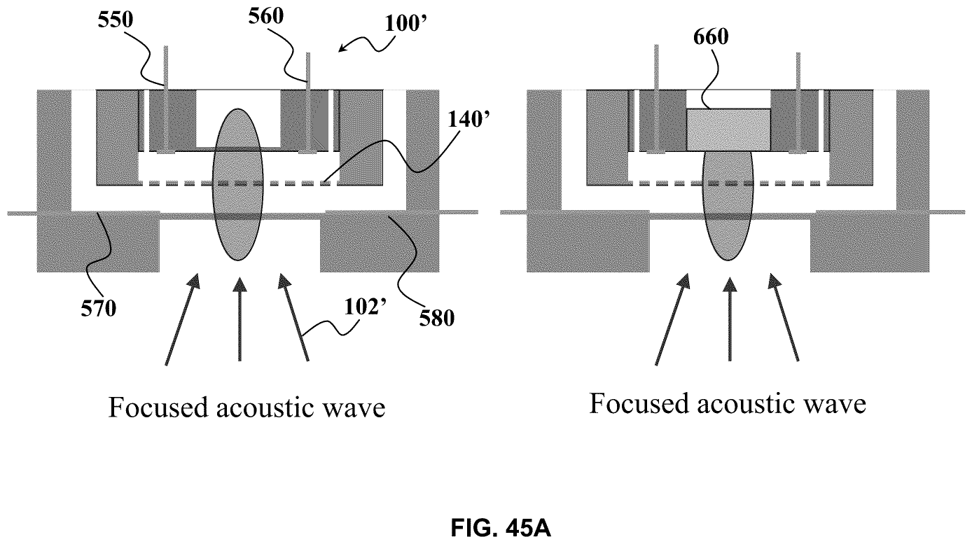

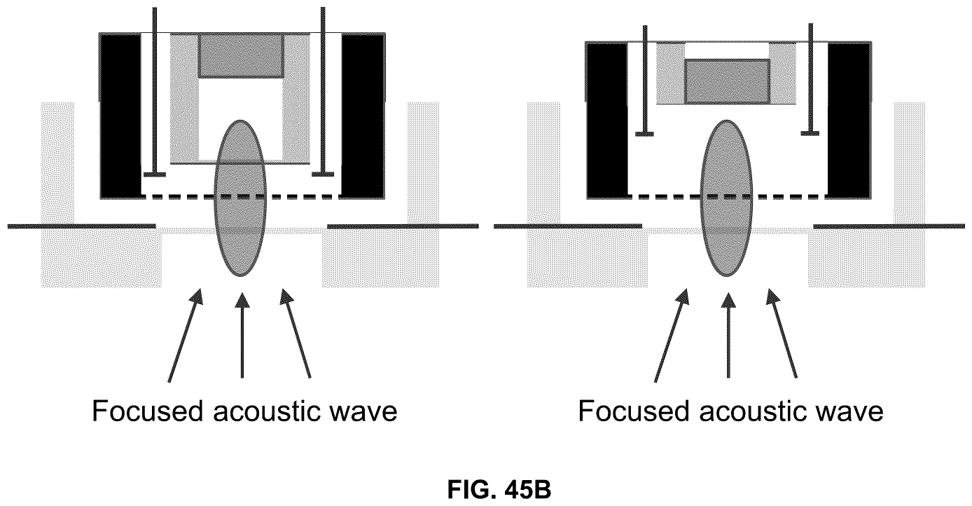

Nanobubble Characterization—See FIGS. 27C-27D, 44. Cellular Barrier Selection and Optimization-iPSC-derived Endothelial cells. See FIG. 21A. HBEC 5i Cell Line. See FIG. 21B. Caco-2 Cell Line. See FIG. 21C. TEER Measurement. See FIG. 20. Caco-2 cell line give the best cellular barrier property.

Caco-2 Barrier Optimization: A fast 24-hr Caco-2 barrier formation process was developed. See FIGS. 22-23. BAFUS Cellular Barrier Disruption.

Experimental Timeline: 1) t=−25 hr, Seed 150 k Caco-2 cells in Coated Device; 2) t=−1 hr, measure permeability (70 kD Dextran-TMR and LY); 3) t=0 hr, administer 2 min of 100 mVrms 1 MHz burst signal with 100×DBPC nanobubbles flowing at 15 ml/hr; 4) t=0 hr (+2 min), measure permeability (70 kD Dextran-TMR and LY); 5) t=24 hr, measure permeability after recovery (70 kD Dextran-TMR and LY).

Clear cellular barrier disruption and recovery were visible by 70 kD Dextran-TMR permeability measurement. See FIGS. 24A-24C, 25.

An US-transparent organ-on-chip BAFUS cellular barrier disruption platform has been developed. Preliminary cellular barrier BAFUS disruption has been demonstrated.

### References for Example 6

1. Meng, Y et al., Nat. Rev. Neur. 17, 8, 2021
2. Meng, Y et al., Neuro-Oncology 23(10), 1789, 2021
3. Brighi, C. et al., J of Controlled Release 345, 443-463, 2022
4. Leskinen, J. J. et al., Ultrasound in Med. & Biol. 38 (5), 777, 2012

### Example 7: Organ-On-Chip (OoC) Apparatus for Studying and Monitoring Acoustic Wave Interaction with Gas Bubbles and Disruption of Cellular Barriers

Provided are methods for studying and monitoring acoustic wave interaction with gas bubbles and disruption of cellular barriers. Thin films are used to form ultrasound-transparent windows to contain the OoC channels/compartments. A vessel/apical channel/compartment is used to flow gas bubble solutions. Hydrogel is used in the basolateral compartment for permeability imaging with spatial resolution. Cellular barriers are formed to separate the apical and basolateral compartments. A setup is used to perform bubble cavitation monitoring to correlate cellular barrier disruption in vitro with in vivo. In one embodiment, bubbles are mixed in human whole blood or equivalent for predictive cavitation doses. In another embodiment, the OoC apparatus can also contain vessel structures mimicking vascular network structures, surrounded by hydrogels compartment and contained by ultrasound transparent thin films. In a further embodiment, an additional perfusion channel/compartment adjacent to the hydrogel compartment can also be used, and all contained by the ultrasound transparent thin films. Applications include drug screening for ultrasound gas bubble disruption of blood-brain-barrier for brain drug delivery and also as a research tool.

Drug screening applications can use the ultrasound plus gas bubble to disrupt cellular barriers, including a hydrogel in a basolateral compartment, and cavitation monitoring to inform in vivo responses, and bubble mixed in blood or equivalent (i.e. solutions with blood cells or equivalent) for more predictive drug screening.

This example also provides additional apparatus embodiments, including to facilitate use of a hydrogel component, such as hydrogel containing vessel structures within an ultrasound transparent compartment. Preferably, the apparatus is configured to facilitate hydrogel filling, including hydrogel filling by a user.

This examples provides the ability to use precision dose human-derived in vitro models and physiologically based computation as New Approach Methodologies (NAMs) to understand, develop and optimize focused ultrasound (FUS) and gas bubble mediated blood-brain-barrier opening (BBBO) procedure for safe and effective brain drug delivery to treat different neurological disorders. To achieve this, we provide an ultrasound-transparent (UST) organ-on-chip (OoC) apparatus that allows consistent US dose delivery to cells in vitro.¹ We also differentiate brain microvascular endothelial cells (iBMECs) from human induced pluripotent stem cell (hiPSC) for in vitro BBB-on-chip modeling.

As a first application of our UST BBB-on-chip platform, we hypothesize that FUS BBBO to delivery drugs of different sizes in vitro using our UST BBB-on-chip is correlated with and can predict the FUS BBBO drug delivery in vivo through bubble cavitation monitoring. This apparatus can then be used as a preclinical drug screening tool for FUS BBBO, and is a useful alternative to animal models that FDA and NIH are currently promoting.²⁻⁷ The apparatus and methods provided herein are useful for commercialization and large-scale applications.

One goal is the development of predictive FUS BBBO in vitro model using the UST OoC device with hiPSC derived iBMECs, including to: 1) generate cavitation dose curves, identify acoustic dose thresholds for both subharmonic (SH) and broadband (BB) cavitations (AD_{SH} and AD_{BB}), both in vitro and in vivo, using a Definity-like gas bubble; 2) identify the acoustic doses of BBBO for different-size fluorescein-labeled dextrans (AD₃, AD₇₀, AD₅₀₀, AD₂₀₀₀ for 3, 70, 500 and 2,000 kDa dextrans) and tissue damage (AD_{TD}) in vitro using local tracer permeability imaging and cell dead-live assay; 3) establish AD₃, AD₇₀, AD₅₀₀, AD₂₀₀₀, and AD_{TD} in-vivo; identify in vitro-in vivo correlation.

Another goal is model validation using novel olive-derived nanovesicle (ODNV) delivery. ODNV is used as a model nanoparticle drug delivery carrier. The acoustic dose delivering ODNV in vitro (AD_{ODNV-in vitro}) and the in vitro-in vivo correlation identified as described above can be used to generate an acoustic dose for ODNV delivery in vivo (AD_{ODNV-in vivo}); successful delivery of ODNV delivery in vivo at this acoustic dose provides model validation.

Neurological conditions have recently been identified as the top contributor to the global disease burden⁸. In Arizona, 152,000 people are living with Alzheimer's;⁹ over 1.3 million adults have a mental health condition.¹⁰ Successful delivery of drugs to the brain is critical in treating numerous neurological conditions but is often hampered by the blood-brain barrier (BBB).

The BBB as a bottleneck for drug delivery to the brain: The BBB is part of the neurovascular unit (NVU) and is formed by a nonfenestrated endothelial cell monolayer along most vascular trees in the brain regulating low paracellular and transcellular permeability^{11,12}. While protecting the brain from neurotoxins and pathogens, the BBB also serves as a bottleneck for drug delivery^{13,14}, even in diseased states (e.g., acute ischemic stroke¹⁵, brain cancer¹⁶, and Alzheimer's Disease^{17,18}). Strategies including direct intracranial infusion, osmotic disruption, biomimetics, etc. have been investigated but are limited by lack of regional specificity, safety concerns, and the amount of drug/compound that can be delivered^{19,20}.

FUS BBBO as an emerging brain drug delivery method and the needs for predictive preclinical models: Recently, FUS with gas bubble has emerged as the only technology that can non-invasively deliver drugs to the brain at targeted locations by transiently opening the BBB^{21,22}. With its substantial benefits, FUS BBBO has seen a rapid translation into clinics: clinical trials has increased from 0 to 38 in the last 10 years²³. However, failed BBB opening and microhemorrhage were frequently reported^{17,24-26}, indicating the needs for better understanding and control of the process. As clinical applications of FUS BBBO continue to expand beyond the initial terminal brain diseases (glioblastoma, Alzheimer's etc.), there is a pressing need to fully understand its bioeffects.^{27,28} Novel modalities and systems without magnetic resonant (MR) guidance are also under development to make the technology more accessible, such as neuronavigation-guided FUS^{29,30}, US-guided FUS^{31,32}, and short pulsed theranostic US (ThUS).^{33,34} There are also needs to deliver novel nanoparticle drugs, such as liposomes, adeno-associated virus (AAV), or even stem cells for advanced drug, gene or cell therapies.³⁵⁻³⁸ All these developments can benefit significantly from predictive preclinical models.

Call for human-derived in vitro models (Challenge of FUS BBBO in vitro modeling; FDA modernization Act 2.0): Acoustic dose (pressure) is one of the most important factors in FUS BBBO³⁹. Due to standing wave formation, it is traditionally challenging to achieve accurate and consistent US acoustic dose on cells in vitro because cell culture-ware (e.g., flasks, well plates) materials have large acoustic impedance mismatches with the aqueous environment of cells⁴⁰. This can cause energy uncertainty as high as 700%⁴¹, which can lead to unreproducible results and erroneous conclusions on US dose effect⁴⁰.

There are ways to minimize standing wave effects⁴⁰, such as thin film US transparent window⁴², tightly focused US beam⁴³, tilted incident angle, ultrashort pulse⁴⁴, impedance matching hydrogel⁴⁵, placement of US absorbing materials⁴⁰ etc. However, their applications have been limited to bubble cavitation characterization⁴⁶, or US-cell interaction at non clinical-relevant conditions⁴⁴. There are limited reports on in vitro modeling of FUS BBBO^{18,47}, however, the acoustic fields are generally not well characterized, and they are usually limited to only comparing conditions with and without US and/or gas bubbles^{18,47}. Currently there is no in vitro model that allows continuous acoustic dose dependent study and optimization of FUS BBBO, and FUS BBBO has been developed almost exclusively in animals. However, significant differences could exist between human and animals, e.g., Carpentier et al. reported BBBO acoustic pressure much higher⁴⁸ and opening closure much faster (1 hr vs 6-24 hrs)⁴⁹ in humans than in animals, possibly due to physiological differences. With growing scientific recognition that animals do not provide adequate models of human health and disease⁵⁰, the U.S. Congress has passed the FDA Modernization Act 2.0 to allow alternatives to animal testing². In early 2025, FDA announced its plan to reduce animal testing and utilize NAMs (human-derived in vitro models, in silico and computational modeling etc.^{4,5}) for preclinical study. NIH also announced recently (on Jul. 7, 2025) that it no longer supports studies exclusively by animal models, but prioritizes human-focused NAMs.^{6,7}

OoC for in vitro modeling and Clinical Trial in a Dish: OoC, aka microphysiological system (MPS), is a microfluidic-based in vitro system that can recapitulate human organ-level physiology and pathophysiology with high fidelity⁵¹. Comparing with 3-dimenional (3D) organoid systems⁵², OoCs are more suitable for sensor integration and system characterization. To reduce the high attrition rate of traditional animal-based drug development process, Yildirim et al. has envisioned a Clinical Trial in a Dish concept for preclinical studies⁵³. In this concept, hiPSCs will be differentiated into different cell types to construct engineered tissues that are integrated into an array of OoC systems. Then sophisticated in vitro pharmacodynamics (PD) and pharmacokinetics (PK) analysis will be conducted together with artificial intelligence (AI) and machine learning (ML) to predict absorption, distribution, metabolism, excretion and toxicity (ADMET) outcomes. We envision OoC also as a promising platform for in vitro modeling of FUS BBBO, however, OoC modeling of FUS BBBO has yet to be demonstrated.

Bubble cavitation for FUS BBBO in vitro-in vivo correlation for brain drug delivery: For OoC model to be predictive in brain drug delivery, an in vitro-in vivo correlation needs to be established. This could be challenging due to the in vitro and in vivo differences. FUS BBBO in vivo is a highly complex process. It involves many parameters such as bubble pharmacokinetics⁵⁴⁻⁵⁶, nonuniform acoustic field, bubble cavitation in blood vessels with different lumen diameters^{46,57,58} and orientations to the FUS beam, polydispersity of gas bubbles, blood cell and perfusion⁵⁹ effects, vascular network structure⁶⁰⁻⁶³, FUS BBBO mechanisms and bioeffects^{21,22,28,47}. To fully understand the process, many aspects mentioned above are under active research. It is plausible to predict the BBBO and even drug concentration in brain parenchymal using physiologically based computation and the human-based OoC platforms described herein can play a significant role in this process. As a first application of our UST BBB-chip platform, we predict that a simple OoC modeling in vitro can be correlated with and be predictive of FUS BBBO in vivo in delivering drugs of different sizes through bubble cavitation monitoring. Accordingly, any of the apparatus provided herein can be utilized in a drug screening process to ensure appropriate disruption of BBB for drug delivery and access to the brain through the BBB.

Bubble cavitation is the most fundamental process in FUS BBBO. Bubble cavitation under acoustic fields can usually be classified as stable cavitation or inertial cavitation⁶⁴. At low acoustic pressure, stable cavitation generates harmonics of the fundamental frequency f (2f, 3f, . . . ). As pressure increases, bubbles are distorted and sub/ultra-harmonics (SH/UH, 0.5f, 1.5f, 2.5f, . . . ) appear in stable cavitation^{65,66 21}. Further increase of acoustic pressure changes the cavitation from stable to inertial with bubble collapse and shockwave/microjet formation,⁶⁷ which causes broadband (BB) background increase^{66,68}. It is believed that the mechanical forces from bubble cavitations are the causes of increased BBB permeability, and there is a strong correlation between acoustic pressure and BBBO, i.e. higher acoustic pressure can deliver drugs of larger size across the BBB³⁹. On the other hand, inertial cavitation is associated with cell damage and brain hemorrhage.^{69,70} With this strong correlation between gas bubble cavitation and FUS BBBO, we reason that similar bubble cavitation generates similar BBBO, despite the fact that acoustic pressures to generate the same cavitation in vitro (UST BBB-chip) and in vivo can be different (e.g. higher SH acoustic pressure threshold has been reported in smaller vessels⁴⁶, possibly due to a vessel confinement effect). Based on this rationale, it is possible to correlate BBBO in vitro and in vivo through bubble cavitation monitoring and develop UST BBB-chip as a predictive preclinical model for FUS BBBO. The cavitation monitoring is usually done by collecting the bubble cavitation signal using a Passive Cavitation Detector (PCD) and converting the signal into frequency domain using Fast Fourier Transform (FFT), then calculating doses of different frequency bands, e.g. harmonics, sub/ultra-harmonics, broadband⁶⁴.

Innovations of the UST OoC platform provided herein includes: 1) The microfabricated UST OoC apparatus allows accurate and consistent US dose delivery to cells in vitro.¹ This allows continuous acoustic dose study of the FUS BBBO process. 2) Correlation of FUS BBBO in vitro with that in vivo in delivering drugs of different sizes through bubble cavitation. This correlation can establish the UST BBB-chip platform as a predictive NAM to screen drugs for FUS BBBO brain drug delivery. It will also serve as a foundation for further UST BBB-chip based FUS BBBO studies. 3) Demonstration of FUS BBBO delivery of ODNVs. It will demonstrate feasibility of FUS BBBO and application of our UST BBB-chip to deliver novel nanoparticles, like pEVs.

In an embodiment the US system apparatus (FIG. 2) comprises a waveform generator (Siglent: SDG 1032X), an RF amplifier (NP Technologies: NP961), 1 MHz FUS transducer (Olympus A303S), 0.5 MHz focused transducer (Olympus A301S) as a Passive Cavitation Detector (PCD), a digital oscilloscope (Siglent: SDS 1104X-E), and a 1-mm needle hydrophone (Precision Acoustics) to measure pressure of acoustic field.

UST OoC device: We use thin films as UST windows for OoC devices. In our 1^{st} generation device, we used multilayer PDMS (polydimethylsiloxane) bonding with spin coated PDMS thin film (<20 μm). We also fabricated a 2^{nd} generation (Gen2) device with TransEndothelial Electrical Resistance (TEER) monitoring capability. The device was made of machined polycarbonate (PC) parts bonded by double-sided tape⁷². The medical grade double-sided tape (Adhesives Research: Arcare 90106NB) was patterned by xurography using a Silhouette Cameo cutter. The UST window was formed by 12.5 μm perfluoroalkoxy film (CS Hyde Company: 23-1/2PFA-24). The porous membrane was 0.4 μm PET (Sterlitech: 1300019). Gold TEER electrodes were fabricated on the top/bottom parts of the device by shadow mask and ebeam evaporation. The finished device was disinfected by 99% 2-propanol before sterilization by X-ray irradiation. FIG. 46A shows an exploded view of the Gen2 apparatus device parts, a finished device with a flexible printed circuit board (f-PCB) TEER cable, and US transparency characterization showing acoustic pressure loss <2%.

rt-TEER and temperature recording on chip: A computer with a custom Python™ software was used to collect real-time (rt) TEER and temperature data from an EVOM Manual meter (WPI) and a temperature sensor on the f-PCB. FIG. 46B shows that Caco-2-on-chip TEER and temperature recorded in real-time for 72 hrs. Caco-2 TEER dropped after 24 hrs due to the cytotoxicity of Cr adhesion layer of the gold electrodes. For this example, biocompatible Ti adhesion layer is used to avoid Cr cytotoxicity.

Bubble fabrication: We fabricate C3F8 core lipid-shell nanobubbles 73. We also fabricate clinical Definity-like microbubbles according to manufacturer's recipe74. The Definity-like bubble has been characterized by Dynamic Light Scattering (DLS) using a Zetasizer Ultra (Malvern Panalytical), showing a size ~1 μm, concentration ~1.5*1010/ml and zeta potential ~−2.07 mV, consistent with manufacturer's info and literature74,75.

Cavitation monitoring: Bubble echo is detected by the 0.5 MHz PCD and displayed by the digital oscilloscope in FFT frequency domain (data not shown). A camera is used to take pictures of the displays at different acoustic pressures. Subharmonic (SH) peak and broadband (BB) signal at 375 kHz are used to plot the SH and BB dose curves of the Definity-like bubble (FIG. 46C).

iBMEC differentiation for in vitro BBB modeling: Currently, human primary BMECs still cannot form a barrier with physiological TEER for drug permeability evaluation76. We followed a simplified fully-defined protocol to differentiate the iBMECs77, and achieved a BBB barrier with TEER over 5,500 ohm-cm2, in the range of the estimated in vivo values of 1500-8000 ohm-cm2 76,78 (FIG. 46D).

FUS Caco-2 barrier opening: FIG. 46E shows our preliminary results of FUS opening of a Caco-2-chip barrier for lucifer yellow (LY) and 70 kDa-dextran-TMR using global permeability measurement. The barrier is opened for both tracers, but recovered only for 70 kDa-dextran, but not for lucifer yellow in 24 hrs.

Oliver-derived nanovesicles (ODNVs): Plant-based extracellular vesicles (pEVs) are a novel class of EVs that shared similar structure with animal EVs but with lower toxicity, immunogenicity, inflammation, and even with therapeutic effects79-82. They are also suitable for manufacturing due to their availability. We have recently extracted and characterized nanovesicles from olive (ODNVs) 79, a plant often used in healthy diets. We are also extracting other novel pEVs that can be cultivated by vertical farming.

Experimental Methodology: FUS System: We use our existing FUS system. Its 1 MHz frequency has been used both in clinical trials (SonoCloud)16,48,49 and in small animal experiments83,84. Animals: Healthy adult female C57BL/6J mice (6-8 weeks, The Jackson Laboratory) will be used as the in vivo model 39,85. Mouse skull has low insertion loss, aberration and phase shift at 1 MHz86, and will allow us to perform transcranial FUS BBBO to avoid the complication of craniotomy.

UST BBB-chip local permeability imaging and optimization: Because FUS is used, the in vitro BBB will be opened only around the beam focal point. To image the BBBO with spatial resolution, the bottom compartment of the device will be filled with hydrogel so that dextran tracers across the BBB can be fixed after opening and imaged by confocal fluorescent microscopy. Sterile solution of 10% (w/v) porcine gelatin combined with microbial transglutaminase will be used as the hydrogel due to its compatibility with iBMEC culture87. COMSOL will be used to simulate the acoustic field inside the device with the hydrogel layer. Our Gen1 18-μm-thick PDMS film device showed standing wave variation of +/−5% inside the device by COMSOL. Thinner films, such as 2.5 μm PET (Chemplex Industries, Inc.), will be tested to further reduce the pressure variation. The flatness of the center porous PET membrane sometimes was an issue for cell imaging. We will test a setup to stretch the membrane to obtain flat porous PET membrane in the device.

Bubble fabrication, characterization and optimization: Definity-like microbubble will be fabricated using a chloroform-free process74,88. More specifically, lipids (DPPA, DPPC and DPPE-mPEG5000) will be dissolved in propylene glycol, heat for 1 h at 750 C, hydrated by PBS/Glycerol, then sonicate for 10 min before being aliquoted into 3 ml vials and stored at 40 C. Before use, air will be exchanged with C3F8 gas, and the vial will be activated by VialMix. The bubbles will be characterized by DLS. Even though our initial results were promising, we did observe some batch-to-batch variation. We plan to reduce the variation through reagent handling and storage condition optimization.

Bubble cavitation dose curves and ADSH and ADBB (in vitro and in vivo): Bubble gas volume equivalent to 7.5 μl/kg will be used initially and adjusted if needed. This gas volume dose is within the range used for small animals89 and is ~5 times higher than the clinical US contrast imaging dose. For in vitro, existing setup that focuses both transducers to the center of the OoC BBB barrier will be used. 96 nl/ml bubble diluted in live cell imaging solution (assuming a blood volume of 78 ml/kg for female mice) will be flown through the top channel of the device (bottom chamber filled with hydrogel). The perfusion rate will be optimized to obtain consistent cavitation signal. Acoustic pressure sweep of 0.05 to 1 MPa at a step of 0.05 MPa will be performed. Instead of taking pictures of our oscilloscope display, the waveforms will be recorded, transferred to a computer, analyzed by MATLAB as described in literature90. We expect this to be more sensitive than using values at a single peak or specific frequency. Multiple cavitation dose curves, such as harmonic, subharmonic and BB dose curves will be plotted for subsequent correlation. Background without bubbles will be measured and subtracted. Since our PCD is at subharmonic, onsets of subharmonic (ADSH) and broadband (ADBB) will be identified. Additionally, we will study the blood cell effects (i.e. microbubble diluted in purchased human whole blood). Due to abundant red blood cells in blood, there is a cell-free layer (CFL, ~0.5-5 μm) in large vessels91-93. We expect this will allow our BBB-chip to mimic in vivo better.

For in vivo, a Kopf Precise small animal stereotaxic “U” frame will be used. A custom probe94 will be made to target the parietal bone of mouse skull (1 mm posterior and 2 mm lateral to bregma, a possible treatment site for ischemic stroke/reperfusion). Targeting accuracy will be characterized by a hydrogel phantom with BSA94. Mice will be anesthetized and head hair removed to show bregma and sagittal suture. A water tank with thin film bottom and US gel will be used to couple the transducers with the animal head94. Bubbles will be infused by tail vein to keep a constant cavitation signal during data collection. The cavitation signal should be similar to the initial cavitation signal from a 7.5 μl/ml bolus injection. Then cavitation data will be collected and analyzed as that in vitro. 6-10 mice will be tested, depending on signal variation (n=6-10).

OoC BBB barrier culture: iBMECs will be differentiated as before77. The phenotype will be confirmed by immunostaining of BMEC markers, such as Occludin, Claudin-5, VE-Cadhesin, GLUT1, PECAM-177. We have formed BBB on transwell insert. We will test the process to form the BBB barrier inside the OoC device. Seeding density similar to that of transwell will be used. Barrier formation will be monitored by rt-TEER. A protocol will be developed according to literature to cryopreserve the differentiated iBMECs to minimize lead time and lower the cost95. A gravity feeding apparatus will be used for BBB-on-chip medium change.

FUS BBBO (pulsing parameters, tracers, in vitro, in vivo): A standard 1 ms pulse length, 1 Hz pulse repetition frequency, and 120 sec will be used throughout the project. Fluorescein-labeled fixable dextrans of 3, 70, 500, 2000 kDa (size ranges from 2.3 nm to 54.4 nm) will be used as BBBO tracers39. For in vitro, bubble solution will be perfused as in cavitation monitoring. FUS BBBO will be performed after barrier TEER reaching >1500 ohm-cm2 76,96, an industrial standard. Then tracers of 1.14 mg/ml in live cell imaging solution will be added to the top chamber and incubate for 20 min. This dose is equivalent to 2 mg/mouse39, assuming a mouse mass ~22.5 g. Then the top chamber will be washed, and the device will be fixed. BBBO will be determined by comparing the fluorescent signal in the gel with a control using a confocal microscope (e.g. 10% signal increase for BBBO). The size and intensity of opened area will be determined. The damage of the BBB will be characterized by Invitrogen LIVE/DEAD Viability/Cytotoxicity kit, and barrier TEER recovery time.

For in vivo, previously identified bubble condition will be used for tail vein infusion, and FUS BBBO will be performed in the right brain hemispheres, with the left as a control. 2 mg/mouse tracers will be injected by tail vein after BBBO. After 20 min, the mice will be transcardially perfused, fixed; the brain will be cut into slides in horizontal direction as described in literature39,97. BBBO area and intensity will be characterized by fluorescent confocal microscopy. H&E staining will be used to check microhemorrhage for tissue damage98. For each tracer and acoustic pressure, 4 mice will be used (n=4).

Generate cavitation dose curves, identify AD_{SH} and AD_{BB}. See above description.

Identify AD₃, AD₇₀, AD₅₀₀, AD₂₀₀₀ and AD_{TD} in vitro. Using AD_{SH} and AD_{BB} as reference points, FUS BBBO will be performed at 1) 50% AD_{SH}, 2) AD_{SH}, 3) AD_{BB} and 4) 1.5AD_{BB}. Additional acoustic dose points can be added as needed. The BBBO thresholds and AD_{TD} will be set as the lowest dose showing the bioeffects. Mice skull insertion loss (~18%^{99,100}) will be used to adjust the in vivo AD_{SH} and AD_{BB}.

Establish AD₃, AD₇₀, AD₅₀₀, AD₂₀₀₀, and AD_{TD} in-vivo; identify in vitro-in vivo correlation. AD₃ to AD₂₀₀₀ and AD_{TD} will be established as noted above, and their correlation to in vitro can be manifested by if they fall into the same regions as in vitro, e.g. <0.5 AD_{SH}, [0.5-1] AD_{SH}, [AD_{SH}, AD_{BB}] or [1-1.5] AD_{BB} etc.

Model validation using novel olive-derived nanovesicle (ODNV) delivery. ODNVs have a size ~100 nm⁷⁹. It falls outside of the studied tracer sizes (2.3 nm to 54.4 nm) and may not be the ideal nanoparticle to validate the model. However, it has the advantage of providing additional data points to improve the model. Smaller pEVs can be used if needed as they become available (e.g. our newly purified ~21 nm pEVs from Adriana Butter Head lettuce). AD_{ODNV-in vitro} will be identified first to predict the acoustic dose range for ODNV delivery in vivo. Successful delivery in vivo would validate our model and the correlation method. The pEVs will be labeled with PKH67 fluorescent membrane stain⁷⁹ for BBBO characterization.

### References for Example 7

1. Gu, J., Zenhausern, F. & Akkad, A. Apparatus for quantitative study of cellular interactions with mechanical waves. Application #PCT/US24/49098, Sep. 27, 2024 (pending).
2. Han, J. J. FDA Modernization Act 2.0 allows for alternatives to animal testing. Artif. Organs 47, 449-450 (2023).
3. Zushin, P.-J. H., Mukherjee, S. & Wu, J. C. FDA Modernization Act 2.0: transitioning beyond animal models with human cells, organoids, and AI/ML-based approaches. J. Clin. Invest. 133, (2023).
4. FDA. Roadmap to Reducing Animal Testing in Preclinical Safety Studies. Alternatives to Laboratory Animals https://www.fda.gov/media/186092/download?attachment (2025).
5. Commissioner, O. of the. FDA Announces Plan to Phase Out Animal Testing Requirement for Monoclonal Antibodies and Other Drugs. FDA https://www.fda.gov/news-events/press-announcements/fda-announces-plan-phase-out-animal-testing-requirement-monoclonal-antibodies-and-other-drugs (2025).
6. NIH to prioritize human-based research technologies | National Institutes of Health (NIH). https://www.nih.gov/news-events/news-releases/nih-prioritize-human-based-research-technologies.
7. Buntz, B. NIH announces end to funding for animal-only studies. Drug Discovery and Development https://www.drugdiscoverytrends.com/nih-announces-end-to-funding-for-animal-only-studies/(2025).
8. Steinmetz, J. D. et al. Global, regional, and national burden of disorders affecting the nervous system, 1990-2021: a systematic analysis for the Global Burden of Disease Study 2021. Lancet Neurol. 23, 344-381 (2024).
9. Public Health Action in Arizona|Alzheimer's Association. Alzheimer's Association https://www.alz.org/professionals/public-health/state-overview/arizona.
10. State Fact Sheets. National Alliance on Mental Illness (NAMI) https://www.nami.org/advocacy/state-fact-sheets/.
11. Daneman, R. & Prat, A. The Blood-Brain Barrier. Cold Spring Harb. Perspect. Biol. 7, a020412 (2015).
12. Sweeney, M. D., Zhao, Z., Montagne, A., Nelson, A. R. & Zlokovic, B. V. Blood-Brain Barrier: From Physiology to Disease and Back. Physiol. Rev. 99, 21-78 (2019).
13. Pardridge, W. M. The blood-brain barrier: Bottleneck in brain drug development. NeuroRX 2, 3-14 (2005).
14. Pardridge, W. M. Drug Transport across the Blood-Brain Barrier. J. Cereb. Blood Flow Metab. 32, 1959-1972 (2012).
15. Ronaldson, P. T. et al. CNS Drug Delivery in Stroke: Improving Therapeutic Translation From the Bench to the Bedside. Stroke 55, 190-202 (2024).
16. Carpentier, A. et al. Repeated blood-brain barrier opening with a nine-emitter implantable ultrasound device in combination with carboplatin in recurrent glioblastoma: a phase I/II clinical trial. Nat. Commun. 15, 1650 (2024).
17. Lipsman, N. et al. Blood-brain barrier opening in Alzheimer's disease using MR-guided focused ultrasound. Nat. Commun. 9, 2336 (2018).
18. Oikari, L. E. et al. Altered Brain Endothelial Cell Phenotype from a Familial Alzheimer Mutation and Its Potential Implications for Amyloid Clearance and Drug Delivery. Stem Cell

### Rep. 14, 924-939 (2020).

19. Dong, X. Current Strategies for Brain Drug Delivery. Theranostics 8, 1481-1493 (2018).
20. Wu, D. et al. The blood-brain barrier: Structure, regulation and drug delivery. Signal Transduct. Target. Ther. 8, 1-27 (2023).
21. López-Aguirre, M., Castillo-Ortiz, M., Viña-González, A., Blesa, J. & Pineda-Pardo, J. A. The road ahead to successful BBB opening and drug-delivery with focused ultrasound. J. Controlled Release 372, 901-913 (2024).
22. Perolina, E. et al. Translating ultrasound-mediated drug delivery technologies for CNS applications. Adv. Drug Deliv. Rev. 208, 115274 (2024).
23. Durham, P. G. et al. Current clinical investigations of focused ultrasound blood-brain barrier disruption: A review. Neurotherapeutics 21, e00352 (2024).
24. Gasca-Salas, C. et al. Blood-brain barrier opening with focused ultrasound in Parkinson's disease dementia. Nat. Commun. 12, 779 (2021).
25. Abrahao, A. et al. First-in-human trial of blood-brain barrier opening in amyotrophic lateral sclerosis using MR-guided focused ultrasound. Nat. Commun. 10, 4373 (2019).
26. Idbaih, A. et al. Safety and Feasibility of Repeated and Transient Blood-Brain Barrier Disruption by Pulsed Ultrasound in Patients with Recurrent Glioblastoma. Clin. Cancer Res. 25, 3793-3801 (2019).
27. Todd, N. et al. Secondary effects on brain physiology caused by focused ultrasound-mediated disruption of the blood-brain barrier. J. Controlled Release 324, 450-459 (2020).
28. Chen, S. et al. A review of bioeffects induced by focused ultrasound combined with microbubbles on the neurovascular unit. J. Cereb. Blood Flow Metab. 42, 3-26 (2022).
29. Chen, K.-T. et al. Neuronavigation-guided focused ultrasound for transcranial blood-brain barrier opening and immunostimulation in brain tumors. Sci. Adv. 7, eabd0772 (2021).
30. Pouliopoulos, A. N. et al. Safety evaluation of a clinical focused ultrasound system for neuronavigation guided blood-brain barrier opening in non-human primates. Sci. Rep. 11, 15043 (2021).
31. Deng, L., Yang, S. D., O'Reilly, M. A., Jones, R. M. & Hynynen, K. An Ultrasound-Guided Hemispherical Phased Array for Microbubble-Mediated Ultrasound Therapy. IEEE Trans. Biomed. Eng. 69, 1776-1787 (2022).
32. Singh, A. et al. Guiding and monitoring focused ultrasound mediated blood-brain barrier opening in rats using power Doppler imaging and passive acoustic mapping. Sci. Rep. 12, 14758 (2022).
33. Batts, A. J. et al. A multifunctional theranostic ultrasound platform for remote magnetogenetics and expanded blood-brain barrier opening. Brain Stimulat. 18, 1939-1951 (2025).
34. Batts, A. J. et al. Using a novel rapid alternating steering angles pulse sequence to evaluate the impact of theranostic ultrasound-mediated ultra-short pulse length on blood-brain barrier opening volume and closure, cavitation mapping, drug delivery feasibility, and safety. Theranostics 13, 1180-1197 (2023).
35. Morse, S. V., Mishra, A., Chan, T. G., T. M. de Rosales, R. & Choi, J. J. Liposome delivery to the brain with rapid short-pulses of focused ultrasound and microbubbles. J. Controlled Release 341, 605-615 (2022).
36. Tsitsos, F. N. et al. Characterization of microbubble cavitation in theranostic ultrasound-mediated blood-brain barrier opening for gene delivery. J. Controlled Release 385, 113986 (2025).
37. Lee, H. et al. Spatially targeted brain cancer immunotherapy with closed-loop controlled focused ultrasound and immune checkpoint blockade. Sci. Adv. 8, eadd2288 (2022).
38. Burgess, A. et al. Targeted Delivery of Neural Stem Cells to the Brain Using MRI-Guided Focused Ultrasound to Disrupt the Blood-Brain Barrier. PLOS ONE 6, e27877 (2011).
39. Chen, H. & Konofagou, E. E. The Size of Blood-Brain Barrier Opening Induced by Focused Ultrasound is Dictated by the Acoustic Pressure. J. Cereb. Blood Flow Metab. 34, 1197-1204 (2014).
40. Snehota, M., Vachutka, J., ter Haar, G., Dolezal, L. & Kolarova, H. Therapeutic ultrasound experiments in vitro: Review of factors influencing outcomes and reproducibility. Ultrasonics 107, 106167 (2020).
41. Leskinen, J. J. & Hynynen, K. Study of Factors Affecting the Magnitude and Nature of Ultrasound Exposure with In Vitro Set-Ups. Ultrasound Med. Biol. 38, 777-794 (2012).
42. Chan, L. Y. N. et al. Film-based Cell Culture Device and In Vitro Setup for Ultrasound Modulation. IEEE Trans. Ultrason. Ferroelectr. Freq. Control 1-1 (2025) doi:10.1109/TUFFC.2025.3569498.
43. O'Reilly, M. A., Huang, Y. & Hynynen, K. The impact of standing wave effects on transcranial focused ultrasound disruption of the blood-brain barrier in a rat model. Phys. Med. Biol. 55, 5251 (2010).
44. Helfield, B., Chen, X., Watkins, S. C. & Villanueva, F. S. Biophysical insight into mechanisms of sonoporation. Proc. Natl. Acad. Sci. 113, 9983-9988 (2016).
45. Mano, T., Grutman, T. & Ilovitsh, T. Versatile Ultrasound-Compatible Microfluidic Platform for In Vitro Microvasculature Flow Research and Imaging Optimization. ACS Omega 8, 47667-47677 (2023).
46. Katz, S. et al. Diameter-dependent assessment of microvascular leakage following ultrasound-mediated blood-brain barrier opening. iScience 26, 106965 (2023).
47. Conway, G. E., Paranjape, A. N., Chen, X. & Villanueva, F. S. Development of an In Vitro Model to Study Mechanisms of Ultrasound-Targeted Microbubble Cavitation—Mediated Blood—Brain Barrier Opening. Ultrasound Med. Biol. 50, 425-433 (2024).
48. Carpentier, A. et al. Clinical trial of blood-brain barrier disruption by pulsed ultrasound. Sci. Transl. Med. 8, 343re2-343re2 (2016).
49. Sonabend, A. M. et al. Repeated blood-brain barrier opening with an implantable ultrasound device for delivery of albumin-bound paclitaxel in patients with recurrent glioblastoma: a phase 1 trial. Lancet Oncol. 24, 509-522 (2023).
50. Statement on catalyzing the development of novel alternative methods. National Institutes of Health (NIH) https://www.nih.gov/about-nih/who-we-are/nih-director/statements/statement-catalyzing-development-novel-alternatives-methods (2024).
51. Ingber, D. E. Human organs-on-chips for disease modelling, drug development and personalized medicine. Nat. Rev. Genet. 23, 467-491 (2022).
52. Zhao, Z. et al. Organoids. Nat. Rev. Methods Primer 2, 1-21 (2022).
53. Yildirim, Z., Swanson, K., Wu, X., Zou, J. & Wu, J. Next-Gen Therapeutics: Pioneering Drug Discovery with iPSCs, Genomics, AI, and Clinical Trials in a Dish. Annu. Rev. Pharmacol. Toxicol. 65, 71-90 (2025).
54. Li, P. et al. Pharmacokinetics and pharmacodynamics of perfluoropropane after intravenous bolus injection of perflutren lipid microsphere injection (DEFINITY®) in healthy Chinese volunteers. BMC Pharmacol. Toxicol. 25, 6 (2024).
55. Navarro-Becerra, J. A., Song, K.-H., Martinez, P. & Borden, M. A. Microbubble Size and Dose Effects on Pharmacokinetics. ACS Biomater. Sci. Eng. 8, 1686-1695 (2022).
56. Prada, F. et al. Quantitative analysis of in-vivo microbubble distribution in the human brain. Sci. Rep. 11, 11797 (2021).
57. Gattegno, R. et al. Enhanced capillary delivery with nanobubble-mediated blood-brain barrier opening and advanced high resolution vascular segmentation. J. Controlled Release 369, 506-516 (2024).
58. Bezer, J. H. et al. Microbubble dynamics in brain microvessels. PLOS ONE 20, e0310425 (2025).
59. Raymond, S. B., Skoch, J., Hynynen, K. & Bacskai, B. J. Multiphoton Imaging of Ultrasound/Optison Mediated Cerebrovascular Effects in vivo. J. Cereb. Blood Flow Metab. 27, 393-403 (2007).
60. Løkkegaard, A., Nyengaard, J. R. & West, M. J. Stereological estimates of number and length of capillaries in subdivisions of the human hippocampal region. Hippocampus 11, 726-740 (2001).
61. Kubíková, T., Kochová, P., Tomášek, P., Witter, K. & Tonar, Z. Numerical and length densities of microvessels in the human brain: Correlation with preferential orientation of microvessels in the cerebral cortex, subcortical grey matter and white matter, pons and cerebellum. J. Chem. Neuroanat. 88, 22-32 (2018).
62. Smith, A. F. et al. Brain Capillary Networks Across Species: A few Simple Organizational Requirements Are Sufficient to Reproduce Both Structure and Function. Front. Physiol. 10, (2019).
63. Sargent, S. M. et al. Endothelial structure contributes to heterogeneity in brain capillary diameter. https://doi.org/10.1530/VB-23-0010 (2023) doi: 10.1530/VB-23-0010.
64. Mondou, P. et al. State of the art on microbubble cavitation monitoring and feedback control for blood-brain-barrier opening using focused ultrasound. Phys. Med. Biol. 68, 18TR03 (2023).
65. Lauterborn, W. Numerical investigation of nonlinear oscillations of gas bubbles in liquids. J. Acoust. Soc. Am. 59, 283-293 (1976).
66. Mondou, P. et al. State of the art on microbubble cavitation monitoring and feedback control for blood-brain-barrier opening using focused ultrasound. Phys. Med. Biol. 68, 18TR03 (2023).
67. Blake, J. R. & Gibson, D. C. Cavitation Bubbles Near Boundaries. Annu. Rev. Fluid Mech. 19, 99-123 (1987).
68. Yasui, K. Origin of the broad-band noise in acoustic cavitation. Ultrason. Sonochem. 93, 106276 (2023).
69. McDannold, N., Vykhodtseva, N. & Hynynen, K. Targeted disruption of the blood-brain barrier with focused ultrasound: association with cavitation activity. Phys. Med. Biol. 51, 793 (2006).
70. Xu, S. et al. Correlation Between Brain Tissue Damage and Inertial Cavitation Dose Quantified Using Passive Cavitation Imaging. Ultrasound Med. Biol. 45, 2758-2766 (2019).
71. Wasielewska, J. M. & White, A. R. “Focused Ultrasound-mediated Drug Delivery in Humans-a Path Towards Translation in Neurodegenerative Diseases”. Pharm. Res. 39, 427-439 (2022).
72. Smith, S., Sypabekova, M. & Kim, S. Double-Sided Tape in Microfluidics: A Cost-Effective Method in Device Fabrication. Biosensors 14, 249 (2024).
73. Cheng, B. et al. Influence of Nanobubble Concentration on Blood-Brain Barrier Opening Using Focused Ultrasound Under Real-Time Acoustic Feedback Control. Ultrasound Med. Biol. 45, 2174-2187 (2019).
74. DEFINITY® (Perflutren Lipid Microsphere) Diagnostic Ultrasound Enhancement. https://www.definityimaging.com/.
75. Ja'afar, F. et al. Surface Charge Measurement of SonoVue, Definity and Optison: A Comparison of Laser Doppler Electrophoresis and Micro-Electrophoresis. Ultrasound Med. Biol. 41, 2990-3000 (2015).
76. Shamul, J. G. et al. Meta-analysis of the make-up and properties of in vitro models of the healthy and diseased blood-brain barrier. Nat. Biomed. Eng. 9, 566-598 (2025).
77. Neal, E. H. et al. A Simplified, Fully Defined Differentiation Scheme for Producing Blood-Brain Barrier Endothelial Cells from Human iPSCs. Stem Cell Rep. 12, 1380-1388 (2019).
78. DeStefano, J. G., Jamieson, J. J., Linville, R. M. & Searson, P. C. Benchmarking in vitro tissue-engineered blood-brain barrier models. Fluids Barriers CNS 15, 32 (2018).
79. Zhao, Z. et al. Physical, biochemical, and biological characterization of olive-derived lipid nanovesicles for drug delivery applications. J. Nanobiotechnology 22, 720 (2024).
80. Fang, Z. & Liu, K. Plant-derived extracellular vesicles as oral drug delivery carriers. J. Controlled Release 350, 389-400 (2022).
81. Liu, M. et al. Therapeutic potential of plant-derived exosome-like nanoparticles for CNS diseases: advancements in preparation and characterization. Ann. Med. 57, 2537345 (2025).
82. Yu, Y. et al. Plant extracellular vesicles as emerging neuroprotective agents for central nervous system disorders. J. Adv. Res. https://doi.org/10.1016/j.jare.2025.03.042 (2025) doi:10.1016/j.jare.2025.03.042.
83. Géraudie, A. et al. Effects of Low-Intensity Pulsed Ultrasound-Induced Blood-Brain Barrier Opening in P301S Mice Modeling Alzheimer's Disease Tauopathies. Int. J. Mol. Sci. 24, 12411 (2023).
84. Chen, L. et al. Multimodal evaluation of blood-brain barrier opening in mice in response to low-intensity ultrasound and a claudin-5 binder. Nanotheranostics 8, 427-441 (2024).
85. Guo, Y. et al. Ultrasound frequency-controlled microbubble dynamics in brain vessels regulate the enrichment of inflammatory pathways in the blood-brain barrier. Nat. Commun. 15, 8021 (2024).
86. O'Reilly, M. A., Muller, A. & Hynynen, K. Ultrasound Insertion Loss of Rat Parietal Bone Appears to Be Proportional to Animal Mass at Submegahertz Frequencies. Ultrasound Med. Biol. 37, 1930-1937 (2011).
87. Faley, S. L. et al. iPSC-Derived Brain Endothelium Exhibits Stable, Long-Term Barrier Function in Perfused Hydrogel Scaffolds. Stem Cell Rep. 12, 474-487 (2019).
88. Abenojar, E. C. et al. Theoretical and Experimental Gas Volume Quantification of Micro- and Nanobubble Ultrasound Contrast Agents. Pharmaceutics 12, 208 (2020).
89. Song, K.-H. et al. Microbubble gas volume: A unifying dose parameter in blood-brain barrier opening by focused ultrasound. Theranostics 7, 144-152 (2017).
90. Martinez, P., Bottenus, N. & Borden, M. Cavitation Characterization of Size-Isolated Microbubbles in a Vessel Phantom Using Focused Ultrasound. Pharmaceutics 14, 1925 (2022).
91. Ong, P. K., Namgung, B., Johnson, P. C. & Kim, S. Effect of erythrocyte aggregation and flow rate on cell-free layer formation in arterioles. Am. J. Physiol.-Heart Circ. Physiol. 298, H1870-H1878 (2010).
92. Lauri, J., Bykov, A. & Fabritius, T. Quantification of cell-free layer thickness and cell distribution of blood by optical coherence tomography. J. Biomed. Opt. 21, 040501 (2016).
93. Salame, M. & Fenech, M. Key contributors to cell-free layer formation: An experimental investigation of hematocrit and shear rate gradient. Microvasc. Res. 162, 104859 (2025).
94. Bing, C. et al. Trans-cranial opening of the blood-brain barrier in targeted regions using astereotaxic brain atlas and focused ultrasound energy. J. Ther. Ultrasound 2, 13 (2014).
95. Wilson, H. K., Faubion, M. G., Hjortness, M. K., Palecek, S. P. & Shusta, E. V. Cryopreservation of Brain Endothelial Cells Derived from Human Induced Pluripotent Stem Cells Is Enhanced by Rho-Associated Coiled Coil-Containing Kinase Inhibition. Tissue Eng. Part C Methods 22, 1085-1094 (2016).
96. Mantle, J. L., Min, L. & Lee, K. H. Minimum Transendothelial Electrical Resistance Thresholds for the Study of Small and Large Molecule Drug Transport in a Human in Vitro Blood-Brain Barrier Model. Mol. Pharm. 13, 4191-4198 (2016).
97. Pandit, R. et al. Role for caveolin-mediated transcytosis in facilitating transport of large cargoes into the brain via ultrasound. J. Controlled Release 327, 667-675 (2020).
98. Tung, Y.-S. et al. In vivo transcranial cavitation threshold detection during ultrasound-induced blood-brain barrier opening in mice. Phys. Med. Biol. 55, 6141 (2010).
99. Choi, J. J., Pernot, M., Small, S. A. & Konofagou, E. E. Noninvasive, transcranial and localized opening of the blood-brain barrier using focused ultrasound in mice. Ultrasound Med. Biol. 33, 95-104 (2007).
100. Leinenga, G. et al. Scanning ultrasound-mediated memory and functional improvements do not require amyloid-β reduction. Mol. Psychiatry 29, 2408-2423 (2024).
101. Safe focused ultrasound-mediated blood-brain barrier opening is driven primarily by transient reorganization of tight junctions|bioRxiv. https://www.biorxiv.org/content/10.1101/2025.01.28.635258v1 (2025).
102. Conti, A. et al. Regulation of P-glycoprotein and Breast Cancer Resistance Protein Expression Induced by Focused Ultrasound-Mediated Blood-Brain Barrier Disruption: A Pilot Study. Int. J. Mol. Sci. 23, 15488 (2022).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Whenever a range is given in the specification, for example, a temperature range, a time range, a numerical range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

In general, the terms and phrases used herein have their art-recognized meaning, which can be found by reference to standard texts, journal references and contexts known to those skilled in the art.

1. An apparatus for manipulating a cellular interaction with a mechanical wave comprising:
a bottom layer forming a first fluidic channel;
a top layer forming a second fluidic channel;
an intermediate membrane positioned between the first fluidic channel and the second fluidic channel, wherein the intermediate membrane comprises a receiving surface, wherein the intermediate layer is in fluidic contact with the first fluidic channel and the second fluidic channel;
wherein the first and/or second fluidic channels and/or the receiving surface of the intermediate membrane is configured to support a biological material;
wherein the bottom layer, top layer and intermediate membrane are independently configured for mechanical wave transparency;
a mechanical wave generator configured to provide the mechanical wave to the biological material;
a biomarker parameter monitor configured to monitor a change in one or more biomarkers after provision of the mechanical wave.

2. The apparatus of claim 1, wherein the mechanical wave comprises an acoustic wave.

3. The apparatus of claim 1 configured for dynamic fluid flow, wherein:
the bottom layer comprises a first fluidic channel;
the top layer comprises:
a second fluidic inlet fluidically connected to the second fluidic channel and configured to introduce a second fluid to the second fluidic channel; and
a second fluidic outlet fluidically connected to the second fluidic channel and configured for removal of the second fluid from the second fluidic channel;
the biological material is positioned inside the first and/or second fluidic channels and/or on the receiving surface of the intermediate membrane;
a first fluidic inlet passing through the top layer and the intermediate membrane, fluidically connected to the first fluidic channel, and configured to introduce a first fluid to the first fluidic channel;
a first fluidic outlet passing through the top layer and the intermediate membrane, fluidically connected to the first fluidic channel and configured for removal of the first fluid from the first fluidic channel;
wherein the bottom layer, top layer and intermediate membrane are independently configured for acoustic transparency.

4. The apparatus of claim 1, wherein the top layer and bottom layer are configured to contain a first fluid in the first fluidic channel and a second fluid in the second fluidic channel, without any external introduction of fluid to the first fluidic channel or any external introduction of fluid to the second fluidic channel.

5. The apparatus of claim 1, wherein the biological material comprises:
an isolated biological tissue, cultured cells, and/or one or more monolayers of cells; or
a blood-brain interface or a model of a blood-brain interface.

6.-7. (canceled)

8. The apparatus of claim 1, wherein a portion of the second fluidic channel is positioned above a portion of the first fluidic channel to form a combined channel volume from an overlapping portion of the portion of the second fluidic channel with the portion of the first fluidic channel, wherein the first fluidic channel is orthogonal to the second fluidic channel.

9. (canceled)

10. The apparatus of claim 1:
the first fluidic channel has a non-uniform width with a maximum width positioned at a midpoint between the first fluidic inlet and the first fluidic outlet; and
the second fluidic channel has a non-uniform width with a maximum width positioned at a midpoint between the second fluidic inlet and the second fluidic outlet.

11.-13. (canceled)

14. The apparatus of claim 1, further comprising:
a first electrode and a second electrode each electrically connected to the first fluidic channel and a second electrode; and
a third electrode and a fourth electrode each electrically connected to the second fluidic channel;
wherein each of the first and second electrode are independently microfabricated on an inner surface of the first fluidic channel, and each of the third and fourth electrodes are independently microfabricated on an inner surface of the second fluidic channel.

15. (canceled)

16. The apparatus of claim 1, wherein the intermediate membrane is porous; comprises a material selected from the group consisting of polyester polytetrafluoroethylene (PETE), polydimethylsiloxane (PDMS), polycarbonate, and parylene; and has a thickness of between 0.01 μm and 20 μm and/or an average pore size of between 0.01 μm and 20 μm.

17.-18. (canceled)

19. The apparatus of claim 1, wherein:
the bottom layer comprises a bottom viewing frame positioned below the first fluidic channel, the bottom viewing frame comprising the first fluidic inlet and the first fluidic outlet; and
the top layer comprises a top viewing frame positioned above the second fluidic channel, the top viewing frame comprising the second fluidic inlet and the second fluidic outlet.

20.-21. (canceled)

22. The apparatus of claim 1, wherein:
the bottom layer further comprises a first thin layer positioned between the first fluidic channel and the bottom viewing frame, thereby preventing fluid from exiting the first fluidic channel other than at the first fluidic outlet and preventing fluid from entering the first fluidic channel other than at the first fluidic inlet; and
the top layer further comprises a second thin layer positioned between the second fluidic channel and the top viewing frame, thereby preventing fluid from exiting the second fluidic channel other than at the second fluidic outlet and preventing fluid from entering the second fluidic channel other than at the second fluidic inlet; wherein each of the thin layers comprises a material selected from the group consisting of polydimethylsiloxane (PDMS), parylene, poly-oxydiphenylene-pyromellitimide (Kapton), biaxially oriented polyethylene terephthalate (boPET; Mylar), and glass; and each of the thin layers have a thickness of between 0.01 μm and 100 μm.

23.-24. (canceled)

25. The apparatus of claim 1, wherein the mechanical wave generator is an acoustic wave generator configured to introduce an acoustic waveform to the biological material; wherein the acoustic wave generator is further configured to:
focus the acoustic waveform on the top layer; and
tilt from a position normal to the top layer to reduce acoustic waves reflected from the top layer back to the acoustic wave generator.

26. (canceled)

27. The apparatus of claim 25, the apparatus further comprising gas bubbles within a fluid, wherein the gas bubbles are configured to cavitate upon interaction with the acoustic waveform and thereby interact with the biological material.

28. The apparatus of claim 25, wherein the acoustic wave generator is configured to generate a waveform comprising: acoustic pulses with an acoustic frequency selected from the range of 100 kHz to 20 MHz, wherein each acoustic pulse has a pulse duration selected from the range of 100 ns to 2 s; a continuous acoustic wave with an acoustic frequency selected from the range of 100 kHz to 20 MHz; or an ultrasound waveform with the acoustic wave generator that is an ultrasound generator.

29.-30. (canceled)

31. The apparatus of claim 1, wherein the biomarker parameter provides a measure of: a cell morphology, mass transfer uptake, cellular barrier disruption, a protein, mRNA, and/or a polynucleotide; and/or cellular barrier disruption selected from the group consisting of: a permeability parameter of the biological material, a temperature parameter of the biological material, and a transendothelial electrical resistance (TEER) parameter of the biological material.

32. (canceled)

33. The apparatus of claim 1, further comprising:
a container configured to hold a fluid, wherein at least a portion of a surface(s) of said container is covered with an ultrasound absorbing material; wherein the container is filled with water, and wherein the bottom layer, the top layer, the intermediate membrane, and the biological material are submerged in said water; and the acoustic wave generator is submerged in said water.

34.-35. (canceled)

36. The apparatus of claim 33, wherein an air/water interface is covered with an ultrasound absorbing material.

37. (canceled)

38. The apparatus of claim 1, further comprising a hydrogel compartment positioned in the bottom layer.

39. The apparatus of claim 1, further comprising a transendothelial electrical resistance (TEER) monitor comprising a TEER cable electrically connected to the biological material.

40. A method of making an apparatus for the study of cellular interaction with ultrasound, the method comprising the steps of:
providing a bottom layer having a first fluidic channel;
providing a top layer having a second fluidic channel;
positioning an intermediate layer between the first fluidic channel and the second fluidic channel to form the first and second fluidic channels separated by the intermediate layer;
providing a biological material to the first fluidic channel and/or the second fluidic channel and/or the receiving surface of the intermediate membrane;
generating a mechanical waveform, wherein the top layer, bottom layer and intermediate membrane are configured to be mechanically transparent;
mechanically stimulating the biological material with the mechanical waveform; and
monitoring a change in one or more biomarkers after the mechanical stimulating step using a biomarker parameter monitor.

41.-49. (canceled)

50. A method of evaluating a cellular interaction with an acoustic wave, the method comprising:
providing the apparatus of claim 1;
introducing the first fluid into the first fluidic channel;
introducing the second fluid into the second fluidic channel;
wherein the introduced first fluid and second fluid are provided to the biological material;
applying an acoustic waveform to the biological material; and
monitoring a biomarker parameter of the biological material, thereby evaluating the cellular interaction with ultrasound.

51.-54. (canceled)
